SEQUENCE LISTING (197 sequences)

SEQ ID NO: 1 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 2 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 3 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 4 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 5 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKR

SEQ ID NO: 6 - protein, 108 aa, synthetic construct
DIQMTQSPSSVSASVGDRVTITCRASQGIRPFMAWYQQKPGKAPKLLIYAASSLQSGVPSRFSGSGSGTDFTLTISSLQPEDFATYYCQQANSFPITFGQGTRLEIKR

SEQ ID NO: 7 - protein, 108 aa, synthetic construct
DIQMTQSPSSVSASVGDRVTITCRASQGQRPFVSWYQQKPGKAPKLLIYAASSLQSGVPSRFSGSGSGTDFTLTISSLQPEDFATYYCQQANSFPITFGQGTRLEIKR

SEQ ID NO: 8 - protein, 108 aa, synthetic construct
DIQMTQSPSSVSASVGDRVTITCRASQGLRPFVNWYQQKPGKAPKLLIYAASSLQSGVPSRFSGSGSGTDFTLTISSLQPEDFATYYCQQANSFPITFGQGTRLEIKR

SEQ ID NO: 9 - protein, 108 aa, synthetic construct
DIQMTQSPSSVSASVGDRVTITCRASQGIRPFVDWYQQKPGKAPKLLIYAASSLQSGVPSRFSGSGSGTDFTLTISSLQPEDFATYYCQQANSFPITFGQGTRLEIKR

SEQ ID NO: 10 - protein, 108 aa, synthetic construct
DIQMTQSPSSVSASVGDRVTITCRASQGIRPFVAWYQQKPGKAPKLLIYAASSLQSGVPSRFSGSGSGTDFTLTISSLQPEDFATYYCQQANSFPITFGQGTRLEIKR

SEQ ID NO: 11 - protein, 108 aa, synthetic construct
DIQMTQSPSSVSASVGDRVTITCRASQGVRPFVNWYQQKPGKAPKLLIYAASSLQSGVPSRFSGSGSGTDFTLTISSLQPEDFATYYCQQANSFPITFGQGTRLEIKR

SEQ ID NO: 12 - protein, 108 aa, synthetic construct
DIQMTQSPSSVSASVGDRVTITCRASQGVRPFISWYQQKPGKAPKLLIYAASSLQSGVPSRFSGSGSGTDFTLTISSLQPEDFATYYCQQANSFPITFGQGTRLEIKR

SEQ ID NO: 13 - protein, 108 aa, synthetic construct
DIQMTQSPSSVSASVGDRVTITCRASQGIRPFIDWYQQKPGKAPKLLIYAASSLQSGVPSRFSGSGSGTDFTLTISSLQPEDFATYYCQQANSFPITFGQGTRLEIKR

SEQ ID NO: 14 - protein, 108 aa, synthetic construct
DIQMTQSPSSVSASVGDRVTITCRASQGVRPFVSWYQQKPGKAPKLLIYAASSLQSGVPSRFSGSGSGTDFTLTISSLQPEDFATYYCQQANSFPITFGQGTRLEIKR

SEQ ID NO: 15 - protein, 108 aa, synthetic construct
DIQMTQSPSSVSASVGDRVTITCRASQGIRPFVSWYQQKPGKAPKLLIYAASSLQSGVPSRFSGSGSGTDFTLTISSLQPEDFATYYCQQANSFPITFGQGTRLEIKR

SEQ ID NO: 16 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGKPVLDPITFGQGTRLEIKR

SEQ ID NO: 17 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGKPVFDPITFGQGTRLEIKR

SEQ ID NO: 18 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGIPVLGPITFGQGTRLEIKR

SEQ ID NO: 19 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGIPILGPITFGQGTRLEIKR

SEQ ID NO: 20 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 21 - protein, 117 aa, synthetic construct
EVQLLESGGGLVQPGGSLRLSCAASGFTFNHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 22 - protein, 117 aa, synthetic construct
EVQLLESGGGLVQPGGSLRLSCAASGFTFMHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 23 - protein, 117 aa, synthetic construct
EVQLLESGGGLVQPGGSLRLSCAASGFTFQWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 24 - protein, 117 aa, synthetic construct
EVQLLESGGGLVQPGGSLRLSCAASGFTFEWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 25 - protein, 117 aa, synthetic construct
EVQLLESGGGLVQPGGSLRLSCAASGFTFPWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 26 - protein, 117 aa, synthetic construct
EVQLLESGGGLVQPGGSLRLSCAASGFTFIHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 27 - protein, 117 aa, synthetic construct
EVQLLESGGGLVQPGGSLRLSCAASGFTFTHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 28 - protein, 117 aa, synthetic construct
EVQLLESGGGLVQPGGSLRLSCAASGFTFVHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 29 - protein, 117 aa, synthetic construct
EVQLLESGGGLVQPGGSLRLSCAASGFTFSHYHMLWVRQAPGKGLEWVSSISGAQWSTYYADSVKGRFTISRDNSKNTLYLQMNSLRAEDTAVYYCARFYDSFFDIWGQGTMVTVSS

SEQ ID NO: 30 - protein, 117 aa, synthetic construct
EVQLLESGGGLVQPGGSLRLSCAASGFTFSHYHMLWVRQAPGKGLEWVSSISGSVRSTYYADSVKGRFTISRDNSKNTLYLQMNSLRAEDTAVYYCARFYDSFFDIWGQGTMVTVSS

SEQ ID NO: 31 - protein, 117 aa, synthetic construct
EVQLLESGGGLVQPGGSLRLSCAASGFTFSHYHMLWVRQAPGKGLEWVSSINGSGWTTYYADSVKGRFTISRDNSKNTLYLQMNSLRAEDTAVYYCARFYDSFFDIWGQGTMVTVSS

SEQ ID NO: 32 - protein, 117 aa, synthetic construct
EVQLLESGGGLVQPGGSLRLSCAASGFTFSHYHMLWVRQAPGKGLEWVSSISGSHFPTYYADSVKGRFTISRDNSKNTLYLQMNSLRAEDTAVYYCARFYDSFFDIWGQGTMVTVSS

SEQ ID NO: 33 - protein, 117 aa, synthetic construct
EVQLLESGGGLVQPGGSLRLSCAASGFTFSHYHMLWVRQAPGKGLEWVSSINGFGRNTYYADSVKGRFTISRDNSKNTLYLQMNSLRAEDTAVYYCARFYDSFFDIWGQGTMVTVSS

SEQ ID NO: 34 - protein, 117 aa, synthetic construct
EVQLLESGGGLVQPGGSLRLSCAASGFTFSHYHMLWVRQAPGKGLEWVSSIGGDKRQTYYADSVKGRFTISRDNSKNTLYLQMNSLRAEDTAVYYCARFYDSFFDIWGQGTMVTVSS

SEQ ID NO: 35 - protein, 117 aa, synthetic construct
EVQLLESGGGLVQPGGSLRLSCAASGFTFSHYHMLWVRQAPGKGLEWVSSISGSMWSTYYADSVKGRFTISRDNSKNTLYLQMNSLRAEDTAVYYCARFYDSFFDIWGQGTMVTVSS

SEQ ID NO: 36 - protein, 117 aa, synthetic construct
EVQLLESGGGLVQPGGSLRLSCAASGFTFSHYHMLWVRQAPGKGLEWVSSINGMGATTYYADSVKGRFTISRDNSKNTLYLQMNSLRAEDTAVYYCARFYDSFFDIWGQGTMVTVSS

SEQ ID NO: 37 - protein, 117 aa, synthetic construct
EVQLLESGGGLVQPGGSLRLSCAASGFTFSHYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 38 - protein, 117 aa, synthetic construct
EVQLLESGGGLVQPGGSLRLSCAASGFTFSHYHMLWVRQAPGKGLEWVSSINGYGIQTYYADSVKGRFTISRDNSKNTLYLQMNSLRAEDTAVYYCARFYDSFFDIWGQGTMVTVSS

SEQ ID NO: 39 - protein, 117 aa, synthetic construct
EVQLLESGGGLVQPGGSLRLSCAASGFTFSHYHMLWVRQAPGKGLEWVSSINGKGWTTYYADSVKGRFTISRDNSKNTLYLQMNSLRAEDTAVYYCARFYDSFFDIWGQGTMVTVSS

SEQ ID NO: 40 - protein, 117 aa, synthetic construct
EVQLLESGGGLVQPGGSLRLSCAASGFTFSHYHMLWVRQAPGKGLEWVSSIGGDIRQTYYADSVKGRFTISRDNSKNTLYLQMNSLRAEDTAVYYCARFYDSFFDIWGQGTMVTVSS

SEQ ID NO: 41 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFDIWGQGTMVTVSS

SEQ ID NO: 42 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYDSHFDIWGQGTMVTVSS

SEQ ID NO: 43 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDNFDIWGQGTMVTVSS

SEQ ID NO: 44 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFREWGQGTMVTVSS

SEQ ID NO: 45 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFSPWGQGTMVTVSS

SEQ ID NO: 46 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFKPWGQGTMVTVSS

SEQ ID NO: 47 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFNPWGQGTMVTVSS

SEQ ID NO: 48 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFAPWGQGTMVTVSS

SEQ ID NO: 49 - protein, 117 aa, synthetic construct
EVQLLESGGGLVQPGGSLRLSCAASGFTFPWYRVHWVRQAPGKGLEWVSSIHTHRHITYYADSVKGRFTISRDNSKNTLYLQMNSLRAEDTAVYYCARFYDSFFDIWGQGTMVTVSS

SEQ ID NO: 50 - protein, 117 aa, synthetic construct
EVQLLESGGGLVQPGGSLRLSCAASGFTFPWYRVHWVRQAPGKGLEWVSSIHTHRNSTYYADSVKGRFTISRDNSKNTLYLQMNSLRAEDTAVYYCARFYDSFFDIWGQGTMVTVSS

SEQ ID NO: 51 - protein, 117 aa, synthetic construct
EVQLLESGGGLVQPGGSLRLSCAASGFTFPWYRVHWVRQAPGKGLEWVSSIHTGSQWTYYADSVKGRFTISRDNSKNTLYLQMNSLRAEDTAVYYCARFYDSFFDIWGQGTMVTVSS

SEQ ID NO: 52 - protein, 117 aa, synthetic construct
EVQLLESGGGLVQPGGSLRLSCAASGFTFPWYRVHWVRQAPGKGLEWVSSIHTHYQYTYYADSVKGRFTISRDNSKNTLYLQMNSLRAEDTAVYYCARFYDSFFDIWGQGTMVTVSS

SEQ ID NO: 53 - protein, 117 aa, synthetic construct
EVQLLESGGGLVQPGGSLRLSCAASGFTFPWYRVHWVRQAPGKGLEWVSSIHTQSKWTYYADSVKGRFTISRDNSKNTLYLQMNSLRAEDTAVYYCARFYDSFFDIWGQGTMVTVSS

SEQ ID NO: 54 - protein, 117 aa, synthetic construct
EVQLLESGGGLVQPGGSLRLSCAASGFTFPWYRVHWVRQAPGKGLEWVSSIRTDGTWTYYADSVKGRFTISRDNSKNTLYLQMNSLRAEDTAVYYCARFYDSFFDIWGQGTMVTVSS

SEQ ID NO: 55 - protein, 117 aa, synthetic construct
EVQLLESGGGLVQPGGSLRLSCAASGFTFPWYRVHWVRQAPGKGLEWVSSIQTHGVWTYYADSVKGRFTISRDNSKNTLYLQMNSLRAEDTAVYYCARFYDSFFDIWGQGTMVTVSS

SEQ ID NO: 56 - protein, 117 aa, synthetic construct
EVQLLESGGGLVQPGGSLRLSCAASGFTFPWYRVHWVRQAPGKGLEWVSSIHTHRNYTYYADSVKGRFTISRDNSKNTLYLQMNSLRAEDTAVYYCARFYDSFFDIWGQGTMVTVSS

SEQ ID NO: 57 - protein, 117 aa, synthetic construct; identical to SEQ ID NO: 56

SEQ ID NO: 58 - protein, 117 aa, synthetic construct
EVQLLESGGGLVQPGGSLRLSCAASGFTFPWYRVHWVRQAPGKGLEWVSSIKHGGRYTYYADSVKGRFTISRDNSKNTLYLQMNSLRAEDTAVYYCARFYDSFFDIWGQGTMVTVSS

SEQ ID NO: 59 - protein, 117 aa, synthetic construct
EVQLLESGGGLVQPGGSLRLSCAASGFTFPWYRVHWVRQAPGKGLEWVSSIHTHRPWTYYADSVKGRFTISRDNSKNTLYLQMNSLRAEDTAVYYCARFYDSFFDIWGQGTMVTVSS

SEQ ID NO: 60 - protein, 117 aa, synthetic construct
EVQLLESGGGLVQPGGSLRLSCAASGFTFPWYRVHWVRQAPGKGLEWVSSIHTHSDWTYYADSVKGRFTISRDNSKNTLYLQMNSLRAEDTAVYYCARFYDSFFDIWGQGTMVTVSS

SEQ ID NO: 61 - protein, 117 aa, synthetic construct
EVQLLESGGGLVQPGGSLRLSCAASGFTFPWYRVHWVRQAPGKGLEWVSSIHTHRQVTYYADSVKGRFTISRDNSKNTLYLQMNSLRAEDTAVYYCARFYDSFFDIWGQGTMVTVSS

SEQ ID NO: 62 - protein, 117 aa, synthetic construct
EVQLLESGGGLVQPGGSLRLSCAASGFTFPWYRVHWVRQAPGKGLEWVSSIRNENGWTYYADSVKGRFTISRDNSKNTLYLQMNSLRAEDTAVYYCARFYDSFFDIWGQGTMVTVSS

SEQ ID NO: 63 - protein, 117 aa, synthetic construct
EVQLLESGGGLVQPGGSLRLSCAASGFTFPWYRVHWVRQAPGKGLEWVSSIRSSGGFPYYHQKVKGRFTISRDNSKNTLYLQMNSLRAEDTAVYYCARFYDSFFDIWGQGTMVTVSS

SEQ ID NO: 64 - protein, 117 aa, synthetic construct
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSS

SEQ ID NO: 65 - protein, 117 aa, synthetic construct
EVQLLESGGGLVQPGGSLRLSCAASGFTFPWYRVHWVRQAPGKGLEWVSSIRSSGGFPYYNRRVKGRFTISRDNSKNTLYLQMNSLRAEDTAVYYCARFYDSFFDIWGQGTMVTVSS

SEQ ID NO: 66 - protein, 117 aa, synthetic construct
EVQLLESGGGLVQPGGSLRLSCAASGFTFPWYRVHWVRQAPGKGLEWVSSIRSSGGFPYYNHKVKGRFTISRDNSKNTLYLQMNSLRAEDTAVYYCARFYDSFFDIWGQGTMVTVSS

SEQ ID NO: 67 - protein, 117 aa, synthetic construct
EVQLLESGGGLVQPGGSLRLSCAASGFTFPWYRVHWVRQAPGKGLEWVSSIRSSGGFPYYHPSVKGRFTISRDNSKNTLYLQMNSLRAEDTAVYYCARFYDSFFDIWGQGTMVTVSS

SEQ ID NO: 68 - protein, 117 aa, synthetic construct
EVQLLESGGGLVQPGGSLRLSCAASGFTFPWYRVHWVRQAPGKGLEWVSSIRSSGGFPYYSLSVKGRFTISRDNSKNTLYLQMNSLRAEDTAVYYCARFYDSFFDIWGQGTMVTVSS

SEQ ID NO: 69 - protein, 117 aa, synthetic construct
EVQLLESGGGLVQPGGSLRLSCAASGFTFPWYRVHWVRQAPGKGLEWVSSIRSSGGFPHYNSVVKGRFTISRDNSKNTLYLQMNSLRAEDTAVYYCARFYDSFFDIWGQGTMVTVSS

SEQ ID NO: 70 - protein, 117 aa, synthetic construct
EVQLLESGGGLVQPGGSLRLSCAASGFTFPWYRVHWVRQAPGKGLEWVSSIRSSGGFPFYNSSVKGRFTISRDNSKNTLYLQMNSLRAEDTAVYYCARFYDSFFDIWGQGTMVTVSS

SEQ ID NO: 71 - protein, 117 aa, synthetic construct
EVQLLESGGGLVQPGGSLRLSCAASGFTFPWYRVHWVRQAPGKGLEWVSSIRSSGGFPYYNPFVKGRFTISRDNSKNTLYLQMNSLRAEDTAVYYCARFYDSFFDIWGQGTMVTVSS

SEQ ID NO: 72 - protein, 117 aa, synthetic construct
EVQLLESGGGLVQPGGSLRLSCAASGFTFPWYRVHWVRQAPGKGLEWVSSIRSSGGFPYYNNHVKGRFTISRDNSKNTLYLQMNSLRAEDTAVYYCARFYDSFFDIWGQGTMVTVSS

SEQ ID NO: 73 - protein, 117 aa, synthetic construct
EVQLLESGGGLVQPGGSLRLSCAASGFTFPWYRVHWVRQAPGKGLEWVSSIRSSGGFPMYNPHVKGRFTISRDNSKNTLYLQMNSLRAEDTAVYYCARFYDSFFDIWGQGTMVTVSS

SEQ ID NO: 74 - protein, 117 aa, synthetic construct
EVQLLESGGGLVQPGGSLRLSCAASGFTFPWYRVHWVRQAPGKGLEWVSSIRSSGGFTYYNPSVKNRFTISRDNSKNTLYLQMNSLRAEDTAVYYCARFYDSFFDIWGQGTMVTVSS

SEQ ID NO: 75 - protein, 117 aa, synthetic construct
EVQLLESGGGLVQPGGSLRLSCAASGFTFPWYRVHWVRQAPGKGLEWVSSIRSSGGFTYYNPAVRGRFTISRDNSKNTLYLQMNSLRAEDTAVYYCARFYDSFFDIWGQGTMVTVSS

SEQ ID NO: 76 - protein, 117 aa, synthetic construct
EVQLLESGGGLVQPGGSLRLSCAASGFTFPWYRVHWVRQAPGKGLEWVSSIRSSGGFTYYNPKVKHRFTISRDNSKNTLYLQMNSLRAEDTAVYYCARFYDSFFDIWGQGTMVTVSS

SEQ ID NO: 77 - protein, 117 aa, synthetic construct
EVQLLESGGGLVQPGGSLRLSCAASGFTFPWYRVHWVRQAPGKGLEWVSSIRSSGGFTYYNSEVKGRFTISRDNSKNTLYLQMNSLRAEDTAVYYCARFYDSFFDIWGQGTMVTVSS

SEQ ID NO: 78 - protein, 117 aa, synthetic construct
EVQLLESGGGLVQPGGSLRLSCAASGFTFPWYRVHWVRQAPGKGLEWVSSIRSSGGFTYYTPSVRGRFTISRDNSKNTLYLQMNSLRAEDTAVYYCARFYDSFFDIWGQGTMVTVSS

SEQ ID NO: 79 - protein, 117 aa, synthetic construct
EVQLLESGGGLVQPGGSLRLSCAASGFTFPWYRVHWVRQAPGKGLEWVSSIRSSGGFTYYNPSVMGRFTISRDNSKNTLYLQMNSLRAEDTAVYYCARFYDSFFDIWGQGTMVTVSS

SEQ ID NO: 80 - protein, 117 aa, synthetic construct
EVQLLESGGGLVQPGGSLRLSCAASGFTFFWFHHVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 81 - protein, 117 aa, synthetic construct
EVQLLESGGGLVQPGGSLRLSCAASGFTFYWYHMIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 82 - protein, 117 aa, synthetic construct
EVQLLESGGGLVQPGGSLRLSCAASGFTFNWFQHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 83 - protein, 117 aa, synthetic construct
EVQLLESGGGLVQPGGSLRLSCAASGFTFPWFHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 84 - protein, 117 aa, synthetic construct
EVQLLESGGGLVQPGGSLRLSCAASGFTFQWYHYIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 85 - protein, 117 aa, synthetic construct
EVQLLESGGGLVQPGGSLRLSCAASGFTFQEFHTIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 86 - protein, 117 aa, synthetic construct
EVQLLESGGGLVQPGGSLRLSCAASGFTFFWFNTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 87 - protein, 117 aa, synthetic construct
EVQLLESGGGLVQPGGSLRLSCAASGFTFQWFNIVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 88 - protein, 117 aa, synthetic construct
EVQLLESGGGLVQPGGSLRLSCAASGFTFPFYHKVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 89 - protein, 117 aa, synthetic construct
EVQLLESGGGLVQPGGSLRLSCAASGFTFPGYVWIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 90 - protein, 117 aa, synthetic construct
EVQLLESGGGLVQPGGSLRLSCAASGFTFQYFNVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 91 - protein, 117 aa, synthetic construct
EVQLLESGGGLVQPGGSLRLSCAASGFTFQHYHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 92 - protein, 117 aa, synthetic construct
EVQLLESGGGLVQPGGSLRLSCAASGFTFQWYHVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 93 - protein, 117 aa, synthetic construct
EVQLLESGGGLVQPGGSLRLSCAASGFTFHHYHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 94 - protein, 117 aa, synthetic construct
EVQLLESGGGLVQPGGSLRLSCAASGFTFHHFHVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 95 - protein, 117 aa, synthetic construct
EVQLLESGGGLVQPGGSLRLSCAASGFTFPWFHFVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 96 - protein, 117 aa, synthetic construct
EVQLLESGGGLVQPGGSLRLSCAASGFTFEHYHTIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 97 - protein, 117 aa, synthetic construct
EVQLLESGGGLVQPGGSLRLSCAASGFTFQWFHYIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 98 - protein, 117 aa, synthetic construct
EVQLLESGGGLVQPGGSLRLSCAASGFTFQHFHWVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 99 - protein, 117 aa, synthetic construct
EVQLLESGGGLVQPGGSLRLSCAASGFTFPYYHMIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 100 - protein, 117 aa, synthetic construct
EVQLLESGGGLVQPGGSLRLSCAASGFTFMWYHHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 101 - protein, 117 aa, synthetic construct
EVQLLESGGGLVQPGGSLRLSCAASGFTFVWYHHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 102 - protein, 117 aa, synthetic construct; identical to SEQ ID NO: 95

SEQ ID NO: 103 - protein, 117 aa, synthetic construct
EVQLLESGGGLVQPGGSLRLSCAASGFTFPYYHQVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 104 - protein, 117 aa, synthetic construct
EVQLLESGGGLVQPGGSLRLSCAASGFTFPWFVMVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 105 - protein, 117 aa, synthetic construct
EVQLLESGGGLVQPGGSLRLSCAASRTSFMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 106 - protein, 117 aa, synthetic construct
EVQLLESGGGLVQPGGSLRLSCAASRGAPMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 107 - protein, 117 aa, synthetic construct
EVQLLESGGGLVQPGGSLRLSCAASDVDFGWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 108 - protein, 117 aa, synthetic construct
EVQLLESGGGLVQPGGSLRLSCAASAGFFEYYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 109 - protein, 117 aa, synthetic construct
EVQLLESGGGLVQPGGSLRLSCAASPRWEWNYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 110 - protein, 117 aa, synthetic construct
EVQLLESGGGLVQPGGSLRLSCAASAYSEMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 111 - protein, 117 aa, synthetic construct
EVQLLESGGGLVQPGGSLRLSCAASGFTFSHYHMLWVRQAPGKGLEWVSSINGRGRVTHVAQEVKGRFTISRDNSKNTLYLQMNSLRAEDTAVYYCARFYDSFFDIWGQGTMVTVSS

SEQ ID NO: 112 - protein, 117 aa, synthetic construct
EVQLLESGGGLVQPGGSLRLSCAASGFTFSHYHMLWVRQAPGKGLEWVSSINGRGRVPQMAQEVKGRFTISRDNSKNTLYLQMNSLRAEDTAVYYCARFYDSFFDIWGQGTMVTVSS

SEQ ID NO: 113 - protein, 117 aa, synthetic construct
EVQLLESGGGLVQPGGSLRLSCAASGFTFSHYHMLWVRQAPGKGLEWVSSINGRGRVTYYQWPMNKRFTISRDNSKNTLYLQMNSLRAEDTAVYYCARFYDSFFDIWGQGTMVTVSS

SEQ ID NO: 114 - protein, 117 aa, synthetic construct
EVQLLESGGGLVQPGGSLRLSCAASGFTFSHYHMLWVRQAPGKGLEWVSSINGRGRVTYYNPAWKKRFTISRDNSKNTLYLQMNSLRAEDTAVYYCARFYDSFFDIWGQGTMVTVSS

SEQ ID NO: 115 - protein, 117 aa, synthetic construct
EVQLLESGGGLVQPGGSLRLSCAASGFTFSHYHMLWVRQAPGKGLEWVSSINGRGRVTYYARDHKKRFTISRDNSKNTLYLQMNSLRAEDTAVYYCARFYDSFFDIWGQGTMVTVSS

SEQ ID NO: 116 - protein, 117 aa, synthetic construct
EVQLLESGGGLVQPGGSLRLSCAASGFTFSHYHMLWVRQAPGKGLEWVSSINGRGRVTYYALEYKKRFTISRDNSKNTLYLQMNSLRAEDTAVYYCARFYDSFFDIWGQGTMVTVSS

SEQ ID NO: 117 - protein, 428 aa, Homo sapiens
WERSLAGAEETIPLQTLRCYADYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 118 - protein, 428 aa, Homo sapiens
WERSLAGAEETIPLQTLRCYNAYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 119 - protein, 428 aa, Homo sapiens
WERSLAGAEETIPLQTLRCYNDATSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 120 - protein, 428 aa, Homo sapiens
WERSLAGAEETIPLQTLRCYNDYASHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 121 - protein, 428 aa, Homo sapiens
WERSLAGAEETIPLQTLRCYNDYTAHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 122 - protein, 428 aa, Homo sapiens
WERSLAGAEETIPLQTLRCYNDYTSAITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 123 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQAFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 124 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSAVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 125 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFAVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 126 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVATDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 127 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVADVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 128 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTAVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 129 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDADYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 130 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVAIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 131 - protein, 444 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNAQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 132 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIAMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 133 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQAAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 134 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMAMRYEHIDHTFEIQYRKDTATWKDSKTETLQNAHSMALPALEPSTRYWARVRVRTSRTGYNGIWSEWSEARSWDTESHHHHHH

SEQ ID NO: 135 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKARYEHIDHTFEIQYRKDTATWKDSKTETLQNAHSMALPALEPSTRYWARVRVRTSRTGYNGIWSEWSEARSWDTESHHHHHH

SEQ ID NO: 136 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMAYEHIDHTFEIQYRKDTATWKDSKTETLQNAHSMALPALEPSTRYWARVRVRTSRTGYNGIWSEWSEARSWDTESHHHHHH

SEQ ID NO: 137 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRAEHIDHTFEIQYRKDTATWKDSKTETLQNAHSMALPALEPSTRYWARVRVRTSRTGYNGIWSEWSEARSWDTESHHHHHH

SEQ ID NO: 138 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYAHIDHTFEIQYRKDTATWKDSKTETLQNAHSMALPALEPSTRYWARVRVRTSRTGYNGIWSEWSEARSWDTESHHHHHH

SEQ ID NO: 139 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEAIDHTFEIQYRKDTATWKDSKTETLQNAHSMALPALEPSTRYWARVRVRTSRTGYNGIWSEWSEARSWDTESHHHHHH

SEQ ID NO: 140 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHADHTFEIQYRKDTATWKDSKTETLQNAHSMALPALEPSTRYWARVRVRTSRTGYNGIWSEWSEARSWDTESHHHHHH

SEQ ID NO: 141 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 142 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSATGYNGIWSEWSEARSWDTESHHHHHH

SEQ ID NO: 143 - protein, 428 aa, Homo sapiens; identical to SEQ ID NO: 142

SEQ ID NO: 144 - protein, 444 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTAYNGIWSEWSEARSWDTESHHHHHH

SEQ ID NO: 145 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGANGIWSEWSEARSWDTESHHHHHH

SEQ ID NO: 146 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYAGIWSEWSEARSWDTESHHHHHH

SEQ ID NO: 147 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNAIWSEWSEARSWDTESHHHHHH

SEQ ID NO: 148 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGAWSEWSEARSWDTESHHHHHH

SEQ ID NO: 149 - DNA, 41 bp, synthetic construct
  source 1..41: mol_type = other DNA
  misc_difference 18..23: note = n is any nucleotide provided that a codon comprising the n encoding an amino acid other than cysteine
gtcgtgcaagccagggtnnnnnntggtatcagcagaaaccg

SEQ ID NO: 150 - DNA, 45 bp, synthetic construct
  source 1..45: mol_type = other DNA
  misc_difference 22..27: note = n is any nucleotide provided that a codon comprising the n encoding an amino acid other than cysteine
  misc_difference 32..37: note = n is any nucleotide provided that a codon comprising the n encoding an amino acid other than cysteine
gattttgcaacctattattgcnnnnnnccgattacctttggtcag

SEQ ID NO: 151 - DNA, 42 bp, synthetic construct
  source 1..42: mol_type = other DNA
  misc_difference 20..25: note = n is any nucleotide provided that a codon comprising the n encoding an amino acid other than cysteine
  misc_difference 21..26: note = n is any nucleotide provided that a codon comprising the n encoding an amino acid other than cysteine
ctattattgccagcaggcannnnnntttggtcagggcacccg

SEQ ID NO: 152 - DNA, 42 bp, synthetic construct
  source 1..42: mol_type = other DNA
  misc_difference 20..25: note = n is any nucleotide provided that a codon comprising the n encoding an amino acid other than cysteine
agcaagcggttttacctttnnnnnntgggttcgtcaggcacc

SEQ ID NO: 153 - DNA, 44 bp, synthetic construct
  source 1..44: mol_type = other DNA
  misc_difference 20..25: note = n is any nucleotide provided that a codon comprising the n encoding an amino acid other than cysteine
ggaatgggttagcagcattnnnnnnacctattatgccgatagcg

SEQ ID NO: 154 - DNA, 43 bp, synthetic construct
  source 1..43: mol_type = other DNA
  misc_difference 20..25: note = n is any nucleotide provided that a codon comprising the n encoding an amino acid other than cysteine
agtttattattgcgcacgcnnnnnngatatttggggtcagggt

SEQ ID NO: 155 - DNA, 44 bp, synthetic construct
  source 1..44: mol_type = other DNA
  misc_difference 21..26: note = n is any nucleotide provided that a codon comprising the n encoding an amino acid other than cysteine
attattgcgcacgcttttatnnnnnntggggtcagggtacaatg

SEQ ID NO: 156 - DNA, 44 bp, synthetic construct; identical to SEQ ID NO: 153
  source 1..44: mol_type = other DNA
  misc_difference 20..25: note = n is any nucleotide provided that a codon comprising the n encoding an amino acid other than cysteine

SEQ ID NO: 157 - DNA, 44 bp, synthetic construct
  source 1..44: mol_type = other DNA
  misc_difference 19..24: note = n is any nucleotide provided that a codon comprising the n encoding an amino acid other than cysteine
cgtagcagcggtggttttnnnnnngtgaaaggtcgttttaccat

SEQ ID NO: 158 - DNA, 45 bp, synthetic construct
  source 1..45: mol_type = other DNA
  misc_difference 21..26: note = n is any nucleotide provided that a codon comprising the n encoding an amino acid other than cysteine
gcggtggttttacctattatnnnnnncgttttaccattagccgtg

SEQ ID NO: 159 - DNA, 42 bp, synthetic construct; identical to SEQ ID NO: 152
  source 1..42: mol_type = other DNA
  misc_difference 20..25: note = n is any nucleotide provided that a codon comprising the n encoding an amino acid other than cysteine

SEQ ID NO: 160 - DNA, 42 bp, synthetic construct
  source 1..42: mol_type = other DNA
  misc_difference 18..23: note = n is any nucleotide provided that a codon comprising the n encoding an amino acid other than cysteine
tgagctgtgcagcaagcnnnnnntatcatatgctgtgggttc

SEQ ID NO: 161 - DNA, 43 bp, synthetic construct
  source 1..43: mol_type = other DNA
  misc_difference 19..24: note = n is any nucleotide provided that a codon comprising the n encoding an amino acid other than cysteine
aatggtcgtggtcgtgttnnnnnngtgaaaggtcgttttacca

SEQ ID NO: 162 - DNA, 45 bp, synthetic construct
  source 1..45: mol_type = other DNA
  misc_difference 21..26: note = n is any nucleotide provided that a codon comprising the n encoding an amino acid other than cysteine
gtggtcgtgttacctattatnnnnnncgttttaccattagccgtg

SEQ ID NO: 163 - protein, 117 aa, synthetic construct
EVQLLESGGGLVQPGGSLRLSCAASGFTFXXXXXXWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 164 - protein, 117 aa, synthetic construct
EVQLLESGGGLVQPGGSLRLSCAASGFTFSHYHMLWVRQAPGKGLEWVSSIXXXXXXTYYADSVKGRFTISRDNSKNTLYLQMNSLRAEDTAVYYCARFYDSFFDIWGQGTMVTVSS

SEQ ID NO: 165 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARXXXXXXDIWGQGTMVTVSS

SEQ ID NO: 166 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYXXXXXXWGQGTMVTVSS

SEQ ID NO: 167 - protein, 112 aa, synthetic construct
DIQMTQSPSSVSASVGDRVTITCRASQGXXXXXXWYQQKPGKAPKLLIYAASSLQSGVPSRFSGSGSGTDFTLTISSLQPEDFATYYCQQANSFPITFGQGTRLEIKRTVAA

SEQ ID NO: 168 - protein, 112 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCXXXXXXPITFGQGTRLEIKRTVAA

SEQ ID NO: 169 - protein, 112 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAXXXXXXFGQGTRLEIKRTVAA

SEQ ID NO: 170 - protein, 117 aa, synthetic construct
EVQLLESGGGLVQPGGSLRLSCAASGFTFPWYRVHWVRQAPGKGLEWVSSIXXXXXXTYYADSVKGRFTISRDNSKNTLYLQMNSLRAEDTAVYYCARFYDSFFDIWGQGTMVTVSS

SEQ ID NO: 171 - protein, 117 aa, synthetic construct
EVQLLESGGGLVQPGGSLRLSCAASGFTFPWYRVHWVRQAPGKGLEWVSSIRSSGGFXXXXXXVKGRFTISRDNSKNTLYLQMNSLRAEDTAVYYCARFYDSFFDIWGQGTMVTVSS

SEQ ID NO: 172 - protein, 117 aa, synthetic construct
EVQLLESGGGLVQPGGSLRLSCAASGFTFPWYRVHWVRQAPGKGLEWVSSIRSSGGFTYYXXXXXXRFTISRDNSKNTLYLQMNSLRAEDTAVYYCARFYDSFFDIWGQGTMVTVSS

SEQ ID NO: 173 - protein, 117 aa, synthetic construct
EVQLLESGGGLVQPGGSLRLSCAASGFTFXXXXXXWVRQAPGKGLEWVSSINGRGRVTYYADSVKGRFTISRDNSKNTLYLQMNSLRAEDTAVYYCARFYDSFFDIWGQGTMVTVSS

SEQ ID NO: 174 - protein, 117 aa, synthetic construct
EVQLLESGGGLVQPGGSLRLSCAASXXXXXXYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 175 - protein, 117 aa, synthetic construct
EVQLLESGGGLVQPGGSLRLSCAASGFTFSHYHMLWVRQAPGKGLEWVSSINGRGRVXXXXXXVKGRFTISRDNSKNTLYLQMNSLRAEDTAVYYCARFYDSFFDIWGQGTMVTVSS

SEQ ID NO: 176 - protein, 117 aa, synthetic construct
EVQLLESGGGLVQPGGSLRLSCAASGFTFSHYHMLWVRQAPGKGLEWVSSINGRGRVTYYXXXXXXRFTISRDNSKNTLYLQMNSLRAEDTAVYYCARFYDSFFDIWGQGTMVTVSS

SEQ ID NO: 177 - protein, 108 aa, synthetic construct
  VARIANT 29: note = X is I, Q, L or V
  VARIANT 30: note = X is S or R
  VARIANT 31: note = X is S or P
  VARIANT 32: note = X is W or F
  VARIANT 33: note = X is L, M, V or I
  VARIANT 34: note = X is A, S, N or D
  VARIANT 89: note = X is Q or G
  VARIANT 90: note = X is Q, K or I
  VARIANT 91: note = X is A or P
  VARIANT 92: note = X is N, V or I
  VARIANT 93: note = X is S, L or F
  VARIANT 94: note = X is F, D or G
DIQMTQSPSSVSASVGDRVTITCRASQGXXXXXXWYQQKPGKAPKLLIYAASSLQSGVPSRFSGSGSGTDFTLTISSLQPEDFATYYCXXXXXXPITFGQGTRLEIKR

SEQ ID NO: 178 - protein, 11 aa, synthetic construct
  VARIANT 6: note = X is I, Q, L or V
  VARIANT 7: note = X is S or R
  VARIANT 8: note = X is S or P
  VARIANT 9: note = X is W or F
  VARIANT 10: note = X is L, M, V or I
  VARIANT 11: note = X is A, S, N or D
RASQGXXXXXX

SEQ ID NO: 179 - skipped

SEQ ID NO: 180 - protein, 117 aa, synthetic construct
  VARIANT 30: note = X is S, N, M, Q, E, P, I, T or V
  VARIANT 31: note = X is H or W
  VARIANT 33: note = X is H or R
  VARIANT 34: note = X is M or V
  VARIANT 35: note = X is L or H
  VARIANT 52: note = X is R, S, N or G
  VARIANT 53: note = X is S or G
  VARIANT 54: note = X is S, A, F, D, M, R, Y or K
  VARIANT 55: note = X is G, Q, V, H, K, M or I
  VARIANT 56: note = X is G, W, R, F, W, A or I
  VARIANT 57: note = X is F, S, T, P, N, Q or V
  VARIANT 101: note = X is D or S
  VARIANT 102: note = X is S or D
  VARIANT 103: note = X is F, H or N
  VARIANT 105: note = X is D, R, S, K, N or A
  VARIANT 106: note = X is I, E or P
EVQLLESGGGLVQPGGSLRLSCAASGFTFXXYXXXWVRQAPGKGLEWVSSIXXXXXXTYYADSVKGRFTISRDNSKNTLYLQMNSLRAEDTAVYYCARFYXXXFXXWGQGTMVTVSS

SEQ ID NO: 181 - protein, 10 aa, synthetic construct
  VARIANT 5: note = X is S, N, M, Q, E, P, I, T or V
  VARIANT 6: note = X is H or W
  VARIANT 8: note = X is H or R
  VARIANT 9: note = X is M or V
  VARIANT 10: note = X is L or H
GFTFXXYXXX

SEQ ID NO: 182 - protein, 17 aa, synthetic construct
  VARIANT 3: note = X is R, S, N or G
  VARIANT 4: note = X is S or G
  VARIANT 5: note = X is S, A, F, D, M, R, Y or K
  VARIANT 6: note = X is G, Q, V, H, K, M or I
  VARIANT 7: note = X is G, W, R, F, W, A or I
  VARIANT 8: note = X is F, S, T, P, N, Q or V
SIXXXXXXTYYADSVKG

SEQ ID NO: 183 - skipped

SEQ ID NO: 184 - protein, 117 aa, synthetic construct
  VARIANT 52: note = X is R, H, Q or K
  VARIANT 53: note = X is S, T, H or N
  VARIANT 54: note = X is S, H, G, Q, D or E
  VARIANT 55: note = X is G, R, S, Y or N
  VARIANT 56: note = X is G, H, N, Q, K, T, V, R, P or D
  VARIANT 57: note = X is F, I, S, W, V or Y
  VARIANT 58: note = X is T or P
  VARIANT 59: note = X is Y, H, F or M
  VARIANT 61: note = X is A, H, N, S or T
  VARIANT 62: note = X is D, Q, Y, R, H, P, L, S or N
  VARIANT 63: note = X is S, K, R, V, F, H, A or E
  VARIANT 65: note = X is K, R or M
  VARIANT 66: note = X is G, N or H
EVQLLESGGGLVQPGGSLRLSCAASGFTFPWYRVHWVRQAPGKGLEWVSSIXXXXXXXXYXXXVXXRFTISRDNSKNTLYLQMNSLRAEDTAVYYCARFYDSFFDIWGQGTMVTVSS

SEQ ID NO: 185 - protein, 10 aa, synthetic construct
GFTFPWYRVH

SEQ ID NO: 186 - protein, 17 aa, synthetic construct
  VARIANT 3: note = X is R, H, Q or K
  VARIANT 4: note = X is S, T, H or N
  VARIANT 5: note = X is S, H, G, Q, D or E
  VARIANT 6: note = X is G, R, S, Y or N
  VARIANT 7: note = X is G, H, N, Q, K, T, V, R, P or D
  VARIANT 8: note = X is F, I, S, W, V or Y
  VARIANT 9: note = X is T or P
  VARIANT 10: note = X is Y, H , F or M
  VARIANT 12: note = X is A, H, N, S or T
  VARIANT 13: note = X is D, Q, Y, R, H, P, L, S or N
  VARIANT 14: note = X is S, K, R, V, F, H, A or E
  VARIANT 16: note = X is K, R or M
  VARIANT 17: note = X is G, N or H
SIXXXXXXXXYXXXVXX

SEQ ID NO: 187 - protein, 8 aa, synthetic construct
FYDSFFDI

SEQ ID NO: 188 - protein, 117 aa, synthetic construct
  VARIANT 26: note = X is G, R, D, A or P
  VARIANT 27: note = X is F, G, V, R or Y
  VARIANT 28: note = X is T, S, A, P, F or W
  VARIANT 29: note = X is F, P or E
  VARIANT 30: note = X is S, F, Y, N, P, Q, H, E, M, V, G, W or M
  VARIANT 31: note = X is H, W, E, F, G, Y or N
  VARIANT 32: note = X is Y or F
  VARIANT 33: note = X is H, M, Q, N or V
  VARIANT 34: note = X is M, H, T, Y, I, K, W, V, F or Q
  VARIANT 35: note = X is L, V or I
  VARIANT 59: note = X is Y, H or Q
  VARIANT 60: note = X is Y, V or M
  VARIANT 61: note = X is A, Q or N
  VARIANT 62: note = X is D, Q, W, P, R or L
  VARIANT 63: note = X is S, E, P, A or D
  VARIANT 64: note = X is V, M, W, H or Y
  VARIANT 65: note = X is K or N
  VARIANT 66: note = X is G or K
EVQLLESGGGLVQPGGSLRLSCAASXXXXXXXXXXWVRQAPGKGLEWVSSINGRGRVTXXXXXXXXRFTISRDNSKNTLYLQMNSLRAEDTAVYYCARFYDSFFDIWGQGTMVTVSS

SEQ ID NO: 189 - skipped

SEQ ID NO: 190 - protein, 17 aa, synthetic construct
  VARIANT 10: note = X is Y, H or Q
  VARIANT 11: note = X is Y, V or M
  VARIANT 12: note = X is A, Q or N
  VARIANT 13: note = X is D, Q, W, P, R or L
  VARIANT 14: note = X is S, E, P, A or D
  VARIANT 15: note = X is V, M, W, H or Y
  VARIANT 16: note = X is K or N
  VARIANT 17: note = X is G or K
SINGRGRVTXXXXXXXX

SEQ ID NO: 191 - protein, 8 aa, synthetic construct
FYDSFFDI

SEQ ID NO: 192 - protein, 428 aa, Homo sapiens; identical to SEQ ID NO: 141

SEQ ID NO: 193 - protein, 117 aa, synthetic construct; identical to SEQ ID NO: 184

SEQ ID NO: 194 - protein, 444 aa, synthetic construct
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - protein, 214 aa, synthetic construct
DIQMTQSPSSVSASVGDRVTITCRASQGLRPFVNWYQQKPGKAPKLLIYAASSLQSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - protein, 104 aa, synthetic construct
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0188173 A1
(43) Pub. Date: Jun. 12, 2025

(54) Method of Treating an Eosinophil-Associated Disease

(71) Applicant: CSL Innovation Pty Ltd, Melbourne, Victoria (AU)

(72) Inventors: Steven Bozinovski, Melbourne (AU); Catherine Mary Owczarek, Melbourne (AU); Hao Wang, Melbourne (AU)

(73) Assignee: CSL Innovation Pty Ltd, Melbourne, Victoria (AU)

(21) Appl. No.: 18/846,527

(22) PCT Filed: Mar. 16, 2023
(86) PCT No.: PCT/AU2023/050186

§ 371 Date: Sep. 12, 2024

(30) Foreign Application Priority Data
Mar. 16, 2022 (AU) 2022900636

(51) Int. Cl. C07K 16/28; A61K 39/00; A61P 11/00; A61P 29/00; G01N 33/50

(52) U.S. Cl. CPC C07K 16/2866; A61P 11/00; A61P 29/00; G01N 33/5094; A61K 2039/505; C07K 2317/524; C07K 2317/526; C07K 2317/55; C07K 2317/565; C07K 2317/622; C07K 2317/76

(57) ABSTRACT

The present disclosure relates to methods of treating a subject suffering from an eosinophil-associated chronic obstructive pulmonary disease (COPD) or asthma-COPD overlap (ACO) using compounds that bind to CD131 and inhibit signalling of interleukin (IL) 3, IL-5 and granulocyte-macrophage colony stimulating factor (GM-CSF).

### RELATED APPLICATION DATA

The present application claims priority from Australian Patent Application No. 2022900636 filed 16 Mar. 2022 entitled “Method of treating an eosinophil-associated disease” the entire contents of which are hereby incorporated by reference.

### SEQUENCE LISTING

The present application is filed together with a Sequence Listing in electronic form. The entire contents of the Sequence Listing are hereby incorporated by reference.

### FIELD

The present disclosure relates to methods of treating a subject suffering from an eosinophil-associated chronic obstructive pulmonary disease (COPD) or asthma COPD overlap (ACO) using compounds that bind to CD131 and inhibit signalling of interleukin (IL) 3, IL-5 and granulocyte-macrophage colony stimulating factor (GM-CSF).

### BACKGROUND

Chronic obstructive pulmonary disease (COPD) is a chronic inflammatory lung disease that is characterised by persistent and progressive airway obstruction. Recognised risk factors for the development of COPD include long-term exposure to noxious particles, such as cigarette smoke.

An important driver of COPD pathophysiology is chronic lung inflammation, orchestrated by key leukocyte populations including neutrophils and macrophages that accumulate around the airways as the severity of the disease progresses. Airway neutrophilia is a hallmark immunological feature of COPD, where increased sputum neutrophil numbers correlate with lung function decline in COPD patients. There is also a significant subset of COPD patients (10-40%) where eosinophilic inflammation is a prominent immunological feature.

Despite decades of research, there is no cure for COPD, with the associated mortality rate remaining high. Current therapies, such as corticosteroids, oxygen therapy and bronchodilator inhalers, only aim to control symptoms and minimise further damage. More recently, several monoclonal antibodies have been developed (e.g., mepolizumab and benralizumab), however their clinical efficacy has been limited, possibly due to these antibodies targeting only individual cytokines associated with the inflammatory response.

Thus, there remains a need for improved therapies for treating COPD and/or preventing or delaying disease progression.

### SUMMARY

In producing the present invention, the inventors identified CD131 (the β common receptor) as a potential target for pharmacological intervention of esosinophilic inflammation associated with smoking-related chronic obstructive pulmonary disease (COPD). The inventors found that antibodies that bind to CD131, and inhibit signalling of interleukin (IL) 3, IL-5 and granulocyte-macrophage colony stimulating factor (GM-CSF), successfully reduced several measures of lung inflammation in an animal model of acute cigarette exposure.

The findings by the inventors provide the basis for methods of treating eosinophilic inflammation (i.e., eosinophilia) associated with smoking-related COPD in a subject.

Accordingly, the present disclosure provides a method of treating eosinophilic inflammation (i.e., eosinophilia) associated with a smoking-related COPD in a subject comprising administering to the subject a compound that binds to or specifically binds to CD131 and neutralises signalling by IL-3, IL-5 and GM-CSF in a subject.

The present disclosure also provides a compound that binds to or specifically binds to CD131 and neutralises signalling by IL-3, IL-5 and GM-CSF for use in treating eosinophilic inflammation (i.e., eosinophilia) associated with a smoking-related COPD in a subject. The present disclosure also provides use of a compound that binds to or specifically binds to CD131 and neutralises signalling by IL-3, IL-5 and GM-CSF in the manufacture of a medicament for treating eosinophilic inflammation (i.e., eosinophilia) associated with a smoking-related COPD in a subject.

The findings by the inventors also provide the basis for methods of treating an eosinophil-associated, smoking-related COPD in a subject.

The present disclosure also provides a method of treating an eosinophil-associated, smoking-related COPD, the method comprising administering to the subject a compound that binds to or specifically binds to CD131 and neutralises signalling by IL-3, IL-5 and GM-CSF in a subject.

The present disclosure also provides a compound that binds to or specifically binds to CD131 and neutralises signalling by IL-3, IL-5 and GM-CSF for use in treating an eosinophil-associated, smoking-related COPD in a subject. The present disclosure also provides use of a compound that binds to or specifically binds to CD131 and neutralises signalling by IL-3, IL-5 and GM-CSF in the manufacture of a medicament for treating an eosinophil-associated, smoking-related COPD in a subject.

The present disclosure also provides a method of depleting and/or reducing eosinophils in a subject suffering from COPD, the method comprising administering to the subject a compound that binds to or specifically binds to CD131 and neutralises signalling by IL-3, IL-5 and GM-CSF in a subject. For example, the disclosure provides a method of depleting eosinophils in a subject suffering from COPD. In another example, the disclosure provides a method of reducing eosinophils in a subject suffering from COPD. In a further example, the disclosure provides a method of depleting and reducing eosinophils in a subject suffering from COPD.

The present disclosure also provides a compound that binds to or specifically binds to CD131 and neutralises signalling by IL-3, IL-5 and GM-CSF for use in depleting and/or reducing eosinophils in a subject suffering from COPD. The present disclosure further provides a compound that binds to or specifically binds to CD131 and neutralises signalling by IL-3, IL-5 and GM-CSF in the manufacture of a medicament for depleting and/or reducing eosinophils in a subject suffering from COPD.

The present disclosure also provides a method of treating eosinophilia in a subject suffering from COPD, the method comprising administering to the subject a compound that binds to or specifically binds to CD131 and neutralises signalling by IL-3, IL-5 and GM-CSF in a subject.

The present disclosure also provides a compound that binds to or specifically binds to CD131 and neutralises signalling by IL-3, IL-5 and GM-CSF for use in treating eosinophilia in a subject suffering from COPD. The present disclosure further provides a compound that binds to or specifically binds to CD131 and neutralises signalling by IL-3, IL-5 and GM-CSF in the manufacture of a medicament for treating eosinophilia in a subject suffering from COPD.

The present disclosure further provides a method of treating an eosinophil-associated COPD, the method comprising administering to the subject a compound that binds to or specifically binds to CD131 and neutralises signalling by IL-3, IL-5 and GM-CSF in a subject.

The present disclosure also provides a compound that binds to or specifically binds to CD131 and neutralises signalling by IL-3, IL-5 and GM-CSF for use in treating an eosinophil-associated COPD in a subject. The present disclosure further provides a compound that binds to or specifically binds to CD131 and neutralises signalling by IL-3, IL-5 and GM-CSF in the manufacture of a medicament for treating an eosinophil-associated COPD in a subject.

In one example, the subject is suffering from COPD (i.e., the subject is in need of treatment). For example, the subject is suffering from clinically diagnosed COPD.

In one example, the COPD is early COPD, or Stage I COPD.

In one example, the COPD is moderate COPD, or Stage II COPD.

In one example, the COPD is severe COPD, or Stage III COPD.

In one example, the COPD is very severe COPD, Stage IV COPD, or end-stage COPD.

In one example, the severity of COPD is categorized according to the Global Initiative for Chronic Obstructive Lung Disease (GOLD) 2023 guidelines (published Nov. 14, 2022).

The severity of COPD can be categorized based on the forced expiratory volume (FEV-1) and/or according to the Global Initiative for Chronic Obstructive Lung Disease (GOLD) system as follows:
GOLD 1: Mild/early COPD: FEV-1≥80%
GOLD 2: Moderate COPD: FEV-1 50-79%
GOLD 3: Severe COPD: FEV-1 30-49%
GOLD 4: Very severe COPD: FEV-1≤30%

In one example, the severity of the COPD can be categorized according to the GOLD 2023 guidelines based on symptoms and previous history of exacerbations. Symptoms are assessed using the Modified British Medical Research Council (mMRC) or COPD assessment test (CAT) scale as follows:
Group A: low risk (0-1 moderate exacerbation per year, not requiring hospitalization) and fewer symptoms (mMRC 0-1 or CAT <10)
Group B: low risk (0-1 moderate exacerbation per year, not requiring hospitalization) and more symptoms (mMRC≥2 or CAT≥10)
Group E: high risk (≥2 moderate exacerbations per year, or one or more exacerbation requiring hospitalization) irrespective of mMRC and/or CAT scores.

It will be apparent to the skilled person that in the GOLD 2023 guidelines former Groups C and D have been merged into a single Group E.

In one example, the subject is in Group A, according to the GOLD 2023 guidelines.

In one example, the subject is in Group B, according to the GOLD 2023 guidelines.

In one example, the subject is in Group E, according to the GOLD 2023 guidelines.

In one example, the subject is receiving, or has received treatment with a long-acting muscarinic antagonist (LAMA) and a long-acting beta-agonist (LABA). In one example, the subject is receiving, or has received treatment with along-acting muscarinic antagonist (LAMA), a long-acting beta-agonist (LABA) and inhaled corticosteroid (ICS).

In one example, the subject has exacerbations despite receiving, or having received treatment with a long-acting muscarinic antagonist (LAMA) and a long-acting beta-agonist (LABA).

In one example, the subject has exacerbations despite receiving, or having received treatment with a long-acting muscarinic antagonist (LAMA) and a long-acting beta-agonist (LABA) and has a blood eosinophil count of <300 cells per μL. For example, the subject has a blood eosinophil count of <100 cells per μL.

In one example, the subject has exacerbations despite receiving, or having received treatment with a long-acting muscarinic antagonist (LAMA), a long-acting beta-agonist (LABA) and inhaled corticosteroid (ICS).

Advantageously, the methods of the present disclosure can, in addition to treatment of eosinophil-associated COPD, be used to prevent the onset of clinically diagnosed COPD. Thus, in some examples, the subject does not have clinically diagnosed COPD. For example, the subject has pre-COPD.

In another example, the methods of the present disclosure can, in addition to treatment of eosinophilia associated with COPD, be used to delay the progression of COPD. For example, the methods disclosed herein delay the progression of pre-COPD to clinically diagnosed COPD. In one example, the methods disclosed herein delay the progression of early COPD to e.g., moderate COPD.

In some examples, the subject is at risk of developing COPD. For example, the subject is at risk of developing clinically diagnosed COPD. In one example, the subject at risk has pre-COPD. Methods of identifying subjects at risk of developing COPD will be known by those skilled in the art and include those described herein.

In one example, the subject does not have pulmonary fibrosis.

In one example, the subject smokes and/or has a history of smoking and/or is exposed to second-hand smoke. For example, the subject at risk smokes and/or has a history of smoking and/or is exposed to second-hand smoke.

In one example, the subject is a heavy smoker. For example, the subject smokes at least 20 cigarettes per day.

In one example, the subject is a light smoker. For example, the subject smokes 19 cigarettes or less per day.

In one example, the subject is a current smoker. For example, the subject has smoked at least 100 cigarettes in their lifetime and has smoke all or part of a cigarette in the past 30 days.

In one example, the subject is a former smoker. For example, the subject has smoked at least 100 cigarettes in their lifetime and has not smoked all or part of a cigarette in the past 30 days.

In one example, the COPD is a smoking-related COPD.

In one example, the smoking-related COPD is caused by chronic exposure to cigarette smoke.

In one example, the compound that binds to CD131 is administered in an amount sufficient to reduce, or prevent an increase in, inflammation in the subject's lungs. In one example, the compound that binds to CD131 is administered in an amount sufficient to enhance lung function.

In one example, the compound that binds to CD131 is administered in an amount sufficient to reduce the severity of or prevent onset of one or more symptoms of the smoking-related COPD.

In one example, the compound that binds to CD131 is administered in an amount sufficient to reduce the severity of or the frequency of or prevent onset of acute exacerbations of COPD (AECOPD). For example, the compound that binds to CD131 is administered in an amount sufficient to reduce the severity of AECOPD. In another example, the compound that binds to CD131 is administered in an amount sufficient to reduce the frequency of AECOPD. In a further example, the compound that binds to CD131 is administered in an amount sufficient to prevent onset of AECOPD.

In one example, the compound that binds to CD131 is administered in an amount sufficient to prevent endotracheal intubation or death prior to endotracheal intubation. Endotracheal intubation is a process of inserting a tube, i.e., an endotracheal tube, through the mouth of the subject and into the airways so that the subject can be placed on a mechanical ventilator. Thus, the methods of the present disclosure additionally provide a method of preventing or reducing mechanical ventilation of a subject with AECOPD, the method comprising administering a compound that binds to CD131 and neutralizes signalling by IL-3, IL-5 and GM-CSF to the subject.

In one example, administration of the compound that binds to CD131 reduces, or prevents an increase in, one or more or all of the following:
(i) total amount of white blood cells in the subject's blood;
(ii) amount of monocytes in the subject's blood;
(iii) amount of neutrophils in the subject's blood;
(iv) total amount of eosinophils in the subject's blood;
(v) total amount of cells in the bronchoalveolar (BAL) fluid of the subject's lung;
(vi) macrophage accumulation in the BAL fluid of the subject's lung;
(vii) neutrophil accumulation in BAL fluid of the subject's lung;
(viii) lymphocyte accumulation in the BAL fluid of the subject's lung;
(ix) neutrophil accumulation in the subject's lung;
(x) interstitial macrophage accumulation in the subject's lung;
(xi) eosinophil accumulation in the subject's lung;
(xii) CD11b+ exudative macrophage accumulation in the subject's lung;
(xiii) total amount of eosinophils in the subject's sputum;
(xiv) total respiratory resistance of the subject;
(xv) airway resistance of the subject;
(xvi) airspace enlargement in the subject; and/or
(xvii) inflammation of the subject's lung.

In one example, administration of the compound that binds to CD131 reduces, or prevents an increase in, total amount of white blood cells in the subject's blood.

In one example, administration of the compound that binds to CD131 reduces, or prevents an increase in macrophage accumulation in the subject. In one example, the macrophages are exudative macrophages, interstitial macrophages and/or blood monocytes. For example, the exudative macrophages are CD11b+ exudative macrophages. In one example, the exudative macrophages are CD11b⁺CD11c⁺ exudative macrophages. In another example, the interstitial macrophages are CD11b⁺ interstitial macrophages. For example, the interstitial macrophages are CD11b⁺CD11c⁻ interstitial macrophages.

In one example, administration of the compound that binds to CD131 reduces, or prevents an increase in the total amount of white blood cells in the subject's blood.

In one example, administration of the compound that binds to CD131 reduces, or prevents an increase in the amount of monocytes in the subject's blood.

In one example, administration of the compound that binds to CD131 reduces, or prevents an increase in the amount of neutrophils in the subject's blood.

In one example, administration of the compound that binds to CD131 reduces, or prevents an increase in the total amount of cells in the bronchoalveolar (BAL) fluid of the subject's lung.

In one example, administration of the compound that binds to CD131 reduces, or prevents an increase in macrophage accumulation in the BAL fluid of the subject's lung. For example, CD11b+ exudative macrophage accumulation in the subject's lung.

In one example, administration of the compound that binds to CD131 reduces, or prevents an increase in neutrophil accumulation in BAL fluid of the subject's lung.

In one example, administration of the compound that binds to CD131 reduces, or prevents an increase in lymphocyte accumulation in the BAL fluid of the subject's lung.

In one example, administration of the compound that binds to CD131 reduces, or prevents an increase in neutrophil accumulation in the subject's lung.

In one example, administration of the compound that binds to CD131 reduces, or prevents an increase in interstitial macrophage accumulation in the subject's lung.

In one example, administration of the compound that binds to CD131 reduces, or prevents an increase in eosinophil accumulation in the subject's lung.

In one example, administration of the compound that binds to CD131 reduces, or prevents an increase in, total amount of eosinophils in the subject's sputum.

In one example, administration of the compound that binds to CD131 reduces, or prevents an increase in, total amount of eosinophils in the subject's blood.

In one example, administration of the compound that binds to CD131 reduces, or prevents an increase in, one or more or all of the following:
(i) lung injury score in the subject;
(ii) albumin concentration in the BAL fluid of the subject's lung;
(iii) lactate dehydrogenase (LDH) levels in the BAL fluid of the subject's lung;
(iv) amount of dsDNA in the BAL fluid of the subject's lung; and/or
(v) gelatinase levels in the BAL fluid of the subject's lung.

In one example, administration of the compound that binds to CD131 reduces, or prevents an increase in lung injury score in the subject

In one example, administration of the compound that binds to CD131 reduces, or prevents an increase in albumin concentration in the BAL fluid of the subject's lung.

In one example, administration of the compound that binds to CD131 reduces, or prevents an increase in LDH levels in the BAL fluid of the subject's lung.

In one example, administration of the compound that binds to CD131 reduces, or prevents an increase in the amount of dsDNA in the BAL fluid of the subject's lung.

In one example, administration of the compound that binds to CD131 reduces, or prevents an increase in gelatinase levels in the BAL fluid of the subject's lung.

In one example, administration of the compound that binds to CD131 reduces, or prevents an increase in nitric oxide (FeNO) in the subject's lung.

In one example, administration of the compound that binds to CD131 reduces, or prevents an increase in the level of one or more or all of the following: IL-1α, IL-17A, IL-23a, C-X-C Motif Chemokine Ligand 1 (CXC1), CXCL2, C-C Motif Chemokine Ligand 2 (CCL2), matrix metalloproteinase 12 (MMP12), Found in Inflammatory Zone 1 (Fizz1) and GM-CSF.

In one example, administration of the compound that binds to CD131 reduces, or prevents an increase in the level of one or more or all of the following: cluster of differentiation 125 (CD125), phosphorylated STAT5 (pSTAT5), CD35, CD11b, CCL17, CCL22 and CCL23. For example, administration of the compound that binds to CD131 reduces, or prevents an increase in the level of one or more or all of the following: phosphorylated STAT5 (pSTAT5), CD35, and CD11b.

In one example, administration of the compound that binds to CD131 increases, or prevents a decrease in the level of CD62L or CD63. For example, administration of the compound that binds to CD131 increases, or prevents a decrease in the level of CD62L.

In one example, administration of the compound that binds to CD131 reduces, or prevents an increase in the level of one or more or all of the following: CD63, CLL11, ECP, TL-4, 1L-13, periostin, CCL13, CCL26 and dipeptidyl-peptidase-4 (DPP4).

These biomarkers can be used to assess the efficacy of treatment or to assist in identifying a subject at risk of developing eosinophil-associated COPD.

In one example, administration of the compound that binds to CD131 reduces, or prevents an increase in the level of expression of one or more genes in the subject selected from the group consisting of IL-1α, IL-17A, IL-23a, C-X-C Motif Chemokine Ligand 1 (CXCL1), CXCL2, C-C Motif Chemokine Ligand 2 (CCL2), matrix metalloproteinase 12 (MMP-12), Found in Inflammatory Zone 1 (Fizz1) and GM-CSF.

In one example, administration of the compound that binds to CD131 reduces, or prevents an increase in the level of expression of one or more genes in the subject's lung selected from the group consisting of IL-1α, IL-17A, IL-23a, C-X-C Motif Chemokine Ligand 1 (CXCL1), CXCL2, C-C Motif Chemokine Ligand 2 (CCL2), matrix metalloproteinase 12 (MMP-12), Found in Inflammatory Zone 1 (Fizz1) and GM-CSF.

In one example, the compound that binds to CD131 is administered in an amount sufficient to reduce, or prevent an increase in, the level of chemokine gene expression in the subject. For example, the compound that binds to CD131 is administered in an amount sufficient to reduce, or prevent an increase in, the level of expression of the monocyte chemokine gene CCL2 and/or the neutrophil chemokine genes CXCL1 and CXCL2 in the subject.

In one example, the compound that binds to CD131 is administered in an amount sufficient to reduce, or prevent an increase in, the level of expression of inflammatory cytokine genes in the subject. For example, the compound binds to CD131 is administered in an amount sufficient to reduce, or prevent an increase in, the level of expression of the inflammatory cytokine gene IL-1α in the subject.

In one example, the compound that binds to CD131 is administered in an amount sufficient to reduce, or prevent an increase in, the level of expression of M2 macrophage markers in the subject. For example, the compound binds to CD131 is administered in an amount sufficient to reduce, or prevent an increase in, the level of expression of the M2 macrophage markers MMP-12 and Fizz1 in the subject.

In one example, the compound that binds to CD131 is administered in an amount sufficient to reduce, or prevent an increase in, Tₕₑₗₚₑᵣ 17 (TH17) cells in the subject. For example, the compound that binds to CD131 is administered in an amount sufficient to deplete the amount of TH cells in the subject.

In one example, the compound that binds to CD131 is administered in an amount sufficient to reduce, or prevent an increase in, TH17 mediated inflammation in the subject. For example, the compound binds to CD131 is administered in an amount sufficient to reduce, or prevent an increase in, the level of expression of the TH17 inflammatory markers IL-17A, IL-23a and GM-CSF in the subject.

Methods of measuring the level of gene expression in the subject's lung and/or blood cells will be apparent to the skilled person and/or described herein. For example, the level of gene expression is measured using real-time quantitative PCR, wherein the level of gene expression is normalised against a housekeeping gene (e.g., GAPDH).

In some examples, the levels of the above genes and/or proteins are reduced, or prevented from increasing, in the subject's lungs. In some examples, the levels of expression of the above genes are reduced, or prevented from increasing, in the subject's blood cells.

In one example, the levels of the above genes and/or proteins are reduced, or prevented from increasing, in a sample obtained from the subject. For example, the sample is a tissue and/or a fluid sample. For example, the tissue is a lung tissue sample, such as a lung biopsy sample. For example, the fluid sample is a blood sample (e.g., a whole blood sample or a serum sample), a bronchoalveolar (BAL) fluid sample or a sputum sample. It will be apparent to the skilled person that the fluid sample contains or comprises cells (e.g., inflammatory cells, such as eosinophils).

In one example, the compound that binds to CD131 of the disclosure competes with IL-3 and/or GM-CSF and/or IL-5 for binding to a cell expressing CD131 (e.g., TF-1 cells).

In one example, the compound that binds to CD131 inhibits:
(a) GM-CSF-induced proliferation of TF-1 erythroleukemic cells with an IC₅₀ of at least 460 nM; and/or
(b) IL-5-induced proliferation of TF-1 erythroleukemic cells with an IC₅₀ of at least 1600 nM; and/or
(c) IL-3-induced proliferation of TF-1 erythroleukemic cells with an IC₅₀ of at least 10 nM.

In one example, the compound that binds to CD131 inhibits GM-CSF-induced proliferation of TF-1 cells with an IC₅₀ of at least about 460 nM. For example, the IC₅₀ is at least about 300 nM or 200 nM or 100 nM. For example, the IC₅₀ is at least about 460 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.4 nM. In one example, the IC₅₀ is at least about 0.3 nM.

In one example, the compound that binds to CD131 inhibits IL-3-induced proliferation of TF-1 cells with an IC₅₀ of at least about 10 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.2 nM or at least about 0.1 nM. In one example, the IC₅₀ is at least about 0.15 nM.

In one example, the compound that binds to CD131 inhibits IL-5-induced proliferation of TF-1 cells with an IC₅₀ of at least 1600 nM. For example, the IC₅₀ is at least about 460 nM. For example, the IC₅₀ is at least about 1500 nM or about 1000 nM or about 500 nM. For example, the IC₅₀ is at least about 460 nM. For example, the IC₅₀ is at least about 300 nM or 200 nM or 100 nM. For example, the IC₅₀ is at least about 10 nM or nM or 1 nM. In one example, the IC₅₀ is at least about 5 nM. For example, the IC₅₀ is at least about 4 nM. In one example, the IC₅₀ is at least about 4.5 nM or at least about 4.6 or at least about 4.7 nM. In one example, the IC₅₀ is at least about 4.6 nM.

Methods for determining the IC₅₀ are described herein and include culturing TF-1 cells (e.g., about 1×10⁴ TF-1 cells) in the presence of the CD131-binding protein (e.g., for at least about 3 minutes or 1 hour, such as about 30 minutes) prior to adding the relevant growth factor (GM-CSF, IL-3 and/or IL-5) and culturing the cells further (e.g., for at least about 48 hours or at least about 72 hours or at least about 96 hours, e.g., for about 72 hours) and then determining cell proliferation. Cell proliferation can be determined by growing the cells in the presence of ³[H]-thymidine for about 6 hours and determining 31H1-thymidine incorporation, e.g., by liquid-scintillation counting. By determining proliferation in a variety of concentrations of the CD131-binding protein an IC₅₀ can be determined.

In one example, the compound that binds to CD131 is a CD131-binding protein. For example, a CD131-binding protein comprising an antigen binding domain of an antibody, wherein the antigen binding domain binds to or specifically binds to CD131 and neutralizes signalling by IL-3, IL-5 and GM-CSF. In some examples, the antigen binding site comprises one or more complementarity determining regions (CDRs).

Reference herein to a compound or protein or antibody that “binds to” CD131 provides literal support for a compound or protein or antibody that “binds specifically to” or “specifically binds to” CD131.

In one example, the CD131-binding protein binds to a polypeptide comprising a sequence set forth in SEQ ID NO: 194 with a K_{D} of about 100 nM or less, e.g., when the polypeptide is immobilized on a solid surface and the K_{D} is determined by surface plasmon resonance. In one example, the K_{D} is 10 nM or less, for example, 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less. In one example, the K_{D} is 0.5 nM or less. In one example, the K_{D} is 0.4 nM or less. In one example, the K_{D} is 0.3 nM or less.

In one example, the CD131-binding protein binds to a cell expressing CD131 (e.g., a neutrophil or an eosinophil or a TF-1 cell) with a K_{D} of about 10 nM or less, e.g., using a competition assay using labeled and unlabeled protein or antibody. In one example, the K_{D} is 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less.

In one example, the K_{D} is about 300 nM or less for a neutrophil.

In one example, the K_{D} is about 700 nM or less for an eosinophil.

In one example, the K_{D} is about 400 nM or less for a TF-1 cell.

In one example, the compound is a CD131-binding protein comprising an antigen binding site that:
(a) binds to or specifically binds to an epitope within site 2 of CD131 and neutralizes signaling by interleukin-3 (IL-3), interleukin-5 (IL-5) and granulocyte-macrophage colony stimulating factor (GM-CSF);
(b) binds to an epitope formed upon dimerization of two CD131 polypeptides; and
(c) binds to residues within domain 1 of a CD131 polypeptide and residues within domain 4 of another CD131 polypeptide.

In one example, the CD131-binding protein comprises an antigen binding site, wherein the antigen binding site binds to or specifically binds to an epitope within Site 2 of CD131 and neutralizes signaling by IL-3, IL-5 and GM-CSF. In this regard, the skilled artisan will be aware that Site 2 of CD131 is made up of residues from two CD131 polypeptides that form a dimer, e.g., Site 2 comprises residues within loops A-B and E-F of domain 1 of one CD131 polypeptide and residues within loops B-C and F-G of another CD131 polypeptide.

In one example, the CD131-binding protein comprises an antigen binding site, wherein the antigen binding site binds to an epitope formed upon dimerization of two CD131 polypeptides. For example, the antigen binding site binds to residues within domain 1 of a CD131 polypeptide and residues within domain 4 of another CD131 polypeptide.

In one example, the antigen binding site binds to an epitope comprising one or more of amino acids corresponding to residues 39 and/or 103 of SEQ ID NO: 1.

In one example, the antigen binding site binds to an epitope comprising one or more of amino acids corresponding to residues 338, 365, 367 and 368 of SEQ ID NO: 1.

In one example, the antigen binding site binds to an epitope formed upon dimerization of two CD131 polypeptides, wherein the epitope comprises one or more (or all) of amino acids corresponding to residues 39 and 103 of one CD131 polypeptide and residues 338, 365, 367 and 368 of the other CD131 polypeptide.

In one example, the compound is a CD131-binding protein that binds to one or more (or all) of the following mutant polypeptide(s):
(i) a mutant polypeptide comprising a sequence set forth in SEQ ID NO: 119;
(ii) a mutant polypeptide comprising a sequence set forth in SEQ ID NO: 123;
(iii) a mutant polypeptide comprising a sequence set forth in SEQ ID NO: 124;
(iv) a mutant polypeptide comprising a sequence set forth in SEQ ID NO: 135;
(v) a mutant polypeptide comprising a sequence set forth in SEQ ID NO: 131;
(vi) a mutant polypeptide comprising a sequence set forth in SEQ ID NO: 136;
(vii) a mutant polypeptide comprising a sequence set forth in SEQ ID NO: 137;
(viii) a mutant polypeptide comprising a sequence set forth in SEQ ID NO: 139;
(ix) a mutant polypeptide comprising a sequence set forth in SEQ ID NO: 145,
   at a level that is reduced compared to the level of binding of the compound that binds CD131 to a polypeptide comprising a sequence set forth in SEQ ID NO: 192.

In one example, the level of binding (e.g., as determined by K_{D}) of the CD131-binding protein to the mutant polypeptide is reduced by at least about 3 fold or 4 fold or fold or 10 fold. For example, the level of binding to the mutant polypeptide is reduced by at least about 20 fold or 50 fold or 100 fold.

In one example, the affinity (K_{D}) of the CD131-binding protein for the mutant polypeptide is about 4×10⁻⁶ or greater, e.g., 4.5×10⁻⁶ or 1×10⁵.

In one example, the compound that binds to CD131 binds to or cross-reacts with a polypeptide comprising a sequence set forth in any one of SEQ ID NOs: 117, 118, 120-123, 125-130, 132-136, 138 or 140-148.

In one example, the compound that binds to CD131 binds to a polypeptide comprising a sequence set forth in SEQ ID NO: 127 with a higher affinity than it does to a polypeptide comprising a sequence set forth in SEQ ID NO: 192.

In one example, the compound is a CD131-binding protein that binds to or cross reacts with one or more (or all) of the following mutant polypeptide(s):
(i) a mutant polypeptide comprising a sequence set forth in SEQ ID NO: 135;
(ii) a mutant polypeptide comprising a sequence set forth in SEQ ID NO: 136; and/or
(iii) a mutant polypeptide comprising a sequence set forth in SEQ ID NO: 138.

Methods for determining binding of a CD131-binding protein to a polypeptide will be apparent to the skilled artisan. For example, the polypeptide is immobilized on a solid or semi-solid surface and the CD131-binding protein is contacted to the immobilized polypeptide. Binding is then determined, e.g., by surface plasmon resonance.

In one example, the CD131-binding protein comprising an antigen binding site is a protein comprising one or more antibody variable regions.

In one example, a CD131-binding protein described herein comprises at least a heavy chain variable region (V_{H}) and a light chain variable region (V_{L}), wherein the V_{H} and V_{L} bind to form a Fv comprising an antigen binding domain. The skilled artisan will understand that the antigen binding domain comprises the binding site of the antibody.

In one example, the V_{H} and the V_{L} are in a single polypeptide chain. For example, the protein is:
(i) a single chain Fv fragment (scFv);
(ii) a dimeric scFv (di-scFv);
(iii) one of (i) or (ii) linked to a constant region of an antibody, Fc or a heavy chain constant domain (C_{H}) 2 and/or C_{H}3; or
(iv) one of (i) or (ii) linked to a protein that binds to an immune effector cell.

In one example, the V_{L} and V_{H} are in separate polypeptide chains.

For example, the protein is:
(i) a diabody;
(ii) a triabody;
(iii) a tetrabody;
(iv) a Fab;
(v) a F(ab′)₂;
(vi) a Fv;
(vii) one of (i) to (vi) linked to a constant region of an antibody, Fe or a heavy chain constant domain (C_{H}) 2 and/or C_{H}3;
(viii) one of (i) to (vi) linked to a protein that binds to an immune effector cell.

The foregoing proteins (described in the previous two lists) can also be referred to as antigen binding domains of antibodies.

In one example, the protein is an antibody, for example, a monoclonal antibody. In one example, the antibody is a naked antibody.

In one example, a protein (or antibody) is chimeric, de-immunized, humanized, human or primatized.

In one example, the protein or antibody is human. For example, the present disclosure provides an antibody which binds to or specifically binds to CD131 and neutralizes signaling by IL-3, IL-5 and GM-CSF, and wherein the antibody comprises an antigen binding domain or a V_{H} and/or V_{L} as described herein in any example.

Exemplary antibodies include 9A2-VR24.29 (also, referred to as “CSL311”) described in WO 2017/088028.

In one example, the compound is a CD131-binding protein comprising an antigen binding domain of an antibody, wherein the antigen binding domain binds to or specifically binds to CD131 and neutralizes signalling by IL-3, IL-5 and GM-CSF, and wherein the CD131-binding protein competitively inhibits binding of antibody 9A2 (comprising a V_{L} comprising a sequence set forth in SEQ ID NO: 5 and a V_{H} comprising a sequence set forth in SEQ ID NO: 20) to CD131.

In one example, the compound is a CD131-binding protein comprising an antigen binding domain of an antibody, wherein the antigen binding domain binds to or specifically binds to CD131 and neutralizes signalling by IL-3, IL-5 and GM-CSF, and wherein the CD131-binding protein competitively inhibits binding of antibody 9A2 (comprising a V_{L} comprising a sequence set forth in SEQ ID NO: 5 and a human kappa light chain constant region and a V_{H} comprising a sequence set forth in SEQ ID NO: 20 and a human IgG4 constant region) to CD131.

In one example, the compound is a CD131-binding protein comprising an antigen binding domain of an antibody, wherein the antigen binding domain binds to or specifically binds to CD131 and neutralizes signalling by IL-3, IL-5 and GM-CSF, and wherein the CD131-binding protein competitively inhibits binding of antibody 9A2 (comprising a light chain comprising a sequence set forth in SEQ ID NO: 5 and a heavy chain comprising a sequence set forth in SEQ ID NO: 20) to CD131.

In one example, the compound is a CD131-binding protein comprising an antigen binding domain of an antibody, wherein the antigen binding domain binds to a polypeptide comprising a sequence set forth in SEQ ID NO: 127 with a higher affinity than it does to a polypeptide comprising a sequence set forth in SEQ ID NO: 192.

In one example, the compound is a CD131-binding protein comprising an antigen binding domain of an antibody, wherein the antigen binding domain comprises a V_{H} and a V_{L}, wherein:
(A) (i) the V_{H} comprises a CDR1 comprising a sequence set forth between amino acids 26-35 of SEQ ID NO: 180, a CDR2 comprising a sequence set forth between amino acids 50-66 of SEQ ID NO: 180 and a CDR3 comprising a sequence set forth between amino acids 99-106 of SEQ ID NO: 180 or the V_{H} comprising a sequence set forth in SEQ ID NO: 180; and
   (ii) the V_{L} comprising a CDR1 comprising a sequence set forth between amino acids 24-34 of SEQ ID NO: 177, a CDR2 comprising a sequence set forth between amino acids 44-51 of SEQ ID NO: 177 and a CDR3 comprising a sequence set forth between amino acids 89-97 of SEQ ID NO: 177 or the V_{L} comprising a sequence set forth in SEQ ID NO: 177; or
(B) the V_{H} comprises a sequence set forth in SEQ ID NO: 193 and the V_{L} comprises a sequence set forth in SEQ ID NO: 5.

In one example, the compound is a CD131-binding protein comprising an antigen binding domain of an antibody, wherein the antigen binding domain comprises:
(i) a V_{H} comprising CDRs 1, 2 and 3 of a sequence set forth in SEQ ID NO: 20 and a V_{L} comprising CDRs 1, 2 and 3 of a sequence set forth in SEQ ID NO: 5;
(ii) a V_{H} comprising a sequence set forth in SEQ ID NO: 20 and a V_{L} comprising a sequence set forth in SEQ ID NO: 5;
(iii) a V_{H} comprising CDRs 1, 2 and 3 of a sequence set forth in SEQ ID NO: 20 and a V_{L} comprising CDRs 1, 2 and 3 of a sequence set forth in SEQ ID NO: 6;
(iv) a V_{H} comprising a sequence set forth in SEQ ID NO: 20 and a V_{L} comprising a sequence set forth in SEQ ID NO: 6;
(v) a V_{H} comprising CDRs 1, 2 and 3 of a sequence set forth in SEQ ID NO: 20 and a V_{L} comprising CDRs 1, 2 and 3 of a sequence set forth in SEQ ID NO: 7;
(vi) a V_{H} comprising a sequence set forth in SEQ ID NO: 20 and a V_{L} comprising a sequence set forth in SEQ ID NO: 7;
(vii) a V_{H} comprising CDRs 1, 2 and 3 of a sequence set forth in SEQ ID NO: 20 and a V_{L} comprising CDRs 1, 2 and 3 of a sequence set forth in SEQ ID NO: 8;
(viii) a V_{H} comprising a sequence set forth in SEQ ID NO: 20 and a V_{L} comprising a sequence set forth in SEQ ID NO: 8;
(ix) a V_{H} comprising CDRs 1, 2 and 3 of a sequence set forth in SEQ ID NO: 20 and a V_{L} comprising CDRs 1, 2 and 3 of a sequence set forth in SEQ ID NO: 9;
(x) a V_{H} comprising a sequence set forth in SEQ ID NO: 20 and a V_{L} comprising a sequence set forth in SEQ ID NO: 9;
(xi) a V_{H} comprising CDRs 1, 2 and 3 of a sequence set forth in SEQ ID NO: 20 and a V_{L} comprising CDRs 1, 2 and 3 of a sequence set forth in SEQ ID NO: 10;
(xii) a V_{H} comprising a sequence set forth in SEQ ID NO: 20 and a V_{L} comprising a sequence set forth in SEQ ID NO: 10;
(xiii) a V_{H} comprising CDRs 1, 2 and 3 of a sequence set forth in SEQ ID NO: 20 and a V_{L} comprising CDRs 1, 2 and 3 of a sequence set forth in SEQ ID NO: 11;
(xiv) a V_{H} comprising a sequence set forth in SEQ ID NO: 20 and a V_{L} comprising a sequence set forth in SEQ ID NO: 11;
(xv) a V_{H} comprising CDRs 1, 2 and 3 of a sequence set forth in SEQ ID NO: 20 and a V_{L} comprising CDRs 1, 2 and 3 of a sequence set forth in SEQ ID NO: 12;
(xvi) a V_{H} comprising a sequence set forth in SEQ ID NO: 20 and a V_{L} comprising a sequence set forth in SEQ ID NO: 12;
(xvii) a V_{H} comprising CDRs 1, 2 and 3 of a sequence set forth in SEQ ID NO: 20 and a V_{L} comprising CDRs 1, 2 and 3 of a sequence set forth in SEQ ID NO: 13;
(xviii) a V_{H} comprising a sequence set forth in SEQ ID NO: 20 and a V_{L} comprising a sequence set forth in SEQ ID NO: 13;
(xix) a V_{H} comprising CDRs 1, 2 and 3 of a sequence set forth in SEQ ID NO: 20 and a V_{L} comprising CDRs 1, 2 and 3 of a sequence set forth in SEQ ID NO: 14;
(xx) a V_{H} comprising a sequence set forth in SEQ ID NO: 20 and a V_{L} comprising a sequence set forth in SEQ ID NO: 14;
(xxi) a V_{H} comprising CDRs 1, 2 and 3 of a sequence set forth in SEQ ID NO: 20 and a V_{L} comprising CDRs 1, 2 and 3 of a sequence set forth in SEQ ID NO: 15;
(xxii) a V_{H} comprising a sequence set forth in SEQ ID NO: 20 and a V_{L} comprising a sequence set forth in SEQ ID NO: 15;
(xxiii) a V_{H} comprising CDRs 1, 2 and 3 of a sequence set forth in SEQ ID NO: 21 and a V_{L} comprising CDRs 1, 2 and 3 of a sequence set forth in SEQ ID NO: 5;
(xxiv) a V_{H} comprising a sequence set forth in SEQ ID NO: 21 and a V_{L} comprising a sequence set forth in SEQ ID NO: 5;
(xxv) a V_{H} comprising CDRs 1, 2 and 3 of a sequence set forth in SEQ ID NO: 22 and a V_{L} comprising CDRs 1, 2 and 3 of a sequence set forth in SEQ ID NO: 5;
(xxvi) a V_{H} comprising a sequence set forth in SEQ ID NO: 22 and a V_{L} comprising a sequence set forth in SEQ ID NO: 5;
(xxvii) a V_{H} comprising CDRs 1, 2 and 3 of a sequence set forth in SEQ ID NO: 23 and a V_{L} comprising CDRs 1, 2 and 3 of a sequence set forth in SEQ ID NO: 5;
(xxviii) a V_{H} comprising a sequence set forth in SEQ ID NO: 23 and a V_{L} comprising a sequence set forth in SEQ ID NO: 5;
(xxix) a V_{H} comprising CDRs 1, 2 and 3 of a sequence set forth in SEQ ID NO: 24 and a V_{L} comprising CDRs 1, 2 and 3 of a sequence set forth in SEQ ID NO: 5;
(xxx) a V_{H} comprising a sequence set forth in SEQ ID NO: 24 and a V_{L} comprising a sequence set forth in SEQ ID NO: 5;
(xxxi) a V_{H} comprising CDRs 1, 2 and 3 of a sequence set forth in SEQ ID NO: 25 and a V_{L} comprising CDRs 1, 2 and 3 of a sequence set forth in SEQ ID NO: 5;
(xxxii) a V_{H} comprising a sequence set forth in SEQ ID NO: 25 and a V_{L} comprising a sequence set forth in SEQ ID NO: 5;
(xxxiii) a V_{H} comprising CDRs 1, 2 and 3 of a sequence set forth in SEQ ID NO: 26 and a V_{L} comprising CDRs 1, 2 and 3 of a sequence set forth in SEQ ID NO: 5;
(xxxiv) a V_{H} comprising a sequence set forth in SEQ ID NO: 26 and a V_{L} comprising a sequence set forth in SEQ ID NO: 5;
(xxxv) a V_{H} comprising CDRs 1, 2 and 3 of a sequence set forth in SEQ ID NO: 27 and a V_{L} comprising CDRs 1, 2 and 3 of a sequence set forth in SEQ ID NO: 5;
(xxxvi) a V_{H} comprising a sequence set forth in SEQ ID NO: 27 and a V_{L} comprising a sequence set forth in SEQ ID NO: 5;
(xxxvii) a V_{H} comprising CDRs 1, 2 and 3 of a sequence set forth in SEQ ID NO: 28 and a V_{L} comprising CDRs 1, 2 and 3 of a sequence set forth in SEQ ID NO: 5;
(xxxviii) a V_{H} comprising a sequence set forth in SEQ ID NO: 28 and a V_{L} comprising a sequence set forth in SEQ ID NO: 5;
(xxxix) a V_{H} comprising CDRs 1, 2 and 3 of a sequence set forth in SEQ ID NO: 29 and a V_{L} comprising CDRs 1, 2 and 3 of a sequence set forth in SEQ ID NO: 5;
(xl) a V_{H} comprising a sequence set forth in SEQ ID NO: 29 and a V_{L} comprising a sequence set forth in SEQ ID NO: 5;
(xli) a V_{H} comprising CDRs 1, 2 and 3 of a sequence set forth in SEQ ID NO: 30 and a V_{L} comprising CDRs 1, 2 and 3 of a sequence set forth in SEQ ID NO: 5;
(xlii) a V_{H} comprising a sequence set forth in SEQ ID NO: 30 and a V_{L} comprising a sequence set forth in SEQ ID NO: 5;
(xliii) a V_{H} comprising CDRs 1, 2 and 3 of a sequence set forth in SEQ ID NO: 31 and a V_{L} comprising CDRs 1, 2 and 3 of a sequence set forth in SEQ ID NO: 5;
(xliv) a V_{H} comprising a sequence set forth in SEQ ID NO: 31 and a V_{L} comprising a sequence set forth in SEQ ID NO: 5;
(xlv) a V_{H} comprising CDRs 1, 2 and 3 of a sequence set forth in SEQ ID NO: 32 and a V_{L} comprising CDRs 1, 2 and 3 of a sequence set forth in SEQ ID NO: 5;
(xlvi) a V_{H} comprising a sequence set forth in SEQ ID NO: 32 and a V_{L} comprising a sequence set forth in SEQ ID NO: 5;
(xlvii) a V_{H} comprising CDRs 1, 2 and 3 of a sequence set forth in SEQ ID NO: 33 and a V_{L} comprising CDRs 1, 2 and 3 of a sequence set forth in SEQ ID NO: 5;
(xlviii) a V_{H} comprising a sequence set forth in SEQ ID NO: 33 and a V_{L} comprising a sequence set forth in SEQ ID NO: 5;
(xlix) a V_{H} comprising CDRs 1, 2 and 3 of a sequence set forth in SEQ ID NO: 34 and a V_{L} comprising CDRs 1, 2 and 3 of a sequence set forth in SEQ ID NO: 5;
(1) a V_{H} comprising a sequence set forth in SEQ ID NO: 34 and a V_{L} comprising a sequence set forth in SEQ ID NO: 5;
(li) a V_{H} comprising CDRs 1, 2 and 3 of a sequence set forth in SEQ ID NO: 35 and a V_{L} comprising CDRs 1, 2 and 3 of a sequence set forth in SEQ ID NO: 5;
(lii) a V_{H} comprising a sequence set forth in SEQ ID NO: 35 and a V_{L} comprising a sequence set forth in SEQ ID NO: 5;
(liii) a V_{H} comprising CDRs 1, 2 and 3 of a sequence set forth in SEQ ID NO: 36 and a V_{L} comprising CDRs 1, 2 and 3 of a sequence set forth in SEQ ID NO: 5;
(liv) a V_{H} comprising a sequence set forth in SEQ ID NO: 36 and a V_{L} comprising a sequence set forth in SEQ ID NO: 5;
(lv) a V_{H} comprising CDRs 1, 2 and 3 of a sequence set forth in SEQ ID NO: 37 and a V_{L} comprising CDRs 1, 2 and 3 of a sequence set forth in SEQ ID NO: 5;
(lvi) a V_{H} comprising a sequence set forth in SEQ ID NO: 37 and a V_{L} comprising a sequence set forth in SEQ ID NO: 5;
(lvii) a V_{H} comprising CDRs 1, 2 and 3 of a sequence set forth in SEQ ID NO: 38 and a V_{L} comprising CDRs 1, 2 and 3 of a sequence set forth in SEQ ID NO: 5;
(lviii) a V_{H} comprising a sequence set forth in SEQ ID NO: 38 and a V_{L} comprising a sequence set forth in SEQ ID NO: 5;
(lix) a V_{H} comprising CDRs 1, 2 and 3 of a sequence set forth in SEQ ID NO: 39 and a V_{L} comprising CDRs 1, 2 and 3 of a sequence set forth in SEQ ID NO: 5;
(lx) a V_{H} comprising a sequence set forth in SEQ ID NO: 39 and a V_{L} comprising a sequence set forth in SEQ ID NO: 5;
(lxi) a V_{H} comprising CDRs 1, 2 and 3 of a sequence set forth in SEQ ID NO: 40 and a V_{L} comprising CDRs 1, 2 and 3 of a sequence set forth in SEQ ID NO: 5;
(lxii) a V_{H} comprising a sequence set forth in SEQ ID NO: 40 and a V_{L} comprising a sequence set forth in SEQ ID NO: 5;
(lxiii) a V_{H} comprising CDRs 1, 2 and 3 of a sequence set forth in SEQ ID NO: 41 and a V_{L} comprising CDRs 1, 2 and 3 of a sequence set forth in SEQ ID NO: 5;
(lxiv) a V_{H} comprising a sequence set forth in SEQ ID NO: 41 and a V_{L} comprising a sequence set forth in SEQ ID NO: 5;
(lxv) a V_{H} comprising CDRs 1, 2 and 3 of a sequence set forth in SEQ ID NO: 42 and a V_{L} comprising CDRs 1, 2 and 3 of a sequence set forth in SEQ ID NO: 5;
(lxvi) a V_{H} comprising a sequence set forth in SEQ ID NO: 42 and a V_{L} comprising a sequence set forth in SEQ ID NO: 5;
(lxvii) a V_{H} comprising CDRs 1, 2 and 3 of a sequence set forth in SEQ ID NO: 43 and a V_{L} comprising CDRs 1, 2 and 3 of a sequence set forth in SEQ ID NO: 5;
(lxviii) a V_{H} comprising a sequence set forth in SEQ ID NO: 43 and a V_{L} comprising a sequence set forth in SEQ ID NO: 5;
(lxix) a V_{H} comprising CDRs 1, 2 and 3 of a sequence set forth in SEQ ID NO: 44 and a V_{L} comprising CDRs 1, 2 and 3 of a sequence set forth in SEQ ID NO: 5;
(lxx) a V_{H} comprising a sequence set forth in SEQ ID NO: 44 and a V_{L} comprising a sequence set forth in SEQ ID NO: 5;
(lxxi) a V_{H} comprising CDRs 1, 2 and 3 of a sequence set forth in SEQ ID NO: 45 and a V_{L} comprising CDRs 1, 2 and 3 of a sequence set forth in SEQ ID NO: 5;
(lxxii) a V_{H} comprising a sequence set forth in SEQ ID NO: 45 and a V_{L} comprising a sequence set forth in SEQ ID NO: 5;
(lxxiii) a V_{H} comprising CDRs 1, 2 and 3 of a sequence set forth in SEQ ID NO: 46 and a V_{L} comprising CDRs 1, 2 and 3 of a sequence set forth in SEQ ID NO: 5;
(lxxiv) a V_{H} comprising a sequence set forth in SEQ ID NO: 46 and a V_{L} comprising a sequence set forth in SEQ ID NO: 5;
(lxxv) a V_{H} comprising CDRs 1, 2 and 3 of a sequence set forth in SEQ ID NO: 47 and a V_{L} comprising CDRs 1, 2 and 3 of a sequence set forth in SEQ ID NO: 5;
(lxxvi) a V_{H} comprising a sequence set forth in SEQ ID NO: 47 and a V_{L} comprising a sequence set forth in SEQ ID NO: 5;
(lxxvii) a V_{H} comprising CDRs 1, 2 and 3 of a sequence set forth in SEQ ID NO: 48 and a V_{L} comprising CDRs 1, 2 and 3 of a sequence set forth in SEQ ID NO: 5;
(lxxviii) a V_{H} comprising a sequence set forth in SEQ ID NO: 48 and a V_{L} comprising a sequence set forth in SEQ ID NO: 5;
(lxxix) a V_{H} comprising CDRs 1, 2 and 3 of a sequence set forth in SEQ ID NO: 49 and a V_{L} comprising CDRs 1, 2 and 3 of a sequence set forth in SEQ ID NO: 5;
(lxxx) a V_{H} comprising a sequence set forth in SEQ ID NO: 49 and a V_{L} comprising a sequence set forth in SEQ ID NO: 5;
(lxxxi) a V_{H} comprising CDRs 1, 2 and 3 of a sequence set forth in SEQ ID NO: 50 and a V_{L} comprising CDRs 1, 2 and 3 of a sequence set forth in SEQ ID NO: 5;
(lxxxii) a V_{H} comprising a sequence set forth in SEQ ID NO: 50 and a V_{L} comprising a sequence set forth in SEQ ID NO: 5;
(lxxxiii) a V_{H} comprising CDRs 1, 2 and 3 of a sequence set forth in SEQ ID NO: 51 and a V_{L} comprising CDRs 1, 2 and 3 of a sequence set forth in SEQ ID NO: 5;
(lxxxiv) a V_{H} comprising a sequence set forth in SEQ ID NO: 51 and a V_{L} comprising a sequence set forth in SEQ ID NO: 5;
(lxxxv) a V_{H} comprising CDRs 1, 2 and 3 of a sequence set forth in SEQ ID NO: 52 and a V_{L} comprising CDRs 1, 2 and 3 of a sequence set forth in SEQ ID NO: 5;
(lxxxvi) a V_{H} comprising a sequence set forth in SEQ ID NO: 52 and a V_{L} comprising a sequence set forth in SEQ ID NO: 5;
(lxxxvii) a V_{H} comprising CDRs 1, 2 and 3 of a sequence set forth in SEQ ID NO: 53 and a V_{L} comprising CDRs 1, 2 and 3 of a sequence set forth in SEQ ID NO: 5;
(lxxxviii) a V_{H} comprising a sequence set forth in SEQ ID NO: 53 and a V_{L} comprising a sequence set forth in SEQ ID NO: 5;
(lxxxix) a V_{H} comprising CDRs 1, 2 and 3 of a sequence set forth in SEQ ID NO: 54 and a V_{L} comprising CDRs 1, 2 and 3 of a sequence set forth in SEQ ID NO: 5;
(xc) a V_{H} comprising a sequence set forth in SEQ ID NO: 54 and a V_{L} comprising a sequence set forth in SEQ ID NO: 5;
(xci) a V_{H} comprising CDRs 1, 2 and 3 of a sequence set forth in SEQ ID NO: 55 and a V_{L} comprising CDRs 1, 2 and 3 of a sequence set forth in SEQ ID NO: 5;
(xcii) a V_{H} comprising a sequence set forth in SEQ ID NO: 55 and a V_{L} comprising a sequence set forth in SEQ ID NO: 5;
(xciii) a V_{H} comprising CDRs 1, 2 and 3 of a sequence set forth in SEQ ID NO: 56 and a V_{L} comprising CDRs 1, 2 and 3 of a sequence set forth in SEQ ID NO: 5;
(xciv) a V_{H} comprising a sequence set forth in SEQ ID NO: 56 and a V_{L} comprising a sequence set forth in SEQ ID NO: 5;
(xcv) a V_{H} comprising CDRs 1, 2 and 3 of a sequence set forth in SEQ ID NO: 57 and a V_{L} comprising CDRs 1, 2 and 3 of a sequence set forth in SEQ ID NO: 5;
(xcvi) a V_{H} comprising a sequence set forth in SEQ ID NO: 57 and a V_{L} comprising a sequence set forth in SEQ ID NO: 5;
(xcvii) a V_{H} comprising CDRs 1, 2 and 3 of a sequence set forth in SEQ ID NO: 58 and a V_{L} comprising CDRs 1, 2 and 3 of a sequence set forth in SEQ ID NO: 5;
(xcviii) a V_{H} comprising a sequence set forth in SEQ ID NO: 58 and a V_{L} comprising a sequence set forth in SEQ ID NO: 5;
(xcix) a V_{H} comprising CDRs 1, 2 and 3 of a sequence set forth in SEQ ID NO: 59 and a V_{L} comprising CDRs 1, 2 and 3 of a sequence set forth in SEQ ID NO: 5;
(c) a V_{H} comprising a sequence set forth in SEQ ID NO: 59 and a V_{L} comprising a sequence set forth in SEQ ID NO: 5;
(ci) a V_{H} comprising CDRs 1, 2 and 3 of a sequence set forth in SEQ ID NO: 60 and a V_{L} comprising CDRs 1, 2 and 3 of a sequence set forth in SEQ ID NO: 5;
(cii) a V_{H} comprising a sequence set forth in SEQ ID NO: 60 and a V_{L} comprising a sequence set forth in SEQ ID NO: 5;
(ciii) a V_{H} comprising CDRs 1, 2 and 3 of a sequence set forth in SEQ ID NO: 61 and a V_{L} comprising CDRs 1, 2 and 3 of a sequence set forth in SEQ ID NO: 5;
(civ) a V_{H} comprising a sequence set forth in SEQ ID NO: 61 and a V_{L} comprising a sequence set forth in SEQ ID NO: 5;
(cv) a V_{H} comprising CDRs 1, 2 and 3 of a sequence set forth in SEQ ID NO: 62 and a V_{L} comprising CDRs 1, 2 and 3 of a sequence set forth in SEQ ID NO: 5;
(cvi) a V_{H} comprising a sequence set forth in SEQ ID NO: 62 and a V_{L} comprising a sequence set forth in SEQ ID NO: 5;
(cvii) a V_{H} comprising CDRs 1, 2 and 3 of a sequence set forth in SEQ ID NO: 63 and a V_{L} comprising CDRs 1, 2 and 3 of a sequence set forth in SEQ ID NO: 5;
(cviii) a V_{H} comprising a sequence set forth in SEQ ID NO: 63 and a V_{L} comprising a sequence set forth in SEQ ID NO: 5;
(cix) a V_{H} comprising CDRs 1, 2 and 3 of a sequence set forth in SEQ ID NO: 64 and a V_{L} comprising CDRs 1, 2 and 3 of a sequence set forth in SEQ ID NO: 5;
(cx) a V_{H} comprising a sequence set forth in SEQ ID NO: 64 and a V_{L} comprising a sequence set forth in SEQ ID NO: 5;
(cxi) a V_{H} comprising CDRs 1, 2 and 3 of a sequence set forth in SEQ ID NO: 65 and a V_{L} comprising CDRs 1, 2 and 3 of a sequence set forth in SEQ ID NO: 5;
(cxii) a V_{H} comprising a sequence set forth in SEQ ID NO: 65 and a V_{L} comprising a sequence set forth in SEQ ID NO: 5;
(cxiii) a V_{H} comprising CDRs 1, 2 and 3 of a sequence set forth in SEQ ID NO: 66 and a V_{L} comprising CDRs 1, 2 and 3 of a sequence set forth in SEQ ID NO: 5;
(cxiv) a V_{H} comprising a sequence set forth in SEQ ID NO: 66 and a V_{L} comprising a sequence set forth in SEQ ID NO: 5;
(cxv) a V_{H} comprising CDRs 1, 2 and 3 of a sequence set forth in SEQ ID NO: 67 and a V_{L} comprising CDRs 1, 2 and 3 of a sequence set forth in SEQ ID NO: 5;
(cxvi) a V_{H} comprising a sequence set forth in SEQ ID NO: 67 and a V_{L} comprising a sequence set forth in SEQ ID NO: 5;
(cxvii) a V_{H} comprising CDRs 1, 2 and 3 of a sequence set forth in SEQ ID NO: 68 and a V_{L} comprising CDRs 1, 2 and 3 of a sequence set forth in SEQ ID NO: 5;
(cxviii) a V_{H} comprising a sequence set forth in SEQ ID NO: 68 and a V_{L} comprising a sequence set forth in SEQ ID NO: 5;
(cxix) a V_{H} comprising CDRs 1, 2 and 3 of a sequence set forth in SEQ ID NO: 69 and a V_{L} comprising CDRs 1, 2 and 3 of a sequence set forth in SEQ ID NO: 5;
(cxx) a V_{H} comprising a sequence set forth in SEQ ID NO: 69 and a V_{L} comprising a sequence set forth in SEQ ID NO: 5;
(cxxi) a V_{H} comprising CDRs 1, 2 and 3 of a sequence set forth in SEQ ID NO: 70 and a V_{L} comprising CDRs 1, 2 and 3 of a sequence set forth in SEQ ID NO: 5;
(cxxii) a V_{H} comprising a sequence set forth in SEQ ID NO: 70 and a V_{L} comprising a sequence set forth in SEQ ID NO: 5;
(cxxiii) a V_{H} comprising CDRs 1, 2 and 3 of a sequence set forth in SEQ ID NO: 71 and a V_{L} comprising CDRs 1, 2 and 3 of a sequence set forth in SEQ ID NO: 5;
(cxxiv) a V_{H} comprising a sequence set forth in SEQ ID NO: 71 and a V_{L} comprising a sequence set forth in SEQ ID NO: 5;
(cxxv) a V_{H} comprising CDRs 1, 2 and 3 of a sequence set forth in SEQ ID NO: 72 and a V_{L} comprising CDRs 1, 2 and 3 of a sequence set forth in SEQ ID NO: 5;
(cxxvi) a V_{H} comprising a sequence set forth in SEQ ID NO: 72 and a V_{L} comprising a sequence set forth in SEQ ID NO: 5;
(cxxvii) a V_{H} comprising CDRs 1, 2 and 3 of a sequence set forth in SEQ ID NO: 73 and a V_{L} comprising CDRs 1, 2 and 3 of a sequence set forth in SEQ ID NO: 5;
(cxxviii) a V_{H} comprising a sequence set forth in SEQ ID NO: 73 and a V_{L} comprising a sequence set forth in SEQ ID NO: 5;
(cxxix) a V_{H} comprising CDRs 1, 2 and 3 of a sequence set forth in SEQ ID NO: 75 and a V_{L} comprising CDRs 1, 2 and 3 of a sequence set forth in SEQ ID NO: 5;
(cxxx) a V_{H} comprising a sequence set forth in SEQ ID NO: 75 and a V_{L} comprising a sequence set forth in SEQ ID NO: 5;
(cxxxi) a V_{H} comprising CDRs 1, 2 and 3 of a sequence set forth in SEQ ID NO: 76 and a V_{L} comprising CDRs 1, 2 and 3 of a sequence set forth in SEQ ID NO: 5;
(cxxxii) a V_{H} comprising a sequence set forth in SEQ ID NO: 76 and a V_{L} comprising a sequence set forth in SEQ ID NO: 5;
(cxxxiii) a V_{H} comprising CDRs 1, 2 and 3 of a sequence set forth in SEQ ID NO: 77 and a V_{L} comprising CDRs 1, 2 and 3 of a sequence set forth in SEQ ID NO: 5;
(cxxxiv) a V_{H} comprising a sequence set forth in SEQ ID NO: 77 and a V_{L} comprising a sequence set forth in SEQ ID NO: 5;
(cxxxv) a V_{H} comprising CDRs 1, 2 and 3 of a sequence set forth in SEQ ID NO: 78 and a V_{L} comprising CDRs 1, 2 and 3 of a sequence set forth in SEQ ID NO: 5;
(cxxxvi) a V_{H} comprising a sequence set forth in SEQ ID NO: 78 and a V_{L} comprising a sequence set forth in SEQ ID NO: 5;
(cxxxvii) a V_{H} comprising CDRs 1, 2 and 3 of a sequence set forth in SEQ ID NO: 79 and a V_{L} comprising CDRs 1, 2 and 3 of a sequence set forth in SEQ ID NO: 5;
(cxxxviii) a V_{H} comprising a sequence set forth in SEQ ID NO: 79 and a V_{L} comprising a sequence set forth in SEQ ID NO: 5;
(cxxxix) a V_{H} comprising CDRs 1, 2 and 3 of a sequence set forth in SEQ ID NO: 80 and a V_{L} comprising CDRs 1, 2 and 3 of a sequence set forth in SEQ ID NO: 5;
(cxl) a V_{H} comprising a sequence set forth in SEQ ID NO: 80 and a V_{L} comprising a sequence set forth in SEQ ID NO: 5;
(cxli) a V_{H} comprising CDRs 1, 2 and 3 of a sequence set forth in SEQ ID NO: 81 and a V_{L} comprising CDRs 1, 2 and 3 of a sequence set forth in SEQ ID NO: 5;
(cxlii) a V_{H} comprising a sequence set forth in SEQ ID NO: 81 and a V_{L} comprising a sequence set forth in SEQ ID NO: 5;
(cxliii) a V_{H} comprising CDRs 1, 2 and 3 of a sequence set forth in SEQ ID NO: 82 and a V_{L} comprising CDRs 1, 2 and 3 of a sequence set forth in SEQ ID NO: 5;
(cxliv) a V_{H} comprising a sequence set forth in SEQ ID NO: 82 and a V_{L} comprising a sequence set forth in SEQ ID NO: 5;
(cxlv) a V_{H} comprising CDRs 1, 2 and 3 of a sequence set forth in SEQ ID NO: 83 and a V_{L} comprising CDRs 1, 2 and 3 of a sequence set forth in SEQ ID NO: 5;
(cxlvi) a V_{H} comprising a sequence set forth in SEQ ID NO: 83 and a V_{L} comprising a sequence set forth in SEQ ID NO: 5;
(cxlvii) a V_{H} comprising CDRs 1, 2 and 3 of a sequence set forth in SEQ ID NO: 84 and a V_{L} comprising CDRs 1, 2 and 3 of a sequence set forth in SEQ ID NO: 5;
(cxlviii) a V_{H} comprising a sequence set forth in SEQ ID NO: 84 and a V_{L} comprising a sequence set forth in SEQ ID NO: 5;
(cxlix) a V_{H} comprising CDRs 1, 2 and 3 of a sequence set forth in SEQ ID NO: 85 and a V_{L} comprising CDRs 1, 2 and 3 of a sequence set forth in SEQ ID NO: 5;
(cl) a V_{H} comprising a sequence set forth in SEQ ID NO: 85 and a V_{L} comprising a sequence set forth in SEQ ID NO: 5;
(cli) a V_{H} comprising CDRs 1, 2 and 3 of a sequence set forth in SEQ ID NO: 86 and a V_{L} comprising CDRs 1, 2 and 3 of a sequence set forth in SEQ ID NO: 5;
(clii) a V_{H} comprising a sequence set forth in SEQ ID NO: 86 and a V_{L} comprising a sequence set forth in SEQ ID NO: 5;
(cliii) a V_{H} comprising CDRs 1, 2 and 3 of a sequence set forth in SEQ ID NO: 87 and a V_{L} comprising CDRs 1, 2 and 3 of a sequence set forth in SEQ ID NO: 5;
(cliv) a V_{H} comprising a sequence set forth in SEQ ID NO: 87 and a V_{L} comprising a sequence set forth in SEQ ID NO: 5;
(clv) a V_{H} comprising CDRs 1, 2 and 3 of a sequence set forth in SEQ ID NO: 88 and a V_{L} comprising CDRs 1, 2 and 3 of a sequence set forth in SEQ ID NO: 5;
(clvi) a V_{H} comprising a sequence set forth in SEQ ID NO: 88 and a V_{L} comprising a sequence set forth in SEQ ID NO: 5;
(clvii) a V_{H} comprising CDRs 1, 2 and 3 of a sequence set forth in SEQ ID NO: 89 and a V_{L} comprising CDRs 1, 2 and 3 of a sequence set forth in SEQ ID NO: 5;
(clviii) a V_{H} comprising a sequence set forth in SEQ ID NO: 89 and a V_{L} comprising a sequence set forth in SEQ ID NO: 5;
(clix) a V_{H} comprising CDRs 1, 2 and 3 of a sequence set forth in SEQ ID NO: 90 and a V_{L} comprising CDRs 1, 2 and 3 of a sequence set forth in SEQ ID NO: 5;
(clx) a V_{H} comprising a sequence set forth in SEQ ID NO: 90 and a V_{L} comprising a sequence set forth in SEQ ID NO: 5;
(clxi) a V_{H} comprising CDRs 1, 2 and 3 of a sequence set forth in SEQ ID NO: 91 and a V_{L} comprising CDRs 1, 2 and 3 of a sequence set forth in SEQ ID NO: 5;
(clxii) a V_{H} comprising a sequence set forth in SEQ ID NO: 91 and a V_{L} comprising a sequence set forth in SEQ ID NO: 5;
(clxiii) a V_{H} comprising CDRs 1, 2 and 3 of a sequence set forth in SEQ ID NO: 92 and a V_{L} comprising CDRs 1, 2 and 3 of a sequence set forth in SEQ ID NO: 5;
(clxiv) a V_{H} comprising a sequence set forth in SEQ ID NO: 92 and a V_{L} comprising a sequence set forth in SEQ ID NO: 5;
(clxv) a V_{H} comprising CDRs 1, 2 and 3 of a sequence set forth in SEQ ID NO: 93 and a V_{L} comprising CDRs 1, 2 and 3 of a sequence set forth in SEQ ID NO: 5;
(clxvi) a V_{H} comprising a sequence set forth in SEQ ID NO: 93 and a V_{L} comprising a sequence set forth in SEQ ID NO: 5;
(clxvii) a V_{H} comprising CDRs 1, 2 and 3 of a sequence set forth in SEQ ID NO: 94 and a V_{L} comprising CDRs 1, 2 and 3 of a sequence set forth in SEQ ID NO: 5;
(clxviii) a V_{H} comprising a sequence set forth in SEQ ID NO: 94 and a V_{L} comprising a sequence set forth in SEQ ID NO: 5;
(clxix) a V_{H} comprising CDRs 1, 2 and 3 of a sequence set forth in SEQ ID NO: 95 and a V_{L} comprising CDRs 1, 2 and 3 of a sequence set forth in SEQ ID NO: 5;
(clxx) a V_{H} comprising a sequence set forth in SEQ ID NO: 95 and a V_{L} comprising a sequence set forth in SEQ ID NO: 5;
(clxxi) a V_{H} comprising CDRs 1, 2 and 3 of a sequence set forth in SEQ ID NO: 96 and a V_{L} comprising CDRs 1, 2 and 3 of a sequence set forth in SEQ ID NO: 5;
(clxxii) a V_{H} comprising a sequence set forth in SEQ ID NO: 96 and a V_{L} comprising a sequence set forth in SEQ ID NO: 5;
(clxxiii) a V_{H} comprising CDRs 1, 2 and 3 of a sequence set forth in SEQ ID NO: 97 and a V_{L} comprising CDRs 1, 2 and 3 of a sequence set forth in SEQ ID NO: 5;
(clxxiv) a V_{H} comprising a sequence set forth in SEQ ID NO: 97 and a V_{L} comprising a sequence set forth in SEQ ID NO: 5;
(clxxv) a V_{H} comprising CDRs 1, 2 and 3 of a sequence set forth in SEQ ID NO: 98 and a V_{L} comprising CDRs 1, 2 and 3 of a sequence set forth in SEQ ID NO: 5;
(clxxvi) a V_{H} comprising a sequence set forth in SEQ ID NO: 98 and a V_{L} comprising a sequence set forth in SEQ ID NO: 5;
(clxxvii) a V_{H} comprising CDRs 1, 2 and 3 of a sequence set forth in SEQ ID NO: 99 and a V_{L} comprising CDRs 1, 2 and 3 of a sequence set forth in SEQ ID NO: 5;
(clxxviii) a V_{H} comprising a sequence set forth in SEQ ID NO: 99 and a V_{L} comprising a sequence set forth in SEQ ID NO: 5;
(clxxix) a V_{H} comprising CDRs 1, 2 and 3 of a sequence set forth in SEQ ID NO: 100 and a V_{L} comprising CDRs 1, 2 and 3 of a sequence set forth in SEQ ID NO: 5;
(clxxx) a V_{H} comprising a sequence set forth in SEQ ID NO: 100 and a V_{L} comprising a sequence set forth in SEQ ID NO: 5;
(clxxxi) a V_{H} comprising CDRs 1, 2 and 3 of a sequence set forth in SEQ ID NO: 101 and a V_{L} comprising CDRs 1, 2 and 3 of a sequence set forth in SEQ ID NO: 5;
(clxxxii) a V_{H} comprising a sequence set forth in SEQ ID NO: 101 and a V_{L} comprising a sequence set forth in SEQ ID NO: 5;
(clxxxiii) a V_{H} comprising CDRs 1, 2 and 3 of a sequence set forth in SEQ ID NO: 102 and a V_{L} comprising CDRs 1, 2 and 3 of a sequence set forth in SEQ ID NO: 5;
(clxxxiv) a V_{H} comprising a sequence set forth in SEQ ID NO: 102 and a V_{L} comprising a sequence set forth in SEQ ID NO: 5;
(clxxxv) a V_{H} comprising CDRs 1, 2 and 3 of a sequence set forth in SEQ ID NO: 103 and a V_{L} comprising CDRs 1, 2 and 3 of a sequence set forth in SEQ ID NO: 5;
(clxxxvi) a V_{H} comprising a sequence set forth in SEQ ID NO: 103 and a V_{L} comprising a sequence set forth in SEQ ID NO: 5;
(clxxxvii) a V_{H} comprising CDRs 1, 2 and 3 of a sequence set forth in SEQ ID NO: 104 and a V_{L} comprising CDRs 1, 2 and 3 of a sequence set forth in SEQ ID NO: 5;
(clxxxviii) a V_{H} comprising a sequence set forth in SEQ ID NO: 104 and a V_{L} comprising a sequence set forth in SEQ ID NO: 5;
(clxxxix) a V_{H} comprising CDRs 1, 2 and 3 of a sequence set forth in SEQ ID NO: 105 and a V_{L} comprising CDRs 1, 2 and 3 of a sequence set forth in SEQ ID NO: 5;
(cxc) a V_{H} comprising a sequence set forth in SEQ ID NO: 105 and a V_{L} comprising a sequence set forth in SEQ ID NO: 5;
(cxci) a V_{H} comprising CDRs 1, 2 and 3 of a sequence set forth in SEQ ID NO: 106 and a V_{L} comprising CDRs 1, 2 and 3 of a sequence set forth in SEQ ID NO: 5;
(cxcii) a V_{H} comprising a sequence set forth in SEQ ID NO: 106 and a V_{L} comprising a sequence set forth in SEQ ID NO: 5;
(cxciii) a V_{H} comprising CDRs 1, 2 and 3 of a sequence set forth in SEQ ID NO: 107 and a V_{L} comprising CDRs 1, 2 and 3 of a sequence set forth in SEQ ID NO: 5;
(cxciv) a V_{H} comprising a sequence set forth in SEQ ID NO: 107 and a V_{L} comprising a sequence set forth in SEQ ID NO: 5;
(cxcv) a V_{H} comprising CDRs 1, 2 and 3 of a sequence set forth in SEQ ID NO: 108 and a V_{L} comprising CDRs 1, 2 and 3 of a sequence set forth in SEQ ID NO: 5;
(cxcvi) a V_{H} comprising a sequence set forth in SEQ ID NO: 108 and a V_{L} comprising a sequence set forth in SEQ ID NO: 5;
(cxcvii) a V_{H} comprising CDRs 1, 2 and 3 of a sequence set forth in SEQ ID NO: 109 and a V_{L} comprising CDRs 1, 2 and 3 of a sequence set forth in SEQ ID NO: 5;
(cxcviii) a V_{H} comprising a sequence set forth in SEQ ID NO: 109 and a V_{L} comprising a sequence set forth in SEQ ID NO: 5;
(cxcix) a V_{H} comprising CDRs 1, 2 and 3 of a sequence set forth in SEQ ID NO: 110 and a V_{L} comprising CDRs 1, 2 and 3 of a sequence set forth in SEQ ID NO: 5;
(cc) a V_{H} comprising a sequence set forth in SEQ ID NO: 110 and a V_{L} comprising a sequence set forth in SEQ ID NO: 5;
(cci) a V_{H} comprising CDRs 1, 2 and 3 of a sequence set forth in SEQ ID NO: 111 and a V_{L} comprising CDRs 1, 2 and 3 of a sequence set forth in SEQ ID NO: 5;
(ccii) a V_{H} comprising a sequence set forth in SEQ ID NO: 111 and a V_{L} comprising a sequence set forth in SEQ ID NO: 5;
(cciii) a V_{H} comprising CDRs 1, 2 and 3 of a sequence set forth in SEQ ID NO: 112 and a V_{L} comprising CDRs 1, 2 and 3 of a sequence set forth in SEQ ID NO: 5;
(cciv) a V_{H} comprising a sequence set forth in SEQ ID NO: 112 and a V_{L} comprising a sequence set forth in SEQ ID NO: 5;
(ccv) a V_{H} comprising CDRs 1, 2 and 3 of a sequence set forth in SEQ ID NO: 113 and a V_{L} comprising CDRs 1, 2 and 3 of a sequence set forth in SEQ ID NO: 5;
(ccvi) a V_{H} comprising a sequence set forth in SEQ ID NO: 113 and a V_{L} comprising a sequence set forth in SEQ ID NO: 5;
(ccvii) a V_{H} comprising CDRs 1, 2 and 3 of a sequence set forth in SEQ ID NO: 114 and a V_{L} comprising CDRs 1, 2 and 3 of a sequence set forth in SEQ ID NO: 5;
(ccviii) a V_{H} comprising a sequence set forth in SEQ ID NO: 114 and a V_{L} comprising a sequence set forth in SEQ ID NO: 5;
(ccix) a V_{H} comprising CDRs 1, 2 and 3 of a sequence set forth in SEQ ID NO: 115 and a V_{L} comprising CDRs 1, 2 and 3 of a sequence set forth in SEQ ID NO: 5;
(ccx) a V_{H} comprising a sequence set forth in SEQ ID NO: 115 and a V_{L} comprising a sequence set forth in SEQ ID NO: 5;
(ccxi) a V_{H} comprising CDRs 1, 2 and 3 of a sequence set forth in SEQ ID NO: 116 and a V_{L} comprising CDRs 1, 2 and 3 of a sequence set forth in SEQ ID NO: 5; or
(ccxii) a V_{H} comprising a sequence set forth in SEQ ID NO: 116 and a V_{L} comprising a sequence set forth in SEQ ID NO: 5.

In one example, the compound is an antibody comprising:
(i) a V_{H} comprising CDRs 1, 2 and 3 of a sequence set forth in SEQ ID NO: 64 and a V_{L} comprising CDRs 1, 2 and 3 of a sequence set forth in SEQ ID NO: 5; or
(ii) a V_{H} comprising a sequence set forth in SEQ ID NO: 64 and a V_{L} comprising a sequence set forth in SEQ ID NO: 5.

In one example, the compound is an antibody comprising a V_{H} comprising CDRs 1, 2 and 3 of a sequence set forth in SEQ ID NO: 64 and a V_{L} comprising CDRs 1, 2 and 3 of a sequence set forth in SEQ ID NO: 5.

In one example, the compound is an antibody comprising a V_{H} comprising a sequence set forth in SEQ ID NO: 64 and a V_{L} comprising a sequence set forth in SEQ ID NO: 5.

In one example, the CD131-binding protein or antibody as described herein comprises a human constant region, e.g., an IgG constant region, such as an IgG1, IgG2, IgG3 or IgG4 constant region or mixtures thereof. In the case of an antibody or protein comprising a V_{H} and a V_{L}, the V_{H} can be linked to a heavy chain constant region and the V_{L} can be linked to a light chain constant region.

The C-terminal lysine of the heavy chain constant region of a whole antibody (or a CD131-binding protein comprising a constant region or a C_{H}3) of the disclosure may be removed, for example, during production or purification of the antibody or protein, or by recombinantly engineering the nucleic acid encoding a heavy chain of the antibody. Accordingly, whole antibodies (or CD131-binding proteins) may comprise populations with all C-terminal lysine residues removed, populations with no C-terminal lysine residues removed, and/or populations having a mixture of protein with and without the C-terminal lysine residue. In some examples, the populations may additionally comprise protein in which the C-terminal lysine residue is removed in one of the heavy chain constant regions. Similarly, a composition of whole antibodies may comprise the same or a similar mix of antibody populations with or without the C-terminal lysine residue.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, the heavy chain constant region comprises a sequence set forth in SEQ ID NO: 197. In one example a protein or antibody as described herein or a composition of a protein or antibody as described herein, comprises a heavy chain constant region, including a stabilized heavy chain constant region, comprising a mixture of sequences fully or partially with or without the C-terminal lysine residue.

In one example, an antibody of the disclosure comprises a V_{H} disclosed herein linked or fused to an IgG4 constant region or stabilized IgG4 constant region (e.g., as discussed above) and the V_{L} is linked to or fused to a kappa light chain constant region.

The functional characteristics of a compound that binds CD131 (i.e., a CD131-binding protein) of the disclosure will be taken to apply mutatis mutandis to an antibody of the disclosure.

In one example, the compound that binds to CD131 is within a composition. For example, the composition comprises a compound (e.g., CD131-binding protein or antibody) as described herein. In one example, the composition additionally comprises one or more variants of the compound (i.e., protein or antibody). For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, a method described herein comprises administering between about 0.05 mg/kg and 30 mg/kg of the compound that binds to CD131 or composition comprising the compound. For example, the method comprises administering between 0.1 mg/kg and 10 mg/kg or between 0.2 mg/kg and 5 mg/kg of the compound that binds to CD131. In one example, the method comprises administering about 0.5-2.0 mg/kg of the compound that binds to CD131.

In one example, eosinophil levels are measured in the subject. For example, eosinophil levels are measured in the subject's sputum. In another example, eosinophil levels are measured in the subject's blood.

In one example, a method described herein further comprises determining a peripheral blood eosinophil count and/or a peripheral-blood differential eosinophil count in the subject and/or a proportion of eosinophils in the subject's sputum. For example, the method further comprises determining a peripheral blood eosinophil count in the subject. In another example, the method further comprises determining a peripheral-blood differential eosinophil count in the subject. In a further example, the method further comprises determining a proportion of eosinophils in the subject's sputum.

In one example, the subject has a high sputum and/or high blood eosinophil count. For example, the subject has an absolute eosinophil count of at least 200 cells per microliter of blood and/or the proportion of eosinophils in the subject's sputum is at least 1.25%. In one example, the subject has an absolute eosinophil count of at least 500 cells per microliter of blood and/or the proportion of eosinophils in the subject's sputum is at least 3.00%

In one example, the subject has a high blood eosinophil count. In one example, the subject has an absolute eosinophil count of greater than 200 cells per microliter of blood. For example, the subject has an absolute eosinophil count of greater than 200 cells, or greater than 300 cells, or greater than 400 cells, or greater than 500 cells per microliter of blood. In one example, the subject has an absolute eosinophil count of greater than 500 cells per microliter of blood.

In one example, the subject has a high sputum count. In one example, the proportion of eosinophils in the subject's sputum is at least 1.25%. For example, the proportion of eosinophils in the subject's sputum is at least 1.25%, or at least 1.5%, or at least 1.75%, or at least 2.00%, or at least 2.25%, or at least 2.5%, or at least 2.75%, or at least 3.00%. In one example, the proportion of eosinophils in the subject's sputum is at least 3.00%.

The present disclosure further provides methods of diagnosing and/or detecting eosinophilia in a subject suffering from smoking-related chronic obstructive pulmonary disease (COPD), the method comprising determining a peripheral blood eosinophil count and/or a peripheral-blood differential eosinophil count in the subject.

### Key to Sequence Listing

SEQ ID NO 1: amino acid sequence of Homo sapiens CD131
SEQ ID NO 2: amino acid sequence of Homo sapiens CD131
SEQ ID NO 3: amino acid sequence of Homo sapiens GCS-F receptor
SEQ ID NO 4: amino acid sequence of Homo sapiens IL-5 receptor
SEQ ID NO 5: amino acid sequence of V_{L} chain of antibody 9A2
SEQ ID NO 6: amino acid sequence of V_{L} chain of antibody 9A2-VR1
SEQ ID NO 7: amino acid sequence of V_{L} chain of antibody 9A2-VR2
SEQ ID NO 8: amino acid sequence of V_{L} chain of antibody 9A2-VR3
SEQ ID NO 9: amino acid sequence of V_{L} chain of antibody 9A2-VR4
SEQ ID NO 10: amino acid sequence of V_{L} chain of antibody 9A2-VR5
SEQ ID NO 11: amino acid sequence of V_{L} chain of antibody 9A2-VR6
SEQ ID NO 12: amino acid sequence of V_{L} chain of antibody 9A2-VR8
SEQ ID NO 13: amino acid sequence of V_{L} chain of antibody 9A2-VR9
SEQ ID NO 14: amino acid sequence of V_{L} chain of antibody 9A2-VR11
SEQ ID NO 15: amino acid sequence of V_{L} chain of antibody 9A2-VR12
SEQ ID NO 16: amino acid sequence of V_{L} chain of antibody 9A2-VR13
SEQ ID NO 17: amino acid sequence of V_{L} chain of antibody 9A2-VR14
SEQ ID NO 18: amino acid sequence of V_{L} chain of antibody 9A2-VR16
SEQ ID NO 19: amino acid sequence of V_{L} chain of antibody 9A2-VR19
SEQ ID NO 20: amino acid sequence of V_{H} chain of antibody 9A2
SEQ ID NO 21: amino acid sequence of V_{H} chain of antibody 9A2-VR20
SEQ ID NO 22: amino acid sequence of V_{H} chain of antibody 9A2-VR21
SEQ ID NO 23: amino acid sequence of V_{H} chain of antibody 9A2-VR22
SEQ ID NO 24: amino acid sequence of V_{H} chain of antibody 9A2-VR23
SEQ ID NO 25: amino acid sequence of V_{H} chain of antibody 9A2-VR24
SEQ ID NO 26: amino acid sequence of V_{H} chain of antibody 9A2-VR26
SEQ ID NO 27: amino acid sequence of V_{H} chain of antibody 9A2-VR27
SEQ ID NO 28: amino acid sequence of V_{H} chain of antibody 9A2-VR28
SEQ ID NO 29: amino acid sequence of V_{H} chain of antibody 9A2-VR31
SEQ ID NO 30: amino acid sequence of V_{H} chain of antibody 9A2-VR32
SEQ ID NO 31: amino acid sequence of V_{H} chain of antibody 9A2-VR33
SEQ ID NO 32: amino acid sequence of V_{H} chain of antibody 9A2-VR34
SEQ ID NO 33: amino acid sequence of V_{H} chain of antibody 9A2-VR35
SEQ ID NO 34: amino acid sequence of V_{H} chain of antibody 9A2-VR36
SEQ ID NO 35: amino acid sequence of V_{H} chain of antibody 9A2-VR37
SEQ ID NO 36: amino acid sequence of V_{H} chain of antibody 9A2-VR38
SEQ ID NO 37: amino acid sequence of V_{H} chain of antibody 9A2-VR39
SEQ ID NO 38: amino acid sequence of V_{H} chain of antibody 9A2-VR40
SEQ ID NO 39: amino acid sequence of V_{H} chain of antibody 9A2-VR41
SEQ ID NO 40: amino acid sequence of V_{H} chain of antibody 9A2-VR42
SEQ ID NO 41: amino acid sequence of V_{H} chain of antibody 9A2-VR43
SEQ ID NO 42: amino acid sequence of V_{H} chain of antibody 9A2-VR44
SEQ ID NO 43: amino acid sequence of V_{H} chain of antibody 9A2-VR45
SEQ ID NO 44: amino acid sequence of V_{H} chain of antibody 9A2-VR46
SEQ ID NO 45: amino acid sequence of V_{H} chain of antibody 9A2-VR47
SEQ ID NO 46: amino acid sequence of V_{H} chain of antibody 9A2-VR48
SEQ ID NO 47: amino acid sequence of V_{H} chain of antibody 9A2-VR49
SEQ ID NO 48: amino acid sequence of V_{H} chain of antibody 9A2-VR50
SEQ ID NO 49: amino acid sequence of V_{H} chain of antibody 9A2-VR24.04
SEQ ID NO 50: amino acid sequence of V_{H} chain of antibody 9A2-VR24.07
SEQ ID NO 51: amino acid sequence of V_{H} chain of antibody 9A2-VR24.10
SEQ ID NO 52: amino acid sequence of V_{H} chain of antibody 9A2-VR24.12
SEQ ID NO 53: amino acid sequence of V_{H} chain of antibody 9A2-VR24.19
SEQ ID NO 54: amino acid sequence of V_{H} chain of antibody 9A2-VR24.24
SEQ ID NO 55: amino acid sequence of V_{H} chain of antibody 9A2-VR24.76
SEQ ID NO 56: amino acid sequence of V_{H} chain of antibody 9A2-VR24.78
SEQ ID NO 57: amino acid sequence of V_{H} chain of antibody 9A2-VR24.81
SEQ ID NO 58: amino acid sequence of V_{H} chain of antibody 9A2-VR24.82
SEQ ID NO 59: amino acid sequence of V_{H} chain of antibody 9A2-VR24.84
SEQ ID NO 60: amino acid sequence of V_{H} chain of antibody 9A2-VR24.87
SEQ ID NO 61: amino acid sequence of V_{H} chain of antibody 9A2-VR24.91
SEQ ID NO 62: amino acid sequence of V_{H} chain of antibody 9A2-VR24.93
SEQ ID NO 63: amino acid sequence of V_{H} chain of antibody 9A2-VR24.27
SEQ ID NO 64: amino acid sequence of V_{H} chain of antibody 9A2-VR24.29
SEQ ID NO 65: amino acid sequence of V_{H} chain of antibody 9A2-VR24.30
SEQ ID NO 66: amino acid sequence of V_{H} chain of antibody 9A2-VR24.33
SEQ ID NO 67: amino acid sequence of V_{H} chain of antibody 9A2-VR24.44
SEQ ID NO 68: amino acid sequence of V_{H} chain of antibody 9A2-VR24.97
SEQ ID NO 69: amino acid sequence of V_{H} chain of antibody 9A2-VR24.98
SEQ ID NO 70: amino acid sequence of V_{H} chain of antibody 9A2-VR24.102
SEQ ID NO 71: amino acid sequence of V_{H} chain of antibody 9A2-VR24.107
SEQ ID NO 72: amino acid sequence of V_{H} chain of antibody 9A2-VR24.110
SEQ ID NO 73: amino acid sequence of V_{H} chain of antibody 9A2-VR24.111
SEQ ID NO 74: amino acid sequence of V_{H} chain of antibody 9A2-VR24.55
SEQ ID NO 75: amino acid sequence of V_{H} chain of antibody 9A2-VR24.56
SEQ ID NO 76: amino acid sequence of V_{H} chain of antibody 9A2-VR24.57
SEQ ID NO 77: amino acid sequence of V_{H} chain of antibody 9A2-VR24.122
SEQ ID NO 78: amino acid sequence of V_{H} chain of antibody 9A2-VR24.124
SEQ ID NO 79: amino acid sequence of V_{H} chain of antibody 9A2-VR24.131
SEQ ID NO 80: amino acid sequence of V_{H} chain of antibody 9A2-VR39.01
SEQ ID NO 81: amino acid sequence of V_{H} chain of antibody 9A2-VR39.02
SEQ ID NO 82: amino acid sequence of V_{H} chain of antibody 9A2-VR39.04
SEQ ID NO 83: amino acid sequence of V_{H} chain of antibody 9A2-VR39.05
SEQ ID NO 84: amino acid sequence of V_{H} chain of antibody 9A2-VR39.06
SEQ ID NO 85: amino acid sequence of V_{H} chain of antibody 9A2-VR39.11
SEQ ID NO 86: amino acid sequence of V_{H} chain of antibody 9A2-VR39.12
SEQ ID NO 87: amino acid sequence of V_{H} chain of antibody 9A2-VR39.16
SEQ ID NO 88: amino acid sequence of V_{H} chain of antibody 9A2-VR39.17
SEQ ID NO 89: amino acid sequence of V_{H} chain of antibody 9A2-VR39.18
SEQ ID NO 90: amino acid sequence of V_{H} chain of antibody 9A2-VR39.19
SEQ ID NO 91: amino acid sequence of V_{H} chain of antibody 9A2-VR39.21
SEQ ID NO 92: amino acid sequence of V_{H} chain of antibody 9A2-VR39.22
SEQ ID NO 93: amino acid sequence of V_{H} chain of antibody 9A2-VR39.23
SEQ ID NO 94: amino acid sequence of V_{H} chain of antibody 9A2-VR39.24
SEQ ID NO 95: amino acid sequence of V_{H} chain of antibody 9A2-VR39.97
SEQ ID NO 96: amino acid sequence of V_{H} chain of antibody 9A2-VR39.98
SEQ ID NO 97: amino acid sequence of V_{H} chain of antibody 9A2-VR39.102
SEQ ID NO 98: amino acid sequence of V_{H} chain of antibody 9A2-VR39.103
SEQ ID NO 99: amino acid sequence of V_{H} chain of antibody 9A2-VR39.105
SEQ ID NO 100: amino acid sequence of V_{H} chain of antibody 9A2-VR39.109
SEQ ID NO 101: amino acid sequence of V_{H} chain of antibody 9A2-VR39.110
SEQ ID NO 102: amino acid sequence of V_{H} chain of antibody 9A2-VR39.111
SEQ ID NO 103: amino acid sequence of V_{H} chain of antibody 9A2-VR39.112
SEQ ID NO 104: amino acid sequence of V_{H} chain of antibody 9A2-VR39.116
SEQ ID NO 105: amino acid sequence of V_{H} chain of antibody 9A2-VR39.27
SEQ ID NO 106: amino acid sequence of V_{H} chain of antibody 9A2-VR39.28
SEQ ID NO 107: amino acid sequence of V_{H} chain of antibody 9A2-VR39.46
SEQ ID NO 108: amino acid sequence of V_{H} chain of antibody 9A2-VR39.122
SEQ ID NO 109: amino acid sequence of V_{H} chain of antibody 9A2-VR39.139
SEQ ID NO 110: amino acid sequence of V_{H} chain of antibody 9A2-VR39.140
SEQ ID NO 111: amino acid sequence of V_{H} chain of antibody 9A2-VR39.148
SEQ ID NO 112: amino acid sequence of V_{H} chain of antibody 9A2-VR39.162
SEQ ID NO 113: amino acid sequence of V_{H} chain of antibody 9A2-VR39.77
SEQ ID NO 114: amino acid sequence of V_{H} chain of antibody 9A2-VR39.93
SEQ ID NO 115: amino acid sequence of V_{H} chain of antibody 9A2-VR39.174
SEQ ID NO 116: amino acid sequence of V_{H} chain of antibody 9A2-VR39.177
SEQ ID NO 117: amino acid sequence of soluble Homo sapiens CD131 comprising a C-terminal 6×His tag and comprising the substitution N37A
SEQ ID NO 118: amino acid sequence of soluble Homo sapiens CD131 comprising a C-terminal 6×His tag and comprising the substitution D38A
SEQ ID NO 119: amino acid sequence of soluble Homo sapiens CD131 comprising a C-terminal 6×His tag and comprising the substitution Y39A
SEQ ID NO 120: amino acid sequence of soluble Homo sapiens CD131 comprising a C-terminal 6×His tag and comprising the substitution T40A
SEQ ID NO 121: amino acid sequence of soluble Homo sapiens CD131 comprising a C-terminal 6×His tag and comprising the substitution S41A
SEQ ID NO 122: amino acid sequence of soluble Homo sapiens CD131 comprising a C-terminal 6×His tag and comprising the substitution H42A
SEQ ID NO 123: amino acid sequence of soluble Homo sapiens CD131 comprising a C-terminal 6×His tag and comprising the substitution S102A
SEQ ID NO 124: amino acid sequence of soluble Homo sapiens CD131 comprising a C-terminal 6×His tag and comprising the substitution F103A
SEQ ID NO 125: amino acid sequence of soluble Homo sapiens CD131 comprising a C-terminal 6×His tag and comprising the substitution V104A
SEQ ID NO 126: amino acid sequence of soluble Homo sapiens CD131 comprising a C-terminal 6×His tag and comprising the substitution V105A
SEQ ID NO 127: amino acid sequence of soluble Homo sapiens CD131 comprising a C-terminal 6×His tag and comprising the substitution T106A
SEQ ID NO 128: amino acid sequence of soluble Homo sapiens CD131 comprising a C-terminal 6×His tag and comprising the substitution D107A
SEQ ID NO 129: amino acid sequence of soluble Homo sapiens CD131 comprising a C-terminal 6×His tag and comprising the substitution V108A
SEQ ID NO 130: amino acid sequence of soluble Homo sapiens CD131 comprising a C-terminal 6×His tag and comprising the substitution N337A
SEQ ID NO 131: amino acid sequence of soluble Homo sapiens CD131 comprising a C-terminal 6×His tag and comprising the substitution I338A
SEQ ID NO 132: amino acid sequence of soluble Homo sapiens CD131 comprising a C-terminal 6×His tag and comprising the substitution Q339A
SEQ ID NO 133: amino acid sequence of soluble Homo sapiens CD131 comprising a C-terminal 6×His tag and comprising the substitution M340A
SEQ ID NO 134: amino acid sequence of soluble Homo sapiens CD131 comprising a C-terminal 6×His tag and comprising the substitution K362A
SEQ ID NO 135: amino acid sequence of soluble Homo sapiens CD131 comprising a C-terminal 6×His tag and comprising the substitution M363A
SEQ ID NO 136: amino acid sequence of soluble Homo sapiens CD131 comprising a C-terminal 6×His tag and comprising the substitution R364A
SEQ ID NO 137: amino acid sequence of soluble Homo sapiens CD131 comprising a C-terminal 6×His tag and comprising the substitution Y365A
SEQ ID NO 138: amino acid sequence of soluble Homo sapiens CD131 comprising a C-terminal 6×His tag and comprising the substitution E366A
SEQ ID NO 139: amino acid sequence of soluble Homo sapiens CD131 comprising a C-terminal 6×His tag and comprising the substitution H367A
SEQ ID NO 140: amino acid sequence of soluble Homo sapiens CD131 comprising a C-terminal 6×His tag and comprising the substitution I368A
SEQ ID NO 141: amino acid sequence of soluble Homo sapiens CD131 comprising a C-terminal 6×His tag and comprising the substitution D369A
SEQ ID NO 142: amino acid sequence of soluble Homo sapiens CD131 comprising a C-terminal 6×His tag and comprising the substitution R418A
SEQ ID NO 143: amino acid sequence of soluble Homo sapiens CD131 comprising a C-terminal 6×His tag and comprising the substitution T419A
SEQ ID NO 144: amino acid sequence of soluble Homo sapiens CD131 comprising a C-terminal 6×His tag and comprising the substitution n G420A
SEQ ID NO 145: amino acid sequence of soluble Homo sapiens CD131 comprising a C-terminal 6×His tag and comprising the substitution Y421A
SEQ ID NO 146: amino acid sequence of soluble Homo sapiens CD131 comprising a C-terminal 6×His tag and comprising the substitution N422A
SEQ ID NO 147: amino acid sequence of soluble Homo sapiens CD131 comprising a C-terminal 6×His tag and comprising the substitution G423A
SEQ ID NO 148: amino acid sequence of soluble Homo sapiens CD131 comprising a C-terminal 6×His tag and comprising the substitution I424A
SEQ ID NO 149: nucleotide acid sequence encoding trimer oligonucleotide 9A2 L1.1
SEQ ID NO 150: nucleotide acid sequence encoding trimer oligonucleotide 9A2 L3.1
SEQ ID NO 151: nucleotide acid sequence encoding trimer oligonucleotide 9A2 L3.2
SEQ ID NO 152: nucleotide acid sequence encoding trimer oligonucleotide 9A2 H1.1
SEQ ID NO 153: nucleotide acid sequence encoding trimer oligonucleotide 9A2 H2.1
SEQ ID NO 154: nucleotide acid sequence encoding trimer oligonucleotide 9A2 H3.1
SEQ ID NO 155: nucleotide acid sequence encoding trimer oligonucleotide 9A2 H3.2
SEQ ID NO 156: nucleotide acid sequence encoding trimer oligonucleotide 9A2-VR24-H2.1
SEQ ID NO 157: nucleotide acid sequence encoding trimer oligonucleotide 9A2-VR24-H2.2
SEQ ID NO 158: nucleotide acid sequence encoding trimer oligonucleotide 9A2-VR24-H2.3
SEQ ID NO 159: nucleotide acid sequence encoding trimer oligonucleotide 9A2-VR39-H1.1
SEQ ID NO 160: nucleotide acid sequence encoding trimer oligonucleotide 9A2-VR39-H1.2
SEQ ID NO 161: nucleotide acid sequence encoding trimer oligonucleotide 9A2-VR39-H2.2
SEQ ID NO 162: nucleotide acid sequence encoding trimer oligonucleotide 9A2-VR39-H2.3
SEQ ID NO 163: amino acid sequence of V_{H} chain of stop template of 9A2 H1.1
SEQ ID NO 164: amino acid sequence of V_{H} chain of stop template of 9A2 H2.1
SEQ ID NO 165: amino acid sequence of V_{H} chain of stop template of 9A2 H3.1
SEQ ID NO 166: amino acid sequence of V_{H} chain of stop template of 9A2 H3.2
SEQ ID NO 167: amino acid sequence of V_{L} chain of stop template of 9A2 L1.1
SEQ ID NO 168: amino acid sequence of V_{L} chain of stop template of 9A2 L3.1
SEQ ID NO 169: amino acid sequence of V_{L} chain of stop template of 9A2 L3.2
SEQ ID NO 170: amino acid sequence of V_{H} chain of stop template of 9A2 VR24-H2.1
SEQ ID NO 171: amino acid sequence of V_{H} chain of stop template of 9A2 VR24-H2.2
SEQ ID NO 172: amino acid sequence of V_{H} chain of stop template of 9A2 VR24-H2.3
SEQ ID NO 173: amino acid sequence of V_{H} chain of stop template of 9A2 VR39-H1.1
SEQ ID NO 174: amino acid sequence of V_{H} chain of stop template of 9A2 VR39-H1.2
SEQ ID NO 175: amino acid sequence of V_{H} chain of stop template of 9A2 VR39-H2.2
SEQ ID NO 176: amino acid sequence of V_{H} chain of stop template of 9A2 VR39-H2.3
SEQ ID NO 177: amino acid sequence of consensus of V_{L} chain of 9A2 and derivatives
SEQ ID NO 178: amino acid sequence of consensus of CDR1 of V_{L} chain of 9A2 and derivatives
SEQ ID NO 179: amino acid sequence of consensus of CDR3 of V_{L} chain of 9A2 and derivatives
SEQ ID NO 180: amino acid sequence of consensus of V_{H} chain of 9A2 and derivatives
SEQ ID NO 181: amino acid sequence of consensus of CDR1 of V_{H} chain of 9A2 and derivatives
SEQ ID NO 182: amino acid sequence of consensus of CDR2 of V_{H} chain of 9A2 and derivatives
SEQ ID NO 183: amino acid sequence of consensus of CDR3 of V_{H} chain of 9A2 and derivatives
SEQ ID NO 184: amino acid sequence of consensus of V_{H} chain of 9A2-VR24 and derivatives
SEQ ID NO 185: amino acid sequence of consensus of CDR1 of V_{H} chain of 9A2-VR24 and derivatives
SEQ ID NO 186: amino acid sequence of consensus of CDR2 of V_{H} chain of 9A2-VR24 and derivatives
SEQ ID NO 187: amino acid sequence of consensus of CDR3 of V_{H} chain of 9A2-VR24 and derivatives
SEQ ID NO 188: amino acid sequence of consensus of V_{H} chain of 9A2-VR39 and derivatives
SEQ ID NO 189: amino acid sequence of consensus of CDR1 of V_{H} chain of 9A2-VR39 and derivatives
SEQ ID NO 190: amino acid sequence of consensus of CDR2 of V_{H} chain of 9A2-VR39 and derivatives
SEQ ID NO 191: amino acid sequence of consensus of CDR3 of V_{H} chain of 9A2-VR39 and derivatives
SEQ ID NO: 192: amino acid sequence of soluble Homo sapiens CD131 comprising a C-terminal 6×His tag
SEQ ID NO: 193: amino acid sequence of V_{H} chain of 9A2-VR24 HCDR2 mutants
SEQ ID NO: 194: amino acid sequence of antibody 9A2 heavy chain
SEQ ID NO: 195: amino acid sequence of antibody 9A2 light chain
SEQ ID NO: 196: amino acid sequence of stabilized IgG4 heavy chain constant region
SEQ ID NO: 197: amino acid sequence of kappa light chain constant region

### BRIEF DESCRIPTION OF THE DRAWINGS

FIG. 1 is a summary of the flow cytometry gating strategy. Cell debris and aggregates were first excluded, from which CD45 positive leukocytes were gated. Neutrophils were defined as Ly6G⁺CD11c^{-/low} and from the non-neutrophil population, CD11b⁻CD11c⁻ lymphocytes were excluded. The non-lymphocytic population was gated for F4/80⁺ cells, which were then further gated for alveolar macrophages (AMs, Sig lecF⁺CD11c⁺), interstitial macrophages (IMs, SiglecF⁻CD11b⁺CD11c⁻) and exudative macrophages (ExMs, SiglecF⁻CD11b⁺CD11c⁺). Eosinophils were identified as CD11c⁻ SiglecF⁺CD11b).

FIG. 2 is a series of graphical representations showing hβcTg mice and their wild type (WT) counterparts following exposure to 4-day cigarette smoke or room air and culled on the fifth day. Final body weights showed a similar weight loss in both genotypes post CS exposure (A). Total BAL cells (B), mainly consisting of macrophages (C), were also increased in response to cigarette smoke exposure in both genotypes, but to a lower level in hβcTg mice.

FIG. 3 is a series of graphical representations showing cigarette smoke (CS) induced weight loss and inflammation in hβcTg mice (A) Mice were exposed to 11-consecutive days of room air (sham) or cigarette smoke (CS) and culled the following day. (B) Body weight was monitored over the experimental protocol demonstrating a reduction in CS-exposed mice mice at the end of the protocol. (C) Total white blood cell (WBC), (D) blood monocytes, (E) blood neutrophils and (F) blood lymphocytes were quantified by haematological analysis. (G) Total bronchoalveolar lavage (BAL) cell counts were determined, and differential cell counts performed on cytospots to quantify (H) BAL macrophages, (I) BAL neutrophils and (J) BAL lymphocytes. Flow cytometry was also performed to quantify lung (K), neutrophils, (L) interstitial macrophages, (M) exudative macrophages, and (N) eosinophils. Sham; n=5; CS n=6. *p<0.05 student's t-test vs. sham.

FIG. 4 is a series of graphical representations showing cigarette smoke (CS)-induced lung injury and oedema in the bronchoalveolar lavage fluid (BALF). (A) Aggregated lung injury score (0-9) quantified from whole H&E-stained lung sections. 0=no injury; 9=severe injury. Lung oedema and injury markers were quantified in the BAL fluid including (B) albumin and (C) lactate dehydrogenase (LDH). Markers of leukocyte degranulation were quantified in BAL fluid including (D) double-stranded DNA (dsDNA) and (E) gelatinase activity. Sham n=5; CS n=6. *p<0.05 student's t-test against sham.

FIG. 5 is a series of graphical representations showing acute cigarette smoke promotes expression of multiple immune regulators. RTqPCR on lung tissue including (A) the monocyte chemokine CCL2, (B) MMP-12 and (C) Fizz1. Neutrophil recruitment markers (D) IL-1α, (E) CXCL1, and (F) CXCL2 were also quantified. The IL-23/IL17A axis was also investigated by quantifying (G) IL-17A, (H) IL-23 and (I) GM-CSF expression. Sham n=5; CS n=6. *p<0.05 student's t-test against sham.

FIG. 6 is a series of graphical representations showing CSL311 reduced weight loss and inflammation caused by CS exposure. (A) Transgenic hβcTg mice were exposed to 11 days of cigarette smoke and treated with 4 doses of CSL311 (iv) as detailed in the schematic. (B) Body weight was monitored, where CSL311 significantly reduced body weight loss caused by CS exposure compared to isotype control. Haematological analysis of blood included (C) total white blood cell count, (D) blood monocytes, (E) blood neutrophils and (F) blood lymphocytes. (G) Total bronchoalveolar lavage (BAL) cell counts were determined, and differential cell counts performed on cytospots to quantify BAL (H) macrophages, (I) neutrophils and (J) lymphocytes. Flow cytometry was also performed to quantify lung (K) neutrophils, (L) interstitial macrophages, (M) exudative macrophages, (N) and eosinophils. ISO n=5; CSL311 n=6. *p<0.05 student's t-test vs. ISO.

FIG. 7 is a series of graphical representations showing CSL311 reduced lung injury and oedema caused by acute cigarette smoke exposure. (A) Aggregated lung injury score (0-9) quantified from whole H&E-stained lung sections. 0=no injury; 9=severe injury. Lung oedema and injury markers were quantified in the BAL fluid including (B) albumin and (C) lactate dehydrogenase (LDH). Markers of leukocyte degranulation were quantified in BAL fluid including (D) double-stranded DNA (dsDNA) and (E) gelatinase activity. ISO n=5; CSL311 n=6. *p<0.05 student's t-test against ISO.

FIG. 8 is a series of graphical representations showing CSL311 inhibited expression of multiple inflammatory mediators performed on lung tissue including (A) the monocyte chemokine CCL2, (B) MMP-12 and (C) Fizz1. Neutrophil recruitment mediators, (D) IL-1α, (E) CXCL1, and (F) CXCL2 were also quantified. The IL-23/IL17A axis was also investigated by quantifying (G) IL-17A, (H) IL-23 and (I) GM-CSF. ISO n=5; CSL311 n=6. *p<0.05 student's t-test against ISO.

FIG. 9 is a series of graphical representations showing CSL311 reduced (A) lung tissue collagen gene expression (COL1A1) and (B) lung tissue mucin gene expression (MUC5AC) induced by 11 days of cigarette smoke exposure in hβcTg mice.

FIG. 10 is a series of graphical representations showing effect of CSL311 treatment on (A) BALF cells (B) BALF macrophages (C) BALF neutrophils (D) BALF eosinophils (E) lung neutrophils (F) lung CD11b+ macrophages and (G) lung eosinophils in hβcTg mice exposed to HDM and PPE (ACO). A separate cohort of mice were challenged to inhaled methacholine (Mch) to measure total respiratory resistance (Rrs, H) and airway resistance (Rn, J), where maximal Mch responses were also presented (Rrs Max, I; Rn Max, K). Airspace enlargement (emphysema) was measured by the mean linear intercept method (Lm) on H&E stained lung sections (L). A schematic of the treatment protocol is depicted in M. n=6 for each group. * p<0.05, ** p<0.01, *** p<0.001 One-way ANOVA with Tukey's post hβc analysis.

FIG. 11 is a series of graphical representations showing STAT5 phosphorylation in response to GM-CSF stimulation and subsequent blocking of activation by CSL311 in (A) monocytes (B) basophils (C) eosinophils and (D) neutrophils.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only.

Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

For the purposes of nomenclature only and not limitation an exemplary sequence of a human CD131 (pre-CD131) is set out in NCBI Reference Sequence: NP_000386.1 and NCBI Genbank Accession Number P32927 (and set out in SEQ ID NO: 1). A sequence of a mature human CD131 lacks amino acids 1 to 16 of SEQ ID NO: 1. Positions of amino acids are often referred to herein by reference to pre-CD131. The positions in mature CD131 is readily determined by accounting for the signal sequence (amino acids 1-16 in the case of SEQ ID NO: 1). The sequence of CD131 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human CD131 may be abbreviated to hCD131. Reference to soluble CD131 refers to polypeptides comprising the extracellular region of CD131, e.g., amino acids 17 to 438 of SEQ ID NO: 1.

Reference herein to CD131 includes native forms of CD131 and mutant forms thereof retaining an ability to bind to CD131 (e.g., hCD131) and induce signaling. CD131 is also known as “CSF2RB” and “cytokine receptor common subunit beta” and “β (beta) common receptor” (abbreviated as “βCR” or “βc”).

A “compound”, as contemplated by the present disclosure, can take any of a variety of forms including natural compounds, chemical small molecule compounds or biological compounds or macromolecules. Exemplary compounds include an antibody or a protein comprising an antigen binding fragment of an antibody, a nucleic acid, a polypeptide, a peptide, and a small molecule.

The terms “reduce” or “reducing” as used herein in reference to cells, e.g., eosinophils, refers to administering a compound described herein to stop or hinder the development, differentiation and/or maturation, accumulation, migration and/or recruitment of cells, e.g., eosinophils, in the subject.

The terms “deplete” or “depleting” as used herein in reference to eosinophils refers to administering a compound described herein to stop or hinder the survival of eosinophils in the subject. For example, the compound may induce eosinophil cell death by e.g., apoptosis (programmed cell death), cytolysis, autophagy and/or netosis. The compound may act by blocking pro-survival signals (e.g. IL-3-, IL-5- and/or GM-CSF-mediated signals) to cells thereby leading to cell death, e.g. by apoptosis.

As used herein, the terms “treating”, “treat” or “treatment” include administering a compound described herein to reduce, prevent, or eliminate at least one symptom of a specified disease or condition.

As used herein, the terms “preventing”, “prevent” or “prevention” include administering a compound described herein to thereby stop or hinder the development of at least one symptom of a condition, e.g., before that symptom is fully developed in the subject.

As used herein, the phrase “delay progression of” include administering a compound described herein to thereby reduce or slow the development of at least one symptom of a condition.

The terms “reduce” and “prevent an increase in” are used herein to refer to a lower amount of any of the genes and/or factors recited herein, relative to either the amount in the subject prior to administration of the compound that binds to CD131, or relative to the amount in a corresponding control subject. For instance, the control subject may be a subject who receives a placebo and/or a standard of care therapy, rather than the compound that binds to CD131.

As used herein, the term “subject” shall be taken to mean any animal including humans, for example a mammal. Exemplary subjects include but are not limited to humans and non-human primates. In one example, the subject is a human.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. In some examples, the protein is a fusion protein. As used herein, a “fusion protein” is a protein comprising at least two domains that have been joined so that they are translated as a single unit, producing a single protein.

The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some examples, an antigen binding site is or comprises a V_{H} or a V_{L} or a Fv. In some examples, the antigen binding site comprises one or more CDRs of an antibody. The antigen binding site need not be in the context of an entire antibody, e.g., it can be in isolation (e.g., a domain antibody) or in another form, e.g., as described herein, such as a scFv.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-(CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to CD131 (e.g., hCD131 or a polypeptide comprising a region thereof, e.g., a polypeptide comprising a sequence set forth in SEQ ID NO: 191) with materially greater affinity (e.g., 20 fold or 40 fold or 60 fold or 80 fold to 100 fold or 150 fold or 200 fold) than it does to other cytokine receptors or to antigens commonly recognized by polyreactive natural antibodies (i.e., by naturally occurring antibodies known to bind a variety of antigens naturally found in humans). In an example of the present disclosure, a compound “specifically binds” to one form of hCD131 or a polypeptide comprising a region thereof (e.g., the extracellular region of hCD131) or a polypeptide comprising a sequence set forth in SEQ ID NO: 191 with an affinity at least 1.5 fold or 2 fold or greater (e.g., 5 fold or 10 fold or 20 fold r 50 fold or 100 fold or 200 fold) than it does to a mutant form of SEQ ID NO: 191 comprising a sequence set forth in SEQ ID NO: 119, 124, 131 or 137. Reference to “binding” provides explicit support for the term “specific binding” and vice versa.

A protein or antibody may be considered to “preferentially bind” to a polypeptide if it binds that polypeptide with a dissociation constant (K_{D}) that is less than the protein's or antibody's K_{D} for another polypeptide. In one example, a protein or antibody is considered to preferentially bind to a polypeptide if it binds the polypeptide with an affinity (i.e., K_{D}) that is at least about 20 fold or 40 fold or 60 fold or 80 fold or 100 fold or 120 fold or 140 fold or 160 fold more than the protein's or antibody's K_{D} for another polypeptide.

For the purposes of clarification and as will be apparent to the skilled artisan based on the exemplified subject matter herein, reference to “affinity” in this specification is a reference to K_{D} of a protein or antibody.

For the purposes of clarification and as will be apparent to the skilled artisan based on the description herein, reference to “a K_{D} of X nM or less” will be understood to mean that the numerical value of the K_{D} is equal to X nM or is lower in numerical value. As a skilled person would understand a lower numerical value of a K_{D} corresponds to a higher (i.e., stronger) affinity, i.e., an affinity of 2 nM is stronger than an affinity of 3 nM.

An “IC₅₀ of at least about” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

As used herein, the term “epitope” (syn. “antigenic determinant”) shall be understood to mean a region of CD131 to which a compound comprising an antigen binding site of an antibody binds. This term is not necessarily limited to the specific residues or structure to which the compound makes contact. For example, this term includes the region spanning amino acids contacted by the compound and/or 5-10 or 2-5 or 1-3 amino acids outside of this region. In some examples, the epitope comprises a series of discontinuous amino acids that are positioned close to one another when CD131 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a compound (i.e., CD131-binding protein or antibody) of the disclosure (or an antigen binding site thereof) reduces or prevents binding of a recited antibody or protein to CD131. This may be due to the protein (or antigen binding site) and antibody binding to the same or an overlapping epitope. It will be apparent from the foregoing that the compound need not completely inhibit binding of the antibody, rather it need only reduce binding by a statistically significant amount, for example, by at least about 10% or 20% or 30% or 40% or 50% or 60% or 70% or 80% or 90% or 95%. Preferably, the compound reduces binding of the protein or antibody to CD131 by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to CD131 either in the presence or absence of the compound. If less antibody binds in the presence of the compound than in the absence of the compound, the compound is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a compound (or protein comprising an antigen binding site thereof) that binds to one epitope to competitively inhibit the binding of a compound (or protein comprising an antigen binding site) that binds to the other epitope. For example, the “overlapping” epitopes share at least 1 or 2 or 3 or 4 or 5 or 6 or 7 or 8 or 9 or 20 amino acids.

As used herein, the term “neutralize” shall be taken to mean that a compound that binds CD131 is capable of blocking, reducing or preventing CD131-mediated signaling in a cell by IL-3, IL-5 and/or GM-CSF. Methods for determining neutralization are known in the art and/or described herein.

### Treatment of Eosinophil-Associated COPD

The methods described herein provide methods of treating, preventing and/or delaying progression of eosinophilia in a subject suffering from COPD by administering a compound that binds to CD131 and/or inhibits signalling of IL-3, IL-5 and GM-CSF.

### Eosinophilia

The present disclosure provides methods of depleting and/or reducing eosinophils in a subject suffering from an eosinophil-associated COPD.

The present disclosure also provides methods of treating, preventing and/or delaying progression of eosinophilia in a subject suffering from an eosinophil-associated COPD.

As used herein, the term “eosinophilia” refers to an increase in blood eosinophils in the subject greater than the upper limit of normal. The skilled person will recognise that the ‘normal’ limit of eosinophils is between about 0.0 and 6.0 percent of total white blood cells.

In one example, the subject is clinically diagnosed with eosinophilia. The skilled person will recognise that eosinophilia is clinically characterised by an absolute eosinophil count of greater than 500 cells per microliter of blood and/or a proportion of greater than 6.00% eosinophils in the blood and/or a proportion of greater than 3.00% eosinophils in the sputum. It will be apparent to the skilled person that the term eosinophilia also includes hypereosinophilia, which is clinically characterised by an absolute eosinophil count of greater than 1500 cells per microliter of blood that lasts for several months.

In one example, the subject has an absolute eosinophil count of greater than 500 cells per microliter of blood.

Methods of detecting eosinophilia will be apparent to the skilled person and/or are described herein. For example, eosinophilia is diagnosed on the basis of a blood test.

As used herein, the term “eosinophil-associated” in relation to COPD refers to a subject with COPD in addition to eosinophilia. For example, the subject has COPD and eosinophilia.

### Chronic Obstructive Pulmonary Disease (COPD)

As used herein, the term “chronic obstructive pulmonary disease (COPD)” refers to a chronic inflammatory lung disease that is characterised by persistent and progressive airway obstruction.

In one example, the subject suffers from COPD. In one example, the subject suffers from clinically diagnosed COPD.

In one example, the subject suffers from one or more clinically recognised symptoms of COPD, including for example:
chronic cough;
shortness of breath whilst doing everyday activities (dyspnea);
frequent respiratory infections;
increased sputum production;
blueness of the lips or fingernail beds (cyanosis);
fatigue; and
wheezing.

In one example, a subject with COPD is asymptomatic.

The term “COPD” as used herein encompasses all types of COPD including pre-COPD, early COPD (i.e., stage I COPD), moderate COPD (i.e., stage II COPD), severe COPD (i.e., stage III COPD) and very severe COPD (i.e., stage IV COPD or end-stage COPD).

Methods of diagnosing COPD will be apparent to the skilled person and/or are defined herein and include, for example, spirometry, computerized tomography (CT) scan, chest X-ray and/or an arterial blood gas test.

In one example, the COPD is diagnosed using spirometery. For example, the severity of COPD can be categorized based on the forced expiratory volume (FEV-1) and/or according to the Global Initiative for Chronic Obstructive Lung Disease (GOLD) system as follows:
GOLD 1: Mild/early COPD: FEV-1≥80%
GOLD 2: Moderate COPD: FEV-1 50-79%
GOLD 3: Severe COPD: FEV-1 30-49%
GOLD 4: Very severe COPD: FEV-1≤30%

In one example, the subject is at risk of developing COPD. For example, the subject is at risk of developing clinically diagnosed COPD. Risk factors for COPD will be apparent to the skilled person and/or are described herein.

In one example, the subject smokes and/or has a history of smoking and/or is exposed to second-hand smoke. For example, the COPD is smoking-related COPD.

In one example, the subject has, or suffers from, asthma-chronic obstructive pulmonary disease (COPD) overlap (ACO). For example, the subject has, or suffers from, eosinophil-associated asthma-chronic obstructive pulmonary disease (COPD) overlap.

In one example, the present disclosure provides a method for treating asthma-chronic obstructive pulmonary disease (COPD) overlap (ACO) in a subject comprising administering to the subject a compound that binds to or specifically binds to CD131 and neutralises signalling by IL-3, IL-5 and GM-CSF in a subject.

Compounds that Bind CD131

### Antibodies

In one example, a compound as described herein according to any example is a protein comprising an antigen binding site of an antibody. In some examples, the compound is an antibody.

Methods for generating antibodies are known in the art and/or described in Harlow and Lane (editors) Antibodies: A Laboratory Manual, Cold Spring Harbor Laboratory, (1988). Generally, in such methods CD131 or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. Nos. 6,300,064 and/or 5,885,793. For example, the present inventors have isolated fully human antibodies from a phage display library.

An antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

Exemplary human antibodies are described herein and include 9A2-VR24.29 (also, referred to as “CSL311”) described in WO 2017/088028 and BION-1 described in Sun et al. (1999) Blood 94:1943-1951 and/or proteins comprising variable regions thereof or derivatives thereof. These human antibodies provide an advantage of reduced immunogenicity in a human compared to non-human antibodies.

In one example, the antibody is a multispecific antibody. For instance, the compound that binds to CD131 may be a protein comprising an antigen binding site that binds to CD131 and a further antigen binding site that binds to a different antigen. Thus, in some examples, the antibody is a bispecific antibody.

### Antibody Binding Domain Containing Proteins

### Single-Domain Antibodies

In one example, a compound of the disclosure is a protein that is or comprises a single-domain antibody (which is used interchangeably with the term “domain antibody” or “dAb” or “nanobody”). A single-domain antibody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). In certain examples, a single-domain antibody is a human single-domain antibody (Domantis, Inc., Waltham, MA; see, e.g., U.S. Pat. No. 6,248,516).

In one example, the single-domain antibody is a V_{H}H fragment. V_{H}H fragments consist of the variable domain (V_{H}) of camelid heavy-chain antibodies, described below.

In one example, the single-domain antibody is a V_{NAR} fragment. V_{NAR} fragments consist of the variable domain (V_{H}) of heavy-chain antibodies from cartilaginous fish, described below.

### Diabodies, Triabodies, Tetrabodies

In one example, a protein of the disclosure is or comprises a diabody, triabody, tetrabody or higher order protein complex such as those described in WO98/044001 and/or WO94/007921.

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002/088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### De-Immunized Proteins

The present disclosure also contemplates a de-immunized antibody or protein. De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO2000/34317, WO2004/108158 and WO2004/064724.

Methods for introducing suitable mutations and expressing and assaying the resulting protein will be apparent to the skilled artisan based on the description herein.

### Mutations to Proteins

The present disclosure also contemplates mutant forms of a protein of the disclosure. For example, such a mutant protein comprises one or more conservative amino acid substitutions compared to a sequence set forth herein. In some examples, the protein comprises 30 or fewer or 20 or fewer or 10 or fewer, e.g., 9 or 8 or 7 or 6 or 5 or 4 or 3 or 2 conservative amino acid substitutions. A “conservative amino acid substitution” is one in which the amino acid residue is replaced with an amino acid residue having a similar side chain and/or hydropathicity and/or hydrophilicity.

In one example, a mutant protein has only, or not more than, one or two or three or four or five or six conservative amino acid changes when compared to a naturally occurring protein. Details of conservative amino acid changes are provided below. As the skilled person would be aware, e.g., from the disclosure herein, such minor changes can reasonably be predicted not to alter the activity of the protein.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

The present disclosure also contemplates non-conservative amino acid changes (e.g., substitutions) in a protein of the present disclosure, e.g., in a CDR, such as CDR3. In one example, the protein comprises fewer than 6 or 5 or 4 or 3 or 2 or 1 non-conservative amino acid substitutions, e.g., in a CDR3, such as in a CDR3.

The present disclosure also contemplates one or more insertions or deletions compared to a sequence set forth herein. In some examples, the protein comprises 10 or fewer, e.g., 9 or 8 or 7 or 6 or 5 or 4 or 3 or 2 insertions and/or deletions.

### Constant Regions

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present disclosure also contemplates additional modifications to an antibody or protein of the disclosure.

For example, the antibody comprises one or more amino acid substitutions that increase the half-life of the protein. For example, the antibody comprises a Fc region comprising one or more amino acid substitutions that increase the affinity of the Fc region for the neonatal Fc receptor (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

The protein may be a fusion protein. Thus, in one example, the protein additionally comprises albumin, a functional fragment or variant thereof. In one example, the albumin, functional fragment or variant thereof is serum albumin, such as human serum albumin. In one example, the albumin, functional fragment or variant thereof, comprises one or more amino acid substitutions, deletions or insertions, e.g., no more than 5 or 4 or 3 or 2 or 1 substitutions. Amino acid substitutions suitable for use in the present disclosure will be apparent to the skilled person and include naturally-occurring substitutions and engineered substitutions such as those described, for example, in WO2011/051489, WO2014/072481, WO2011/103076, WO2012/112188, WO2013/075066, WO2015/063611 and WO2014/179657.

In one example, the protein of the disclosure additionally comprises a soluble complement receptor or functional fragment or variant thereof. In one example, the protein additionally comprises a complement inhibitor.

### Exemplary CD131-Binding Proteins

Exemplary variable regions containing CD131-binding proteins have been previously described in WO 2017/088028 and/or are described in Table 1.

TABLE 1
Sequences of exemplary CD131-binding proteins

| | | V_{L} amino acid SEQ | V_{H} amino acid SEQ |
|---|---|---|---|
| | Antibody name | ID NO. | ID NO. |
| 1 | 9A2 | 5 | 20 |
| 2 | 9A2-VR1 | 6 | 20 |
| 3 | 9A2-VR2 | 7 | 20 |
| 4 | 9A2-VR3 | 8 | 20 |
| 5 | 9A2-VR4 | 9 | 20 |
| 6 | 9A2-VR5 | 10 | 20 |
| 7 | 9A2-VR6 | 11 | 20 |
| 8 | 9A2-VR8 | 12 | 20 |
| 9 | 9A2-VR9 | 13 | 20 |
| 10 | 9A2-VR11 | 14 | 20 |
| 11 | 9A2-VR12 | 15 | 20 |
| 12 | 9A2-VR13 | 16 | 20 |
| 13 | 9A2-VR14 | 17 | 20 |
| 14 | 9A2-VR16 | 18 | 20 |
| 15 | 9A2-VR19 | 19 | 20 |
| 16 | 9A2-VR20 | 5 | 21 |
| 17 | 9A2-VR21 | 5 | 22 |
| 18 | 9A2-VR22 | 5 | 23 |
| 19 | 9A2-VR23 | 5 | 24 |
| 20 | 9A2-VR24 | 5 | 25 |
| 21 | 9A2-VR26 | 5 | 26 |
| 22 | 9A2-VR27 | 5 | 27 |
| 23 | 9A2-VR28 | 5 | 28 |
| 24 | 9A2-VR31 | 5 | 29 |
| 25 | 9A2-VR32 | 5 | 30 |
| 26 | 9A2-VR33 | 5 | 31 |
| 27 | 9A2-VR34 | 5 | 32 |
| 28 | 9A2-VR35 | 5 | 33 |
| 29 | 9A2-VR36 | 5 | 34 |
| 30 | 9A2-VR37 | 5 | 35 |
| 31 | 9A2-VR38 | 5 | 36 |
| 32 | 9A2-VR39 | 5 | 37 |
| 33 | 9A2-VR40 | 5 | 38 |
| 34 | 9A2-VR41 | 5 | 39 |
| 35 | 9A2-VR42 | 5 | 40 |
| 36 | 9A2-VR43 | 5 | 41 |
| 37 | 9A2-VR44 | 5 | 42 |
| 38 | 9A2-VR45 | 5 | 43 |
| 39 | 9A2-VR46 | 5 | 44 |
| 40 | 9A2-VR47 | 5 | 45 |
| 41 | 9A2-VR48 | 5 | 46 |
| 42 | 9A2-VR49 | 5 | 47 |
| 43 | 9A2-VR50 | 5 | 48 |
| 44 | 9A2-VR24.04 | 5 | 49 |
| 45 | 9A2-VR24.07 | 5 | 50 |
| 46 | 9A2-VR24.10 | 5 | 51 |
| 47 | 9A2-VR24.12 | 5 | 52 |
| 48 | 9A2-VR24.19 | 5 | 53 |
| 49 | 9A2-VR24.24 | 5 | 54 |
| 50 | 9A2-VR24.76 | 5 | 55 |
| 51 | 9A2-VR24.78 | 5 | 56 |
| 52 | 9A2-VR24.81 | 5 | 57 |
| 53 | 9A2-VR24.82 | 5 | 58 |
| 54 | 9A2-VR24.84 | 5 | 59 |
| 55 | 9A2-VR24.87 | 5 | 60 |
| 56 | 9A2-VR24.91 | 5 | 61 |
| 57 | 9A2-VR24.93 | 5 | 62 |
| 58 | 9A2-VR24.27 | 5 | 63 |
| 59 | 9A2-VR24.29 | 5 | 64 |
| 60 | 9A2-VR24.30 | 5 | 65 |
| 61 | 9A2-VR24.33 | 5 | 66 |
| 62 | 9A2-VR24.44 | 5 | 67 |
| 63 | 9A2-VR24.97 | 5 | 68 |
| 64 | 9A2-VR24.98 | 5 | 69 |
| 65 | 9A2-VR24.102 | 5 | 70 |
| 66 | 9A2-VR24.107 | 5 | 71 |
| 67 | 9A2-VR24.110 | 5 | 72 |
| 68 | 9A2-VR24.111 | 5 | 73 |
| 69 | 9A2-VR24.55 | 5 | 74 |
| 70 | 9A2-VR24.56 | 5 | 75 |
| 71 | 9A2-VR24.57 | 5 | 76 |
| 72 | 9A2-VR24.122 | 5 | 77 |
| 73 | 9A2-VR24.124 | 5 | 78 |
| 74 | 9A2-VR24.131 | 5 | 79 |
| 75 | 9A2-VR39.01 | 5 | 80 |
| 76 | 9A2-VR39.02 | 5 | 81 |
| 77 | 9A2-VR39.04 | 5 | 82 |
| 78 | 9A2-VR39.05 | 5 | 83 |
| 79 | 9A2-VR39.06 | 5 | 84 |
| 80 | 9A2-VR39.11 | 5 | 85 |
| 81 | 9A2-VR39.12 | 5 | 86 |
| 82 | 9A2-VR39.16 | 5 | 87 |
| 83 | 9A2-VR39.17 | 5 | 88 |
| 84 | 9A2-VR39.18 | 5 | 89 |
| 85 | 9A2-VR39.19 | 5 | 90 |
| 86 | 9A2-VR39.21 | 5 | 91 |
| 87 | 9A2-VR39.22 | 5 | 92 |
| 88 | 9A2-VR39.23 | 5 | 93 |
| 89 | 9A2-VR39.24 | 5 | 94 |
| 90 | 9A2-VR39.97 | 5 | 95 |
| 91 | 9A2-VR39.98 | 5 | 96 |
| 92 | 9A2-VR39.102 | 5 | 97 |
| 93 | 9A2-VR39.103 | 5 | 98 |
| 94 | 9A2-VR39.105 | 5 | 99 |
| 95 | 9A2-VR39.109 | 5 | 100 |
| 96 | 9A2-VR39.110 | 5 | 101 |
| 97 | 9A2-VR39.111 | 5 | 102 |
| 98 | 9A2-VR39.112 | 5 | 103 |
| 99 | 9A2-VR39.116 | 5 | 104 |
| 100 | 9A2-VR39.27 | 5 | 105 |
| 101 | 9A2-VR39.28 | 5 | 106 |
| 102 | 9A2-VR39.46 | 5 | 107 |
| 103 | 9A2-VR39.122 | 5 | 108 |
| 104 | 9A2-VR39.139 | 5 | 109 |
| 105 | 9A2-VR39.140 | 5 | 110 |
| 106 | 9A2-VR39.148 | 5 | 111 |
| 107 | 9A2-VR39.162 | 5 | 112 |
| 108 | 9A2-VR39.77 | 5 | 113 |
| 109 | 9A2-VR39.93 | 5 | 114 |
| 110 | 9A2-VR39.174 | 5 | 115 |
| 111 | 9A2-VR39.177 | 5 | 116 |

### Protein Production

In one example, a protein described herein according to any example is produced by culturing a hybridoma under conditions sufficient to produce the protein, e.g., as described herein and/or as is known in the art.

### Recombinant Expression

In another example, a protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMl-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO1999/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Nucleic Acid Compounds that Bind to CD131

In one example, the compound that binds to CD131 is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., CD131. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to CD131) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. Nos. 5,270,163; and/or 5,475,096.

### Assaying Activity of a Compound

### Binding to CD131

Methods for assessing binding of a candidate compound to a protein (e.g., CD131) are known in the art, e.g., as described in Scopes (In: Protein purification: principles and practice, Third Edition, Springer Verlag, 1994). Such a method generally involves labeling the protein and contacting it with immobilized compound. Following washing to remove non-specific bound protein, the amount of label and, as a consequence, bound protein is detected. Of course, the protein can be immobilized and the compound labeled. Panning-type assays can also be used. Alternatively, or additionally, surface plasmon resonance assays can be used. The level of binding can also be conveniently determined using a biosensor.

Optionally, the dissociation constant (Kd) of a compound for CD131 or an epitope thereof is determined. The “Kd” or “Kd value” for a compound that binds to CD131 is in one example measured by a radiolabeled or fluorescently-labeled CD131 binding assay. This assay equilibrates the compound with a minimal concentration of labeled CD131 in the presence of a titration series of unlabeled CD131. Following washing to remove unbound CD131, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized CD131 or a region thereof.

### Epitope Mapping

In another example, the epitope bound by a protein described herein is mapped. Epitope mapping methods will be apparent to the skilled artisan. For example, a series of overlapping peptides spanning the CD131 sequence or a region thereof comprising an epitope of interest, e.g., peptides comprising 10-15 amino acids are produced. The protein is then contacted to each peptide and the peptide(s) to which it binds determined. This permits determination of peptide(s) comprising the epitope to which the protein binds. If multiple non-contiguous peptides are bound by the protein, the protein may bind a conformational epitope.

Alternatively, or in addition, amino acid residues within CD131 are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding CD131 or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in CD131 or a region thereof to deutrons and binding the resulting protein to an immobilized protein of the present disclosure. The deutrons are then converted back to hydrogen, the CD131 or region thereof isolated, digested with enzymes and analyzed, e.g., using mass spectrometry to identify those regions comprising deutrons, which would have been protected from conversion to hydrogen by the binding of a protein described herein.

Alternatively, the epitope to which the protein binds can be determined by X-ray crystallography. For example, a complex between the protein and CD131 is formed and then crystalized. The resulting crystals are then subjected to x-ray diffraction analysis to determine the atomic co-ordinates of the amino acids in the complex. The epitope comprises the amino acids in CD131 that are in contact with the protein, according to the atomic co-ordinates determined from the x-ray diffraction.

### Determining Competitive Binding

Assays for determining a protein that competitively inhibits binding of antibody described herein will be apparent to the skilled artisan. For example, exemplary antibody CSL311 is conjugated to a detectable label, e.g., a fluorescent label or a radioactive label. The labeled antibody and the test protein are then mixed and contacted with CD131 or a region thereof (e.g., a polypeptide comprising SEQ ID NO: 1) or a cell expressing same. The level of labeled CSL311 is then determined and compared to the level determined when the labeled antibody is contacted with the CD131, region or cells in the absence of the protein. If the level of labeled CSL311 is reduced in the presence of the test protein compared to the absence of the protein, the protein is considered to competitively inhibit binding of CSL311 to CD131.

Optionally, the test protein is conjugated to different label to CSL311. This alternate labeling permits detection of the level of binding of the test protein to CD131 or the region thereof or the cell.

In another example, the protein is permitted to bind to CD131 or a region thereof (e.g., a polypeptide comprising SEQ ID NO: 1) or a cell expressing same prior to contacting the CD131, region or cell with CSL311. A reduction in the amount of bound CSL311 in the presence of the protein compared to in the absence of the protein indicates that the protein competitively inhibits CSL311 binding to CD131. A reciprocal assay can also be performed using labeled protein and first allowing CSL311 to bind to CD131. In this case, a reduced amount of labeled protein bound to CD131 in the presence CSL311 compared to in the absence of CSL311 indicates that the protein competitively inhibits binding of CSL311 to CD131.

Any of the foregoing assays can be performed with a mutant form of CD131 and/or SEQ ID NO: 1 and/or a ligand binding region of CD131 to which CSL311 binds, e.g., as described herein.

### Determining Neutralization

In one example, the compound that binds to CD131 reduces or prevents binding of IL-3, IL-5 and/or GM-CSF to a receptor comprising CD131 (e.g., an IL-3R, an IL-5R and/or a GM-CSF-R, respectively). These assays can be performed as a competitive binding assay using labeled IL-3/Il-5/GM-CSF and/or labeled compound. For example, cells expressing the relevant receptor is contacted with IL-3/Il-5/GM-CSF in the presence or absence of a CD131-binding compound and the amount of bound label detected. A reduction in the amount of bound label in the presence of the CD131-binding compound compared to in the absence of the compound indicates that the compound reduces or prevents binding of IL-3/Il-5/GM-CSF to a receptor comprising CD131. By testing multiple concentrations of the compound an IC₅₀ is determined, i.e., a concentration of the compound that reduces the amount of IL-3/Il-5/GM-CSF that binds to a receptor comprising CD131, or an EC₅₀ can be determined, i.e., a concentration of the protein that achieves 50% of the maximum inhibition of binding of IL-3/Il-5/GM-CSF to CD131 achieved by the compound.

In a further example, the CD131-binding compound reduces or prevents IL-3/Il-5/GM-CSF-mediated proliferation of leukemic cell line TF-1. For example, TF-1 cells are cultured without IL-3/Il-5/GM-CSF for a time sufficient for them to stop proliferating (e.g., 24-48 hours). Cells are then cultured in the presence of IL-3/Il-5/GM-CSF and various concentrations of the CD131-binding compound. Control cells are not contacted with the compound (positive control) or IL-3/Il-5/GM-CSF (negative control). Cell proliferation is then assessed using a standard technique, e.g., ³H-thymidine incorporation. A CD131-binding compound that reduces or prevents cell proliferation in the presence of IL-3 to a level less than the positive control is considered to neutralize IL-3 signaling. By testing multiple concentrations of the CD131-binding compound, an IC₅₀ is determined.

In a further example, a CD131-binding compound of the disclosure affects an immune cell. For example, the CD131-binding compound reduces or inhibits activation of isolated human neutrophils by GM-CSF as determined by reducing or inhibiting GM-CSF-induced increase in neutrophil cell size. For example, neutrophils (e.g., about 1×10⁵ cells) are cultured in the presence of the CD-131-binding protein and GM-CSF for a suitable time (e.g., about 24 hours). Cells are then fixed (e.g., with formaldehyde) and analyzed for forward scatter using flow cytometry.

In a further example, the CD131-binding compound reduces or prevents activation of human peripheral blood eosinophils by IL-5 as determined by assessing change in forward scatter assessed by flow cytometry. For example, eosinophils (e.g., about 1×10⁵ cells) are cultured in the presence of a CD131-binding compound and IL-5 for a suitable time (e.g., about 24 hours). Cells are then fixed (e.g., in formaldehyde) and assessed for change in forward scatter, e.g., using flow cytometry.

In a further example, a CD131-binding compound of the disclosure reduces or prevents survival of human peripheral blood eosinophils. For example, the compound prevents reduces or prevents survival of eosinophils in the presence of IL-5 and/or GM-CSF and/or IL-3. For example, eosinophils (e.g., about 1×10⁴ cells) are cultured in the presence of a CD131-binding compound and IL-5 and/or GM-CSF and/or IL-3 for a suitable time (e.g., about 5 days) and cell numbers assessed using a standard method (e.g., a ViaLight Plus Kit from Lonza).

In a further example, a CD131-binding compound reduces accumulation of or survival of or induces death of immune cells (e.g., eosinophils) from bronchoalveolar (BAL) fluid or lung tissue from a subject suffering from an eosinophil-associated COPD. For example, the immune cells are cultured in the presence of IL-3 and/or IL-5 and/or GM-CSF and the protein or antibody. Cell death is then assessed using standard methods, e.g., by detecting Annexin-V expression, e.g., using fluorescence activated cell sorting.

Other methods for assessing neutralization of GM-CSF, IL-5 or IL-3 signaling are contemplated by the present disclosure.

### Therapeutic Efficacy

The therapeutic efficacy of a compound that binds to CD131 can be assessed by comparing the degree of severity of the disease or symptoms in subjects administered with the compound relative to subjects not administered the compound. Alternatively, or additionally, therapeutic efficacy of candidate compounds can be assessed in an animal model. Suitable assays for assessing therapeutic efficacy are described.

In one example, the efficacy of a protein to treat a condition is assessed using an in vivo assay.

In one example, the CD131-binding compound is administered to a non-human animal (e.g., a non-human primate) and the number/level of immune cells, e.g., eosinophils, in circulation or in a tissue or other sample (e.g., skin tissue at the site of inflammation) is assessed. A CD131-binding compound that reduces the number/level of immune cells compared to prior to administration and/or in a control mammal to which the protein has not been administered is considered suitable for treating the disease or condition.

In one example, a CD131-binding compound is tested in a COPD model of acute cigarette smoke exposure. In such models, a non-human mammal (e.g., a rodent, such as a mouse) is exposed to cigarette smoke. In one example of such models, there is no evidence of airway wall remodelling. For example, there is no evidence of an increase in collagen deposition and/or mucous hyperplasia. Following exposure, the mammal is administered a CD131-binding protein and the level of lung inflammation and/or the number of neutrophils in the lung is assessed or estimated using standard techniques. A CD131-binding protein that reduces lung inflammation and/or the number of neutrophils is considered useful for treating COPD.

In one example, a CD131-binding protein is tested in a model of eosinophil-associated COPD/asthma overlap (ACO). In such models, a non-human mammal (e.g., a rodent, such as a mouse) is sensitised, and subsequently challenged, with a house dust mite extract (HDM) and porcine pancreatic elastase (PPE). Following exposure, the mammal is administered a CD131-binding protein and the level of lung inflammation and/or the number of eosinophils and/or neutrophils in the lung is assessed or estimated using standard techniques. A CD131-binding protein that reduces lung inflammation and/or the number of eosinophils and/or neutrophils is considered useful for treating eosinophil-associated COPD/asthma overlap (ACO).

In another example, the CD131-binding protein is administered to a non-human animal (e.g., a non-human primate) and the number/level of immune cells, e.g., eosinophils, in circulation or in a tissue or other sample (e.g., BAL fluid) is assessed. A CD131-binding protein that reduces the number/level of immune cells compared to prior to administration and/or in a control mammal to which the protein has not been administered is considered suitable for treating the disease or condition.

In some examples, assessing the therapeutic efficacy of a compound comprises detecting and/or quantifying the level of expression of a biomarker in the subject. Suitable biomarkers for assessing efficacy of treating eosinophil associated COPD include IL-1α, IL-17a, IL-23a, C-X-C Motif Chemokine Ligand 1 (CXCL1), CXCL2, C-C Motif Chemokine Ligand 2 (CCL2), matrix metalloproteinase 12 (MMP-12), Found in Inflammatory Zone 1 (Fizz1) and GM-CSF.

Detecting and/or quantifying biomarkers can be performed by any method known in the art. For instance, in one example, the levels of biomarkers are assessed using mass spectrometry. The mass spectrometry may be performed in conjunction with ultra-performance liquid chromatography (UPLC), high-performance liquid chromatography (HPLC), gas chromatography (GC), gas chromatography/mass spectroscopy (GC/MS), and UPLC, for example. Other methods of assessing levels of biomarkers include biological methods, such as but not limited to ELISA assays, Western Blot and multiplexed immunoassays etc. Other techniques may include using quantitative arrays, PCR, Northern Blot analysis. To determine levels of components or factors, it is not necessary that an entire component, e.g., a full length protein or an entire RNA transcript, be present or fully sequenced. In other words, determining levels of, for example, a fragment of protein being analyzed may be sufficient to conclude or assess that the level of the biomarker being analyzed is increased or decreased. Similarly, if, for example, arrays or blots are used to determine component levels, the presence/absence/strength of a detectable signal may be sufficient to assess levels of biomarkers.

To assess levels of biomarkers, a sample may be taken from the subject. The sample may or may not processed prior assaying levels of the components of the biomarker profile. For example, whole blood may be taken from an individual and the blood sample may be processed, e.g., centrifuged, to isolate plasma or serum from the blood. The sample may or may not be stored, e.g., frozen, prior to processing or analysis.

Biological samples that may be tested in a method of the invention include whole blood, blood serum, plasma, tracheal aspirate, BALF, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

### Compositions

In one example, a CD131-binding compound as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, intrapolyp and intracranial injection or infusion techniques.

Methods for preparing a CD131-binding compound into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of a CD131-binding compound dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a CD131-binding compound of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a CD131-binding compound of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver a CD131-binding compound of the present disclosure.

WO2002/080967 describes compositions and methods for administering aerosolized compositions comprising antibodies for the treatment of respiratory conditions, which are also suitable for administration of compounds in accordance with the methods of the present disclosure.

### Combination Therapies

In one example, a CD131-binding compound of the present disclosure is administered in combination with another compound or therapy useful for treating a condition described herein, either as combined or additional treatment steps or as additional components of a therapeutic formulation.

For example, the other compound is a bronchodilator. In one example, the bronchodilator is a short-acting bronchodilator such as albuterol (e.g., ProAir® HFA, Ventolin® HFA), ipratropium (e.g., Atrovent® HFA) and levalbuterol (e.g., Xopenex®). In another example, the bronchodilator is a long-acting bronchodilator such as aclidinium (e.g., Tudorza® Pressair®), arformoterol (e.g., Brovana®), formoterol (e.g., Perforomist®), indacaterol (e.g., Arcapta® Neoinhaler®), tiotropium (e.g., Spiriva®), salmeterol (e.g., Serevent®) and umeclidinium (e.g., Incruse® Ellipta®).

In one example, the other compound is a combination of more than one bronchodilators. Exemplary combination bronchodilators include aclidinium and formoterol (e.g., Duaklir® Pressair®), albuterol and ipratropium (e.g., Combivent® Respimat®), formoterol and glycopyrrolate (e.g., Bevespi® Aerosphere®), glycopyrrolate and indacaterol (e.g., Utibron®), olodaterol and tiotropium (e.g., Stiolto® Respimat®) and umeclidinium and vilanterol (e.g., Anoro® Ellipta®).

In another example, the other compound is a steroid, for example a corticosteroid. Exemplary corticosteroids include prednisone and/or prednisolone.

In one example, the steroid is an inhaled steroid. Exemplary inhaled steroids (i.e., corticosteroids) include fluticasone (e.g., Flovent® HFA) and budesonide (e.g., Pulmicort Flexhaler®).

In one example, the other compound is a combination bronchodilator and corticosteroid. Exemplary combinations include fluticasone and vilanterol (e.g., Breo® Ellipta®), fluticasone, umeclidinium and vilanterol (e.g., Trelegy® Ellipta®), formoterol and budesonide (e.g., Symbicort®) and salmeterol and fluticasone (e.g., Advair® HFA, AirDuo® Digihaler®).

In one example, the other compound is an antibody therapy. Exemplary antibody therapies include mepolizumab (e.g., Nucala®) and benralizumab (e.g., Fasenra®).

In one example, the other therapy is oxygen therapy.

The present disclosure also provides a method for reducing the dosage of the other compound or therapy required to treat a subject with eosinophil-related COPD, the method comprising co-administering a CD131-binding compound described herein and the other compound or therapy, wherein the other compound or therapy is administered at a lower dose than if it were administered alone or in the absence of the CD131-binding compound. The CD131-binding compound and the other compound or therapy need not be administered at the same time, only in such a manner that they have an overlapping effect on the subject (e.g., are both active within the subject at the same time).

In one example, the CD131-binding compound is administered simultaneously with the other compound or therapy. In one example, the CD131-binding compound is administered before the other compound or therapy. In one example, the CD131-binding compound is administered after the other compound or therapy.

### Dosages and Timing of Administration

Suitable dosages of a CD131-binding compound of the present disclosure will vary depending on the specific CD131-binding compound, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically or prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present disclosure comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of protein(s), rather the present disclosure encompasses any amount of the CD131-binding compound(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific C131-binding protein(s) administered and/or the particular subject and/or the type or severity or level of condition and/or predisposition (genetic or otherwise) to the condition. Accordingly, this term is not to be construed to limit the present disclosure to a specific quantity, e.g., weight or amount of CD131-binding compound(s), rather the present disclosure encompasses any amount of the C131-binding protein(s) sufficient to achieve the stated result in a subject.

For in vivo administration of the CD131-binding compound described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the CD131-binding compound is administered at an initial (or loading) dose of between about 1 mg/kg to about 30 mg/kg, such as from about 1 mg/kg to about 10 mg/kg, or about 1 mg/kg or about 2 mg/kg or 5 mg/kg. The CD131-binding compound can then be administered at a lower maintenance dose of between about 0.01 mg/kg to about 2 mg/kg, such as from about 0.05 mg/kg to about 1 mg/kg, for example, from about 0.1 mg/kg to about 1 mg/kg, such as about 0.1 mg/kg or 0.5 mg/kg or 1 mg/kg. The maintenance doses may be administered every 7-30 days, such as, every 10-15 days, for example, every 10 or 11 or 12 or 13 or 14 or 15 days.

In some examples, the CD131-binding compound is administered at a dose of between about 0.01 mg/kg to about 50 mg/kg, such as between about 0.05 mg/kg to about 30 mg/kg, for example, between about 0.1 mg/kg to about 20 mg/kg, for example, between about 0.1 mg/kg to about 10 mg/kg, such as between about 0.1 mg/kg to about 2 mg/kg. For example, the CD131-binding compound is administered at a dose of between about 0.01 mg/kg to about 5 mg/kg, such as from about 0.1 mg/kg to about 2 mg/kg, such as about 0.2 mg/kg or 0.3 mg/kg or 0.5 mg/kg or 1 mg/kg or 1.5 mg/kg (e.g., without a higher loading dose or a lower maintenance dose). In some examples, numerous doses are administered, e.g., every 7-30 days, such as, every 10-22 days, for example, every 10-15 days, for example, every 10 or 11 or 12 or 13 or 14 or 15 or 16 or 17 or 18 or 19 or 20 or 21 or 22 days. For example, the CD131-binding compound is administered every 7 days or every 14 days or every 21 days.

In some examples, at the time of commencing therapy, the subject is administered the CD131-binding compound on no more than 7 consecutive days or 6 consecutive days or 5 consecutive days or 4 consecutive days.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of a CD131-binding compound according to the methods of the present disclosure can be continuous or intermittent, depending, for example, on the recipient's physiological condition, whether the purpose of the administration is therapeutic or prophylactic, and other factors known to skilled practitioners. The administration of a CD131-binding compound may be essentially continuous over a preselected period of time or may be in a series of spaced doses, e.g., either during or after development of a condition.

### Kits

Another example of the disclosure provides kits containing compounds useful for the treatment or prevention of an eosinophil-associated COPD as described above.

In one example, the kit comprises (a) a container comprising a compound that binds to CD131 as described herein, optionally in a pharmaceutically acceptable carrier or diluent; and (b) a package insert with instructions for treating, preventing, or reducing an effect of an eosinophil-associated COPD in a subject.

In one example, the kit comprises (a) a container comprising a compound that binds to CD131 as described herein, optionally in a pharmaceutically acceptable carrier or diluent; and (b) a package insert with instructions for treating, preventing, or reducing eosinophilia associated with a smoking-related COPD in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating or preventing the eosinophil-associated COPD or smoking-related COPD and may have a sterile access port (for example, the container may be an intravenous solution bag or a vial having a stopper pierceable by a hypodermic injection needle). At least one active agent in the composition is the compound that binds to CD131. The label or package insert indicates that the composition is administered to a subject eligible for treatment, e.g., one having or at risk of developing an eosinophil-associated COPD, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Materials and Methods

### Animal Experimental Protocols.

All animal experiments were approved at RMIT University in accordance with the National Health and Medical Research Council of Australia (NHMRC) and ARRIVE guidelines. Humanised transgenic mice where the endogenous mouse βc and β_{IL-3} receptor have been knocked out and replaced with the human (h)βc receptor were used.

Twelve- to fourteen-week-old male hβc transgenic mice were housed in sterile microisolators at 20° C. on 12-hour day-night cycles and provided a standard chow diet for Laboratory Rats and Mice (Specialty Feeds, WA, Australia) with water available ad-libitum. Mice were exposed to cigarette smoke as previously described (see e.g., Vlahos R et al., Am J Respir Crit Care Med. 2010; 182(1):34-40) for 11 consecutive days. Briefly, mice were placed in an 18-litre Perspex container and exposed to a total of 9 cigarettes per day over 3×15-minute exposure sessions. This method achieves a total suspended particulate mass concentration of approximately 420 mg/m³ in the cigarette smoke container. Sham mice were placed in a separate identical Perspex container and were exposed to room air. Commercially available filter-tipped cigarettes (manufactured by Philip Morris, Australia) of the following composition were used: 16 mg or less of tar, 1.2 mg or less of nicotine, and 15 mg or less of CO.

CSL311, a human monoclonal antibody that blocks βc cytokine binding and signalling and matching isotype control antibody were used. Four doses of CSL311 antibody (50 mg/kg, CSL Limited, Australia) or isotype control were administered via intravenous injection on day 1, day 4, day 8 and day 11 prior to the cigarette smoke exposure session.

### Bronchoalveolar Lavage, Cytospot and Lung Tissue Collection

On day 12, hTgβc mice were euthanised by pentobarbital overdosing (i.p., 240 mg/kg) via intraperitoneal injection. Blood was collected into MiniCollect® K3EDTA tubes (Sardtest, Australia) via right ventricular puncture and subjected to haematology analysis (Cell-Dyn Emerald, Abbott Laboratories, US). Bronchoalveolar lavage (BAL) was performed by flushing using a 21-gauge cannula resulting in the collection of fluid (BALF) and the total number of viable cells were calculated with a haemocytometer as previously described (see e.g., Anthony D, et al., FASEB J. 2014; 28(9):3867-77). A fraction of BAL cells was used to generate cytospin slides and stained with a Hemacolor® Rapid Staining Kit (Sigma-Aldrich, US) for differential cell counting. The remaining BAL cells were centrifuged, and BAL cell pallets and cell-free BAL fluid was collected and stored at −80° C. for further analysis. The remaining lobes were snap-frozen in liquid nitrogen and subsequently stored at −80° C. Lungs were harvested following perfusion with PBS and the left lung was fixed in 10% neutral-buffered formalin for histology.

### Lung Histology

The fixed left lung lobe was paraffin-embedded and sliced into 4 μm sections. Slides were stained with H&E and images were captured using a whole slide scanner (VS120 Slidescanner, Olympus, Japan) for lung injury analysis. Lung injury was scored based on the degree of inflammatory cell infiltration, epithelial/endothelial destruction, and alveolar septal thickening using an aggregate scoring scale as previously described (see e.g., Wang H, et al., Br J Pharmacol. 2021; 178(8):1869-85). Scoring was performed in a blinded manner and a combined score of three regions (perivascular, peribronchial and parenchyma) was used for each section.

### Lung Injury and Oedema Assays

Lung injury was determined by measuring levels of lactate dehydrogenase (LDH) in the BALF using the Pierce™ LDH Cytotoxicity Assay Kit (Thermofisher Scientific, US) as per manufacturer's instructions. Lung oedema was determined by measuring levels of serum albumin in the BALF using the Bromocresol Green Albumin Assay kit (Sigma-Aldrich, US) as per manufacturer's instructions. Gelatinase activity in the BALF was measured using the EnzChek™ Gelatinase Assay Kit (Thermofisher Scientific, US). Cell-free dsDNA content in the BALF was determined using the Quant-iT™ PicoGreen™ dsDNA Assay Kit (Thermofisher Scientific, US).

### Flow Cytometry

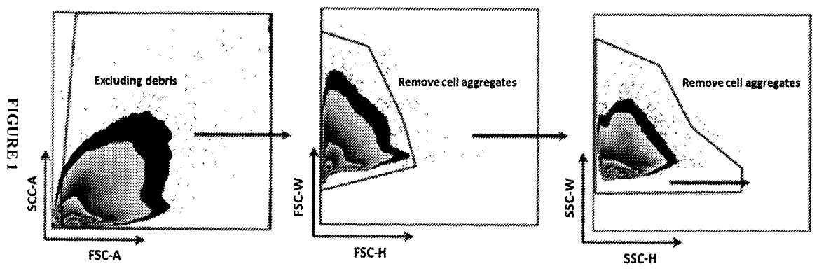

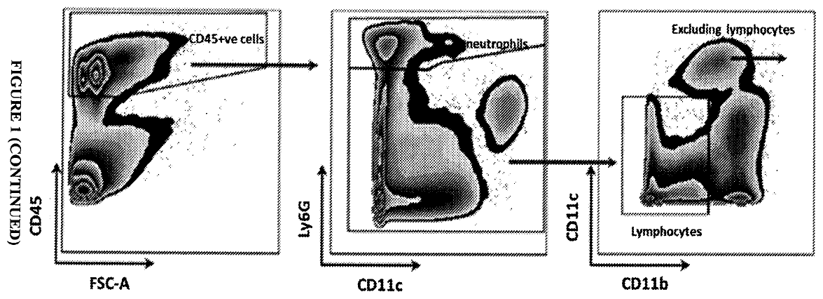

The right superior lobe was prepared for flow cytometry as previously described (see e.g., Wang et al., 2021). Briefly, the lung lobe was digested in Liberase™ (Sigma-Aldrich, US) to obtain a single cell suspension, which was then pelleted and treated with Ammonium-Chloride-Potassium (ACK) red blood cell lysis buffer. The remaining cells were stained with a panel of fluorescein-conjugated antibodies consisting: FITC-CD45, PE-Siglec F, APC-F4/80, eFluor 450-CD11b, PE/Cy7-CD11c, PerCp/eFluor710-Ly6G and analysed on a BD LSRFortessa™ X-20 (BD Biosciences, US). All antibodies were obtained from Thermofisher Scientific, US. Gating strategies used to identify neutrophil, eosinophils and macrophage subsets are shown in FIG. 1.

RNA Extraction, cDNA Conversion and Real-Time Quantitative PCR

Fresh-frozen lung tissue was ground in liquid nitrogen with pestle and mortar. Total RNA was extracted from −30 mg crushed lung tissue using a RNeasy kit (Qiagen, Germany) then converted to cDNA (High-Capacity cDNA Reverse Transcription Kit, Thermofisher Scientific, US). Real-time quantitative PCR (RTqPCR) was then performed with a TaqMan® Fast Advanced Master Mix kit (Applied Biosystems, US) on the Quantstudio™ 7 PCR system (Applied Biosystems, US). Genes of interest were normalised against housekeeping gene GAPDH and expressed as fold change by performing the delta-delta Ct method.

### Statistical Analysis

All statistical analyses were performed with GraphPad Prism 9.0 and graphical data are presented as mean±SEM. Student's t-tests were performed where appropriate. Statistical significance is declared where p<0.05 and is indicated with an asterisk (*).

### Example 2: Cigarette Smoke Induces Systemic Inflammation and Lung Injury in hβcTg Mice

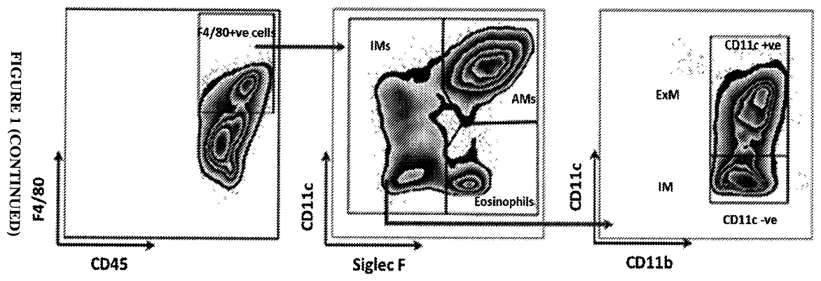

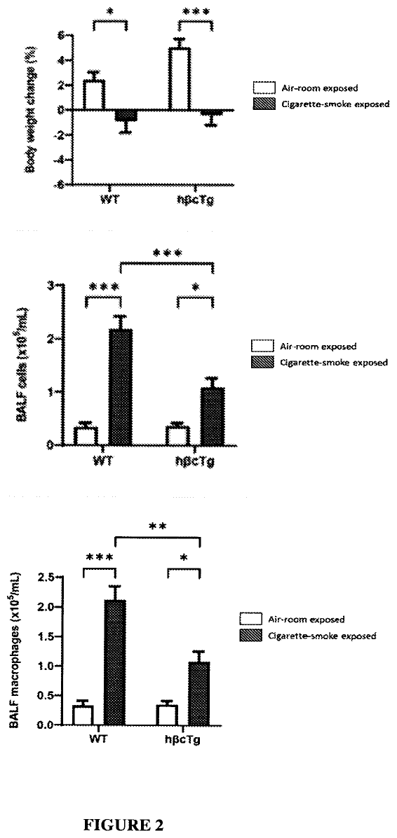

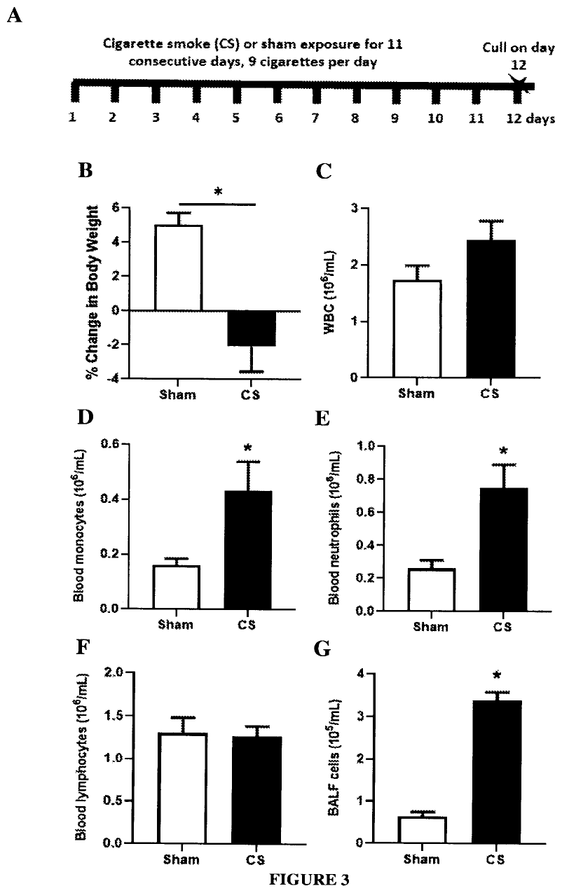

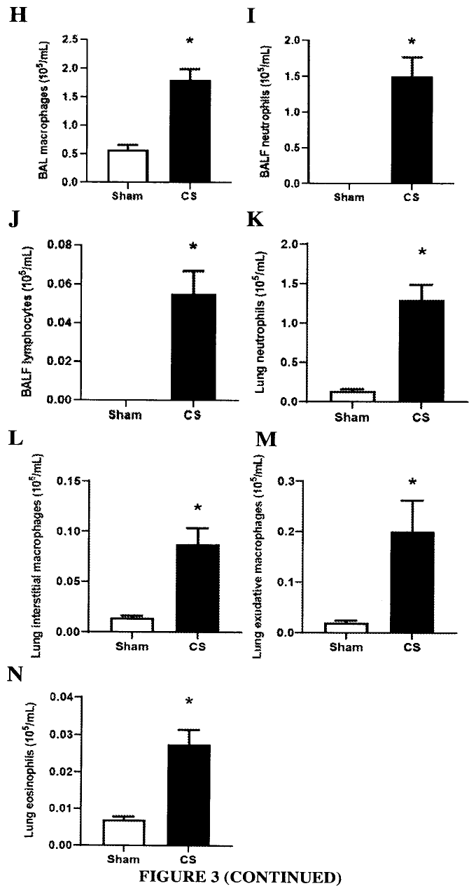

The cigarette smoke response in hβcTg mice was first compared with wildtype (WT) mice in an acute 4-day cigarette smoke exposure model (as previously described in Vlahos et al Am J Physiol Lung Cell Mol Physiol, 2006). hβcTg mice elicited a similar degree of body weight loss (FIG. 2A) and displayed a blunted BAL inflammatory profile compared to WT mice, the latter of which was mainly attributed to lower BAL macrophage numbers (FIGS. 2B and C). hβcTg mice were exposed to cigarette smoke (9 cigarettes/day) over 11 days as described above and as shown in FIG. 3A. Cigarette smoke exposure caused a significant 2% loss in body weight, whereas room air-exposed mice gained 4% body weight by the end of the protocol (FIG. 3B). Whilst the total white blood cell count was not significantly altered by cigarette smoke exposure (FIG. 3C), there was an approximately 2-fold increase in circulating neutrophils and monocytes, whereas lymphocyte numbers were not altered (FIG. 3D-F). Cigarette smoke exposure also caused significant BAL inflammation, determined by the increases in total BAL cells (FIG. 3G). Differential cell analysis demonstrated a 3-fold increase in BAL macrophage numbers, a large influx in BAL neutrophils and smaller increase of BAL lymphocytes in CS-exposed (FIG. 3H-J). Flow cytometry was employed to analyse myeloid cell populations in lung tissue. Like the BAL compartment, significant recruitment of inflammatory cells into the lungs was observed. There were approximately 3.5 times more neutrophils in the lungs of cigarette smoke-exposed mice compared to room air-exposed mice (FIG. 3K). Macrophages were further immunophenotyped, which revealed a significant increase in IMs (CD11b⁺CD11c⁻) and ExMs (CD11b⁺CD11c⁺) (FIG. 3L-M). Lung eosinophils were analysed, demonstrating an approximate 30% increase in number in cigarette smoke-exposed mice (FIG. 3N).

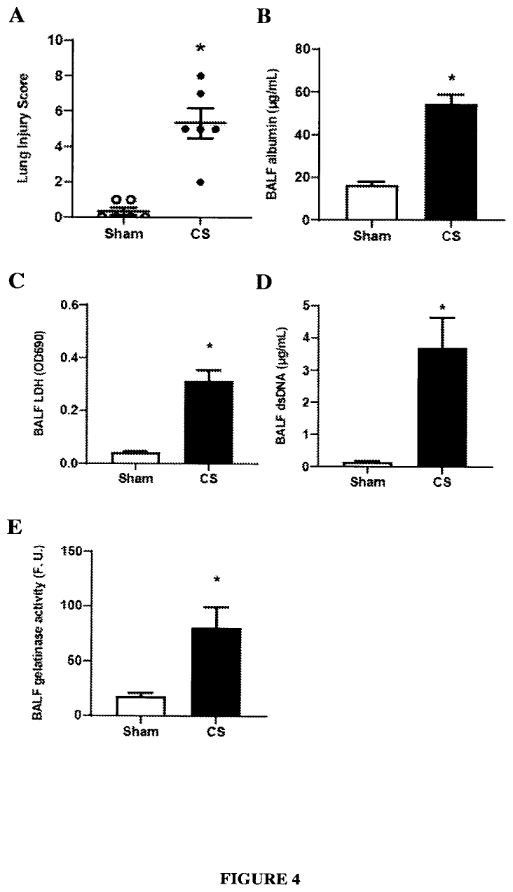

Cigarette smoke exposure resulted in alveolar, perivascular and peribronchiolar inflammation throughout the lung lobe sections. The degree of inflammation and injury was scored across entire lung lobe sections, where cigarette smoke-exposed lungs averaged a significantly higher injury score compared to sham mice (FIG. 4A). Serum albumin levels in BALF were increased in cigarette smoke-exposed mice, consistent with compromised alveolar-capillary barrier integrity leading to lung oedema (FIG. 4B) In addition, lactate dehydrogenase (LDH) levels were also increased by over 7-fold in the BALF of cigarette smoke-exposed mice, further indicating that cigarette smoke exposure induced lung epithelial/endothelial cell death and tissue damage (FIG. 4C). These changes were accompanied by a 4.5-fold increase in gelatinase activity and a 20-fold increase in cell-free dsDNA content in the BALF supernatant (FIG. 4D-E), which are markers of neutrophil degranulation, netosis and cell death.

### Example 3: Cigarette Smoke Increases Expression of Inflammatory Cytokines in Lung Tissue

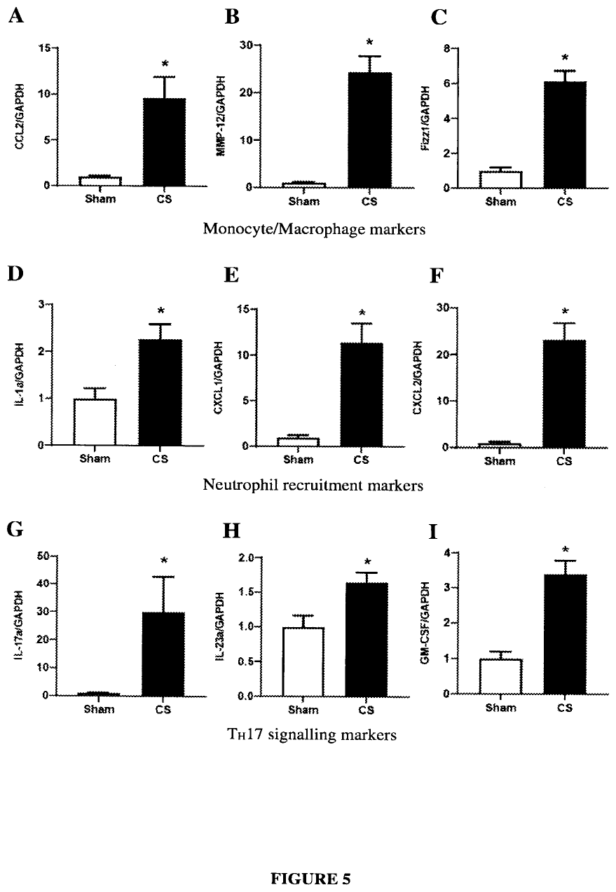

To further elucidate the inflammatory profile induced by cigarette smoke in the lung tissue of hβcTg mice, a panel of inflammatory markers were analysed using real-time quantitative PCR. The expression of the monocyte chemokine C-C motif chemokine ligand 2 (CCL2) was significantly increased in cigarette smoke-exposed mice (FIG. 5A). Furthermore, the M2 macrophage activation markers Fizz1 and macrophage elastase MMP-12 were also significantly increased (FIG. 5B-C), consistent with an increase in alternatively activated macrophages in the lungs of cigarette smoke-exposed mice. Cigarette smoke exposure also induced the expression of the inflammatory cytokine interleukin 1-alpha (IL-1α), which was 2-fold higher compared to sham mice. In addition, the neutrophil chemokines C-X-C motif ligand 1 and 2 (CXCL1, CXCL2) were increased more than 10-fold and 20-fold respectively (FIG. 5E-F), which is also consistent with the prominent neutrophil trafficking in to the lungs and BAL compartment. Since TH17 signalling can regulate inflammation in cigarette smoke-exposed mice, we measured IL-17a and IL-23a, which were significantly increased in cigarette smoke-exposed mice (FIG. 5G-H). TH17 cells have been identified as an important cellular source of CSF2 (GM-CSF), which was also increased in the lungs of CS-exposed mice (FIG. 5I). There was no difference in IL-5 lung expression and IL-3 was below our detection limit (not shown).

### Example 4: Cigarette Smoke Induced Inflammation and Lung Injury is Blocked by CSL311 Antibody Treatment

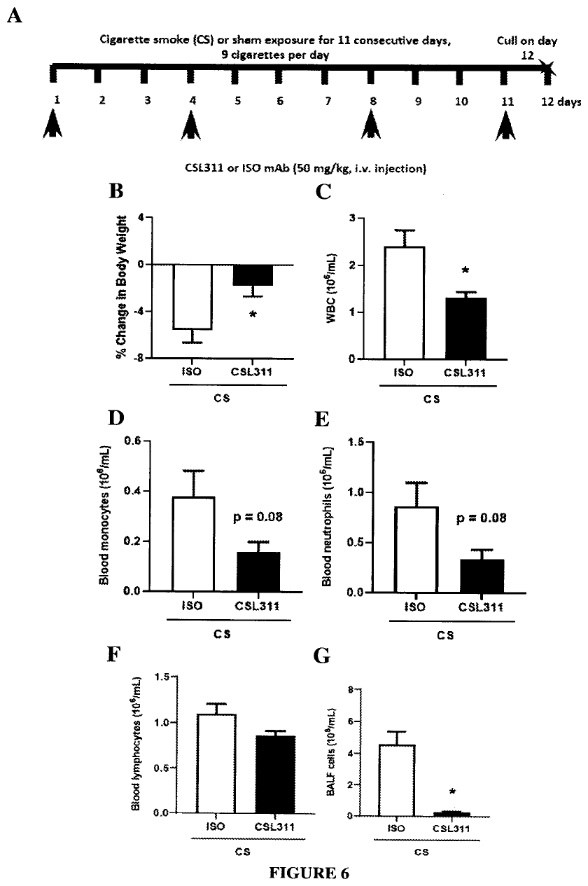

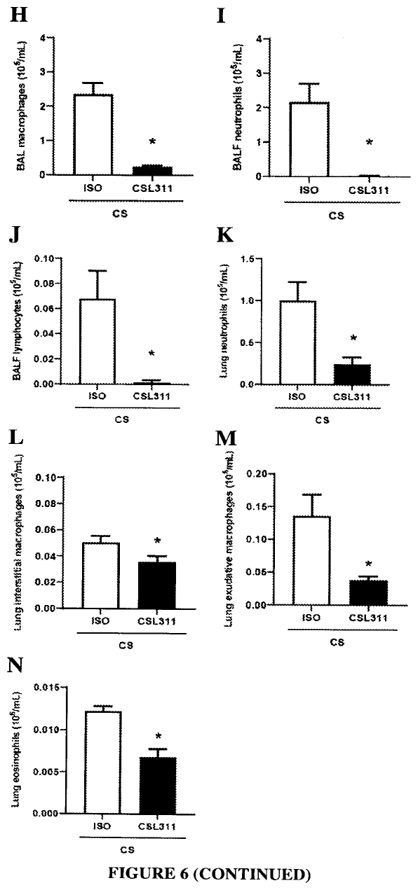

hβcTg mice exposed to cigarette smoke were treated with CSL311 or ISO antibody on Day 1, 4, 8 and 11 as shown in FIG. 6A, where CSL311 treatment reduced the percentage weight loss compared to isotype treated mice (FIG. 6B; 1.8 vs 5.6%). The total WBC was significantly decreased from 2.4×10⁶/mL (isotype control) to 1.3×10⁶/mL with CSL311 treatment (FIG. 6C). There was a trend towards reduced blood monocyte numbers (FIG. 6D; ISO; 3.8×10⁵/mL vs. CSL311; 1.6×10⁵/mL, p=0.08) in cigarette smoke-exposed mice. Likewise, there was a trend towards decreased blood neutrophil numbers (FIG. 6E; ISO; 8.6×10⁵/mL vs. CSL311; 3.4×10⁵/mL, p=0.08) in cigarette smoke-exposed mice. No difference in blood lymphocytes was observed (FIG. 6F). BAL inflammation was next evaluated where CSL311 treatment markedly reduced total BAL cells, BAL macrophage, BAL neutrophil and BAL lymphocyte numbers (FIG. 6G-J), essentially blocking immune cell infiltrate to sham/control numbers. Flow cytometry comparing cigarette smoke-exposed mice treated with CSL311 vs. ISO antibody demonstrated that CSL311 effectively reduced lung neutrophil numbers, lung IMs (CD11b⁺CD11c⁻), lung ExMs (CD11b⁺CD11c⁺), alveolar macrophages (AMs) and lung eosinophil numbers (FIG. 6K-N).

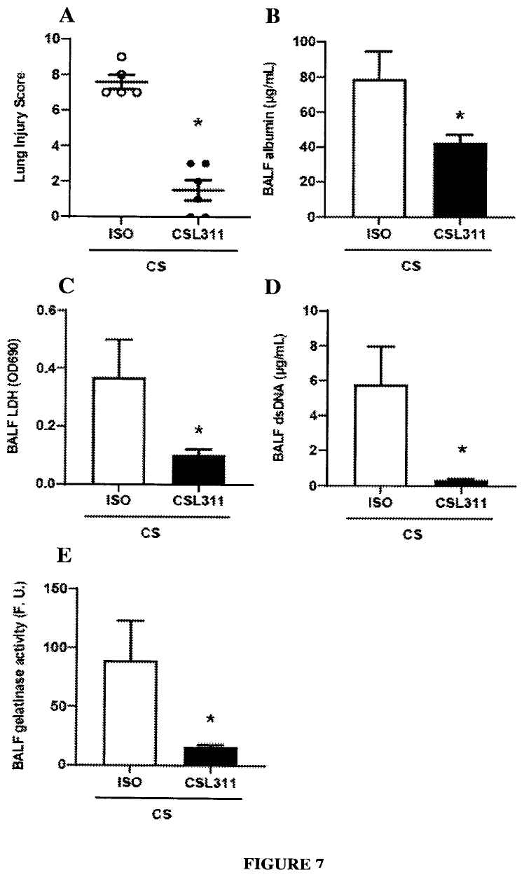

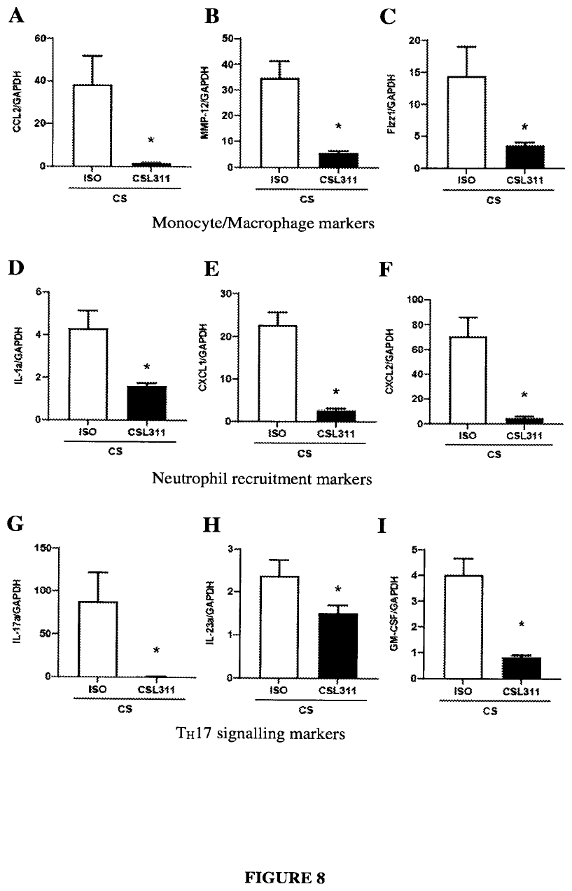

Examination of histological lung sections showed that cigarette smoke-exposed mice treated with isotype antibody exhibited high levels of alveolar, peribronchiolar and perivascular infiltration of immune cells, which was significantly lower in cigarette smoke-exposed mice treated with CSL311 (FIG. 7A). The reduction in lung injury was confirmed by significant decreases in BALF markers of lung oedema, where there was a 50% reduction in BALF albumin concentration in CSL311 treated mice compared to isotype treated mice (FIG. 7B). LDH levels were also significantly reduced by over 3-fold, consistent with reduced lung mucosal cell damage and death (FIG. 7C). Additionally, dsDNA and gelatinase levels were found to be significantly lower in the BALF of CSL311 treated mice compared to isotype control (FIG. 7D-E). hβc receptor antagonism with CSL311 also significantly decreased expression of the monocyte chemokine CCL2, as well as M2 macrophage markers MMP-12 and Fizz1 (FIG. 8A-C), which was consistent with the reduction in IM and ExM numbers in the lungs. Furthermore, CSL311 also reduced expression of IL-1α and the neutrophil chemokines CXCL1 and CXCL2 (FIG. 8D-F), consistent with their dramatic reduction in the BAL compartment. The TH17 marker IL-17a was almost completely suppressed (FIG. 8G) and there was a significant reduction in the expression of IL-23 and GM-CSF (FIG. 8H-I), suggesting that hβc receptor antagonism may deplete TH17 cells and reduce TH17 mediated inflammation.

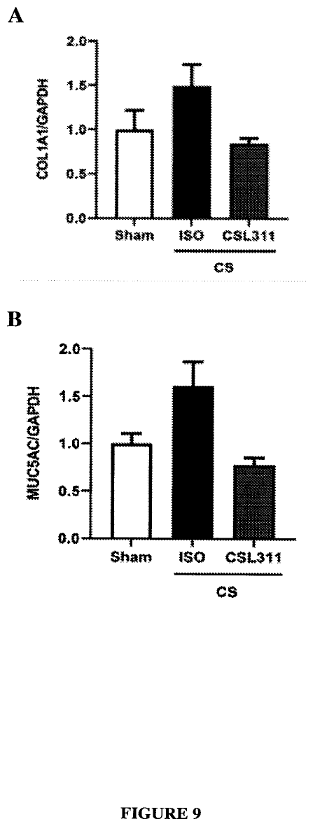

Masson's trichrome (MT) stained lung sections detected no difference in collagen deposition in CS-exposed mice, however there was a trend towards increased COLA1 expression in CS-exposed mice, which was reduced with CSL311 treatment (FIG. 9A). Likewise, no change was observed in AB-PAS stained lung sections in this acute model of CS-exposure, however MUC5AC expression trended to increase in CS-exposed mice in a manner that was reduced with CSL311 treatment (FIG. 9B).

### Example 5: Airway Hyper-Responsiveness and Emphysema are Reduced by CSL311 Treatment

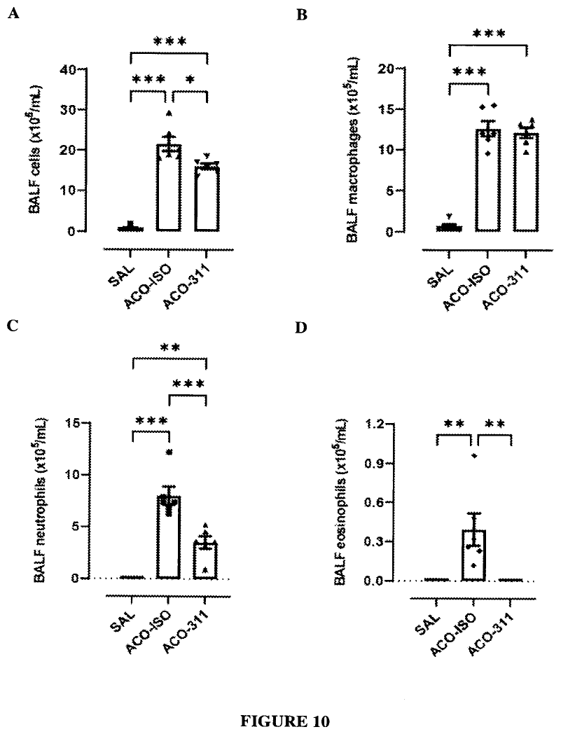

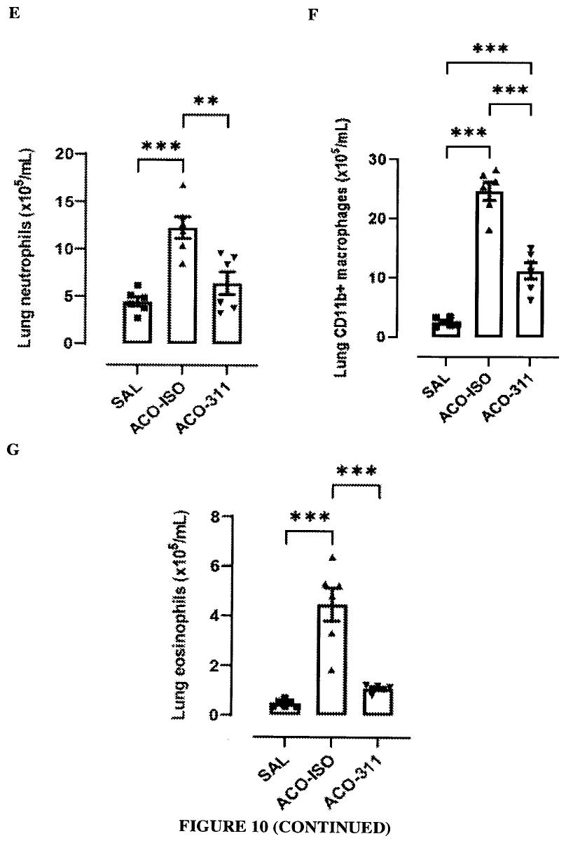

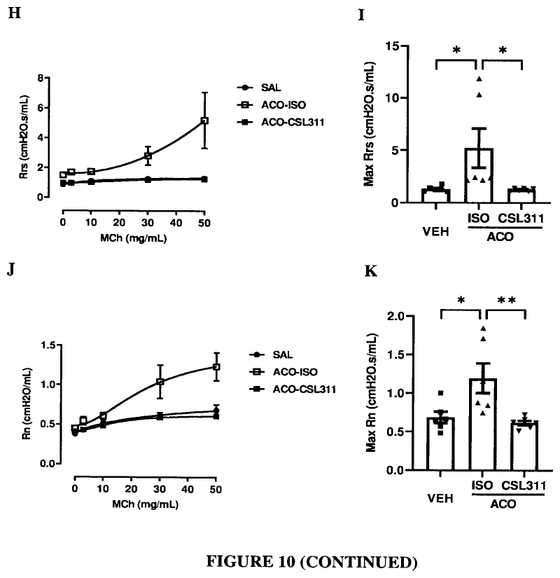

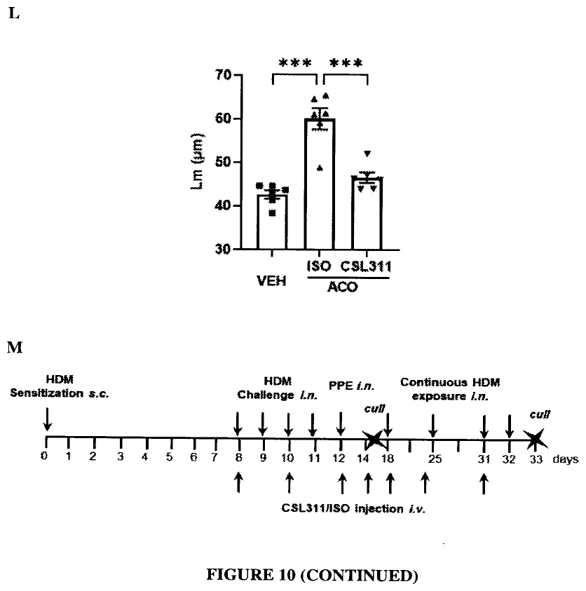

To investigate eosinophil-associated COPD/asthma overlap (ACO), female hβcTg mice were sensitised (s.c. injection) with house dust mite extract (HDM, Dermatophagoides pteronyssinus, Greer Laboratories, US; 100 μg emulsified in complete Freund's adjuvant, CFA) and further challenged with porcine pancreatic elastase (PPE, 0.5 U in saline, i.n.) and intranasal HDM (25 μg in saline) as shown in FIG. 10M. CSL311 or isotype control was administered to the HDM+PPE co-treated hβcTg mice (ACO) intravenously (i.v.) at 50 mg/kg on experimental day 8, day 10, day 12, day 14, day 18, day 25, and day 31, respectively. Mice were euthanized on experimental day 15 to assess the lung inflammation, or day 33 for lung function measurements and lung histology.

During the inflammatory phase (day 15), infiltration of immune cells to the BAL compartment (FIG. 10A), comprised macrophages (FIG. 10B), neutrophils (FIG. 10C) and eosinophils (FIG. 10D) was markedly increased in ACO mice. βc antagonism by CSL311 significantly reduced BALF neutrophils (FIG. 10C) and eosinophils (FIG. 10D). In the lung compartment, accumulation of neutrophils (FIG. 10E), CD11b+ macrophages (including IMs and EMs, FIG. 10F) and eosinophils (FIG. 10G) was increased. βc antagonism by CSL311 largely reduced the lung accumulation of all these immune cell types. On experimental day 33, airway hyper-responsiveness (AHR) measured by increased total respiratory resistance (Rrs, FIGS. 10H and I) and airway resistance (Rn, FIGS. 10J and K)) in ACO mice was also prevented by CSL311. At a histopathological level, airspace enlargement or emphysema measured by the increased mean linear intercept on H&E stain lung sections (Lm, FIG. 10L) was fully prevented by CSL311.

### Example 6: Human, Non-Interventional Analysis of CD131 as a Target for Reducing Inflammation in Chronic Airway Disease

Peripheral blood (PB) and sputum was collected from 50 asthmatics and 30 COPD patients. PB was also collected from 60 matched healthy volunteers (HV). RNA was extracted and analysed by RNA sequencing. Immune cell subsets were quantified, and baseline activation and receptor expression were assessed by flow cytometry or Wright's staining of sputum Cytospins. White blood cells (WBC) were isolated and stimulated with combined βc cytokines+/−CSL311, and activation was evaluated by flow cytometry. Serum protein levels were measured by Luminex assays.

CD131 was detected on key immune cell effectors and expression was maintained in disease. Several subsets were elevated in PB of patients with airway disease and these cells exhibited a more activated profile (Table 2). Airway infiltration of neutrophils and eosinophils was also demonstrated by sputum cell counts.

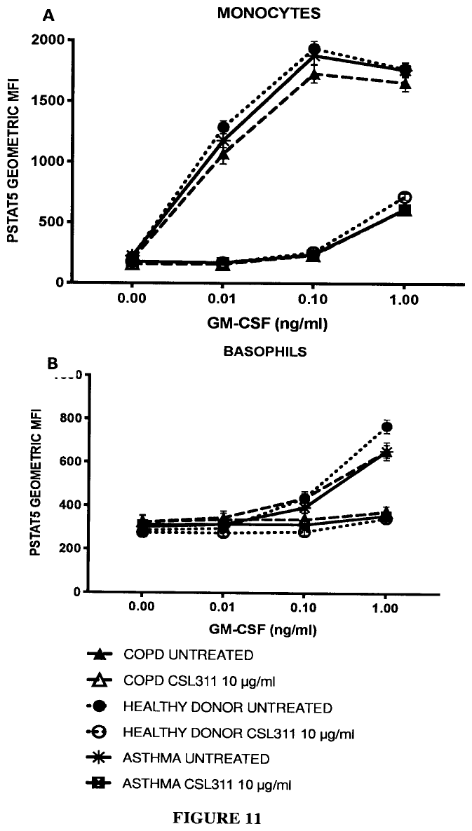

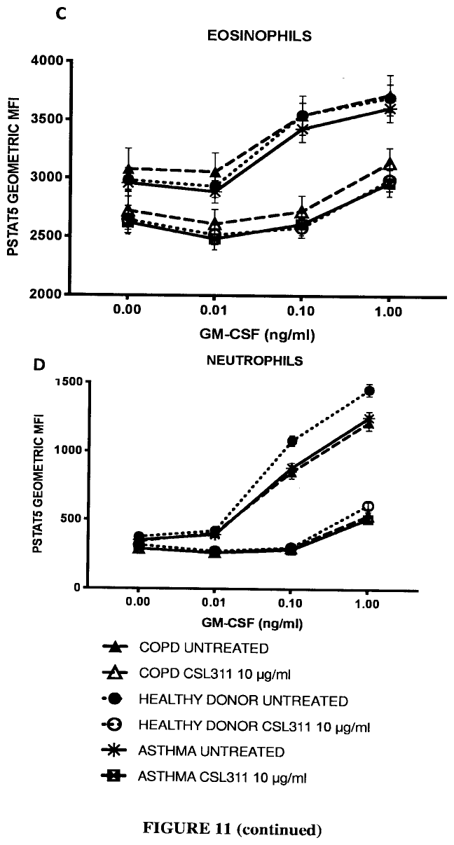

Induction of CD11b and CD35 expression and CD62L shedding was replicated ex-vivo by stimulating WBC with βc-cytokines IL-3, IL-5 and GM-CSF. As shown in Table 3, CSL311 pre-treatment was able to inhibit this response. In addition, GM-CSF-induced STAT5 activation was inhibited by CSL311 in a dose-dependent manner (FIG. 11).

Pathway analysis of the transcriptomic data derived from peripheral blood cells indicated over representation of transcripts related to inflammatory responses and immune cell trafficking in the peripheral blood from asthma and COPD patients.

Evidence of additional βc-cytokine activity was seen systemically, with serum CCL13 significantly elevated in asthmatics (p<0.0001) and CCL17 elevated in both asthma and COPD patients when compared to the matched healthy volunteers (p<0.001, p=0.011 respectively).

TABLE 2
CD131 expression and changes in peripheral blood cell frequency and activation in asthma and COPD

| | HV | ASTHMA | | | | | | COPD | | | | | |
|---|---|---|---|---|---|---|---|---|---|---|---|---|---|
| CELL TYPE | CD131+ | CD131+ | Count | CD11b | CD35 | CD62L | CD63 | CD131+ | Count | CD11b | CD35 | CD62L | CD63 |
| BASOPHIL | Yes | Yes | ↑ | ↑ | — | ↓ | ↓ | Yes | — | — | — | — | ↓ |
| | | — | p = | p = 0.0020 | | p = 0.0014 | p = | — | | | | | P = 0.0005 |
| | | | 0.0382 | | | | 0.0006 | | | | | | |
| EOSINOPHIL | Yes | Yes | ↑ | ↑ | — | ↓ | ↓ | Yes | — | ↑ | — | — | ↓ |
| | | ↑ | p = | p = 0.0001 | | p = 0.0011 | p = | — | | p <0.0001 | | | P = 0.0073 |
| | | p = | 0.0017 | | | | 0.0116 | | | | | | |
| | | 0.0091 | | | | | | | | | | | |
| MONOCYTE | Yes | Yes | — | ↑ | ↑ | — | ↓ | Yes | — | — | — | — | ↓ |
| | | — | | p = 0.0063 | (p = 0.0247) | | | — | | | | | P = 0.0004 |
| NEUTROPHIL | Yes | Yes | ↑ | ↑ | ↑ | ↓ | ↓ | Yes | ↑ | — | — | — | ↓ |
| | | — | p = | p <0.0001 | (p = 0.0332) | p = 0.0015 | | — | p = | | | | P = 0.0046 |
| | | | 0.0023 | | | | | | 0.001 | | | | |

Yes indicates CD131 expression above 200 ABC (antibodies bound per cell);
↑ indicates significant increase compared to healthy volunteers (HV);
↓ indicates significant decrease compared to HV;
— indicates no significant change.

TABLE 3
Ex vivo stimulation of white blood cells and effect of CSL311 pre-treatment compared to healthy volunteers

| | ASTHMA | | | | COPD | | | |
|---|---|---|---|---|---|---|---|---|
| | Basophils | Eosinophils | Neutrophils | Monocytes | Basophils | Eosinophils | Neutrophils | Monocytes |
| CD35 | Increased | Increased | Increased | — | Increased | Increased | Increased | — |
| Modulated by CSL311 | Yes | Yes | Yes | | Yes | Yes | Yes | |
| CD62L | — | Decreased | Decreased | Decreased | — | Decreased | Decreased | |
| Modulated by CSL311 | | Yes | Yes | | | Yes | Yes | |
| CD11b | Increased | Increased | Increased | — | Increased | Increased | Increased | — |
| Modulated by CSL311 | Yes | Yes | Yes | | Yes | Yes | Yes | |

WB markers analysed used a paired two-tailed t-test. The ability of IL-3/IL-5/GM-CSF to activate the cells was determined for the stimuli that induced >10% change in MFI and tested by uncorrected fisher's LSD test. The ability of CSL311 to modulate this activation was then tested for the stimuli that had >5% change in MFI and tested with a paired two-tailed t-test.
“—” no significant difference detected.

1. A method of depleting and/or reducing eosinophils in a subject suffering from chronic obstructive pulmonary disease (COPD), the method comprising administering to the subject a compound that binds to or specifically binds to CD131 and neutralises signaling by interleukin (IL) 3, IL-5 and granulocyte-macrophage colony stimulating factor (GM-CSF) in a subject.

2. A method of treating eosinophilia in a subject suffering from chronic obstructive pulmonary disease (COPD), the method comprising administering to the subject a compound that binds to or specifically binds to CD131 and neutralises signaling by interleukin (IL) 3, IL-5 and granulocyte-macrophage colony stimulating factor (GM-CSF) in a subject.

3. A method of treating eosinophil-associated chronic obstructive pulmonary disease (COPD), the method comprising administering to the subject a compound that binds to or specifically binds to CD131 and neutralises signaling by interleukin (IL) 3, IL-5 and granulocyte-macrophage colony stimulating factor (GM-CSF) in a subject.

4. The method of any one of claims 1 to 3, wherein the COPD is a smoking-related COPD.

5. The method of claim 4, wherein the smoking-related COPD is caused by chronic exposure to cigarette smoke.

6. The method of any one of claims 1 to 5, wherein the COPD is early COPD or pre-COPD.

7. The method of any one of claims 1 to 6, wherein administration of the compound reduces, or prevents an increase in, one or more or all of the following:
(i) total amount of white blood cells in the subject's blood;
(ii) amount of monocytes in the subject's blood;
(iii) amount of neutrophils in the subject's blood;
(iv) total amount of eosinophils in the subject's blood;
(v) total amount of cells in the bronchoalveolar (BAL) fluid of the subject's lung;
(vi) macrophage accumulation in the BAL fluid of the subject's lung;
(vii) neutrophil accumulation in BAL fluid of the subject's lung;
(viii) lymphocyte accumulation in the BAL fluid of the subject's lung;
(ix) neutrophil accumulation in the subject's lung;
(x) interstitial macrophage accumulation in the subject's lung;
(xi) eosinophil accumulation in the subject's lung;
(xii) CD11b+ exudative macrophage accumulation in the subject's lung;
(xiii) total amount of eosinophils in the subject's sputum; and/or
(xiv) inflammation of the subject's lung.

8. The method of any one of claims 1 to 7, wherein administration of the compound reduces, or prevents an increase in, one or more or all of the following:
(i) lung injury score in the subject;
(ii) albumin concentration in the BAL fluid of the subject's lung;
(iii) lactate dehydrogenase (LDH) levels in the BAL fluid of the subject's lung;
(iv) amount of dsDNA in the BAL fluid of the subject's lung; and/or
(v) gelatinase levels in the BAL fluid of the subject's lung.

9. The method of any one of claims 1 to 8, wherein administration of the compound reduces, or prevents an increase in, the level of expression of one or more genes in the subject's lung selected from the group consisting of IL-1α, IL-17a, IL-23a, C-X-C Motif Chemokine Ligand 1 (CXCL1), CXCL2, C-C Motif Chemokine Ligand 2 (CCL2), matrix metalloproteinase 12 (MMP-12), Found in Inflammatory Zone 1 (Fizz1) and GM-CSF.

10. The method of any one of claims 1 to 9, wherein the compound inhibits:
(a) GM-CSF-induced proliferation of TF-1 erythroleukemic cells with an IC₅₀ of at least 460 nM; and/or
(b) IL-5-induced proliferation of TF-1 erythroleukemic cells with an IC₅₀ of at least 1600 nM; and/or
(c) IL-3-induced proliferation of TF-1 erythroleukemic cells with an IC₅₀ of at least 10 nM.

11. The method of any one of claims 1 to 10, wherein the compound is a CD131-binding protein comprising an antigen binding site that:
(a) binds to or specifically binds to an epitope within site 2 of CD131 and neutralizes signaling by interleukin-3 (IL-3), interleukin-5 (IL-5) and granulocyte-macrophage colony stimulating factor (GM-CSF);
(b) binds to an epitope formed upon dimerization of two CD131 polypeptides; and
(c) binds to residues within domain 1 of a CD131 polypeptide and residues within domain 4 of another CD131 polypeptide.

12. The method of claim 11, wherein the epitope comprises one or more (or all) of amino acids corresponding to residues 39 and 103 of one CD131 polypeptide and residues 338, 365, 367 and 368 of the other CD131 polypeptide.

13. The method of any one of claims 1 to 12, wherein the compound is a CD131-binding protein that binds to one or more (or all) of the following mutant polypeptide(s):
(i) a mutant polypeptide comprising a sequence set forth in SEQ ID NO: 119;
(ii) a mutant polypeptide comprising a sequence set forth in SEQ ID NO: 123;
(iii) a mutant polypeptide comprising a sequence set forth in SEQ ID NO: 124;
(iv) a mutant polypeptide comprising a sequence set forth in SEQ ID NO: 135;
(v) a mutant polypeptide comprising a sequence set forth in SEQ ID NO: 131;
(vi) a mutant polypeptide comprising a sequence set forth in SEQ ID NO: 136;
(vii) a mutant polypeptide comprising a sequence set forth in SEQ ID NO: 137;
(viii) a mutant polypeptide comprising a sequence set forth in SEQ ID NO: 139;
(ix) a mutant polypeptide comprising a sequence set forth in SEQ ID NO: 145, at a level that is reduced compared to the level of binding of the CD131-binding protein to a polypeptide comprising a sequence set forth in SEQ ID NO: 192.

14. The method of any one of claims 1 to 13, wherein the compound is a CD131-binding protein comprising a Fv.

15. The method of claim 14, wherein the CD131-binding protein comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

16. The method of any one of claims 1 to 15, wherein the compound is a CD131-binding protein comprising an antigen binding domain of an antibody, wherein the antigen binding domain binds to or specifically binds to CD131, and wherein the CD131-binding protein competitively inhibits binding of antibody 9A2 (comprising a light chain variable region (V_{L}) comprising a sequence set forth in SEQ ID NO: 5 and a heavy chain variable region (V_{H}) comprising a sequence set forth in SEQ ID NO: 20).

17. The method of any one of claims 1 to 16, wherein the compound is a CD131-binding protein comprising an antigen binding domain of an antibody, wherein the antigen binding domain binds to a polypeptide comprising a sequence set forth in SEQ ID NO: 127 with a higher affinity than it does to a polypeptide comprising a sequence set forth in SEQ ID NO: 192.

18. The method of any one of claims 1 to 17, wherein the compound is a CD131-binding protein comprising an antigen binding domain of an antibody, wherein the antigen binding domain comprises a V_{H} and a V_{L}, wherein:
(A) (i) the V_{H} comprises a CDR1 comprising a sequence set forth between amino acids 26-35 of SEQ ID NO: 180, a CDR2 comprising a sequence set forth between amino acids 50-66 of SEQ ID NO: 180 and a CDR3 comprising a sequence set forth between amino acids 99-106 of SEQ ID NO: 180 or the V_{H} comprising a sequence set forth in SEQ ID NO: 180; and
(ii) the V_{L} comprising a CDR1 comprising a sequence set forth between amino acids 24-34 of SEQ ID NO: 177, a CDR2 comprising a sequence set forth between amino acids 44-51 of SEQ ID NO: 177 and a CDR3 comprising a sequence set forth between amino acids 89-97 of SEQ ID NO: 177 or the V_{L} comprising a sequence set forth in SEQ ID NO: 177; or
(B) the V_{H} comprises a sequence set forth in SEQ ID NO: 193 and the V_{L} comprises a sequence set forth in SEQ ID NO: 5.

19. The method of any one of claims 1 to 18, wherein the compound is a CD131-binding protein comprising an antigen binding domain of an antibody, wherein the antigen binding domain comprises:
(i) a V_{H} comprising CDRs 1, 2 and 3 of a sequence set forth in SEQ ID NO: 20 and a V_{L} comprising CDRs 1, 2 and 3 of a sequence set forth in SEQ ID NO: 5;
(ii) a V_{H} comprising a sequence set forth in SEQ ID NO: 20 and a V_{L} comprising a sequence set forth in SEQ ID NO: 5;
(iii) a V_{H} comprising CDRs 1, 2 and 3 of a sequence set forth in SEQ ID NO: 20 and a V_{L} comprising CDRs 1, 2 and 3 of a sequence set forth in SEQ ID NO: 6;
(iv) a V_{H} comprising a sequence set forth in SEQ ID NO: 20 and a V_{L} comprising a sequence set forth in SEQ ID NO: 6;
(v) a V_{H} comprising CDRs 1, 2 and 3 of a sequence set forth in SEQ ID NO: 20 and a V_{L} comprising CDRs 1, 2 and 3 of a sequence set forth in SEQ ID NO: 7;
(vi) a V_{H} comprising a sequence set forth in SEQ ID NO: 20 and a V_{L} comprising a sequence set forth in SEQ ID NO: 7;
(vii) a V_{H} comprising CDRs 1, 2 and 3 of a sequence set forth in SEQ ID NO: 20 and a V_{L} comprising CDRs 1, 2 and 3 of a sequence set forth in SEQ ID NO: 8;
(viii) a V_{H} comprising a sequence set forth in SEQ ID NO: 20 and a V_{L} comprising a sequence set forth in SEQ ID NO: 8;
(ix) a V_{H} comprising CDRs 1, 2 and 3 of a sequence set forth in SEQ ID NO: 20 and a V_{L} comprising CDRs 1, 2 and 3 of a sequence set forth in SEQ ID NO: 9;
(x) a V_{H} comprising a sequence set forth in SEQ ID NO: 20 and a V_{L} comprising a sequence set forth in SEQ ID NO: 9;
(xi) a V_{H} comprising CDRs 1, 2 and 3 of a sequence set forth in SEQ ID NO: 20 and a V_{L} comprising CDRs 1, 2 and 3 of a sequence set forth in SEQ ID NO: 10;
(xii) a V_{H} comprising a sequence set forth in SEQ ID NO: 20 and a V_{L} comprising a sequence set forth in SEQ ID NO: 10;
(xiii) a V_{H} comprising CDRs 1, 2 and 3 of a sequence set forth in SEQ ID NO: 20 and a V_{L} comprising CDRs 1, 2 and 3 of a sequence set forth in SEQ ID NO: 11;
(xiv) a V_{H} comprising a sequence set forth in SEQ ID NO: 20 and a V_{L} comprising a sequence set forth in SEQ ID NO: 11;
(xv) a V_{H} comprising CDRs 1, 2 and 3 of a sequence set forth in SEQ ID NO: 20 and a V_{L} comprising CDRs 1, 2 and 3 of a sequence set forth in SEQ ID NO: 12;
(xvi) a V_{H} comprising a sequence set forth in SEQ ID NO: 20 and a V_{L} comprising a sequence set forth in SEQ ID NO: 12;
(xvii) a V_{H} comprising CDRs 1, 2 and 3 of a sequence set forth in SEQ ID NO: 20 and a V_{L} comprising CDRs 1, 2 and 3 of a sequence set forth in SEQ ID NO: 13;
(xviii) a V_{H} comprising a sequence set forth in SEQ ID NO: 20 and a V_{L} comprising a sequence set forth in SEQ ID NO: 13;
(xix) a V_{H} comprising CDRs 1, 2 and 3 of a sequence set forth in SEQ ID NO: 20 and a V_{L} comprising CDRs 1, 2 and 3 of a sequence set forth in SEQ ID NO: 14;
(xx) a V_{H} comprising a sequence set forth in SEQ ID NO: 20 and a V_{L} comprising a sequence set forth in SEQ ID NO: 14;
(xxi) a V_{H} comprising CDRs 1, 2 and 3 of a sequence set forth in SEQ ID NO: 20 and a V_{L} comprising CDRs 1, 2 and 3 of a sequence set forth in SEQ ID NO: 15;
(xxii) a V_{H} comprising a sequence set forth in SEQ ID NO: 20 and a V_{L} comprising a sequence set forth in SEQ ID NO: 15;
(xxiii) a V_{H} comprising CDRs 1, 2 and 3 of a sequence set forth in SEQ ID NO: 21 and a V_{L} comprising CDRs 1, 2 and 3 of a sequence set forth in SEQ ID NO: 5;
(xxiv) a V_{H} comprising a sequence set forth in SEQ ID NO: 21 and a V_{L} comprising a sequence set forth in SEQ ID NO: 5;
(xxv) a V_{H} comprising CDRs 1, 2 and 3 of a sequence set forth in SEQ ID NO: 22 and a V_{L} comprising CDRs 1, 2 and 3 of a sequence set forth in SEQ ID NO: 5;
(xxvi) a V_{H} comprising a sequence set forth in SEQ ID NO: 22 and a V_{L} comprising a sequence set forth in SEQ ID NO: 5;
(xxvii) a V_{H} comprising CDRs 1, 2 and 3 of a sequence set forth in SEQ ID NO: 23 and a V_{L} comprising CDRs 1, 2 and 3 of a sequence set forth in SEQ ID NO: 5;
(xxviii) a V_{H} comprising a sequence set forth in SEQ ID NO: 23 and a V_{L} comprising a sequence set forth in SEQ ID NO: 5;
(xxix) a V_{H} comprising CDRs 1, 2 and 3 of a sequence set forth in SEQ ID NO: 24 and a V_{L} comprising CDRs 1, 2 and 3 of a sequence set forth in SEQ ID NO: 5;
(xxx) a V_{H} comprising a sequence set forth in SEQ ID NO: 24 and a V_{L} comprising a sequence set forth in SEQ ID NO: 5;
(xxxi) a V_{H} comprising CDRs 1, 2 and 3 of a sequence set forth in SEQ ID NO: 25 and a V_{L} comprising CDRs 1, 2 and 3 of a sequence set forth in SEQ ID NO: 5;
(xxxii) a V_{H} comprising a sequence set forth in SEQ ID NO: 25 and a V_{L} comprising a sequence set forth in SEQ ID NO: 5;
(xxxiii) a V_{H} comprising CDRs 1, 2 and 3 of a sequence set forth in SEQ ID NO: 26 and a V_{L} comprising CDRs 1, 2 and 3 of a sequence set forth in SEQ ID NO: 5;
(xxxiv) a V_{H} comprising a sequence set forth in SEQ ID NO: 26 and a V_{L} comprising a sequence set forth in SEQ ID NO: 5;
(xxxv) a V_{H} comprising CDRs 1, 2 and 3 of a sequence set forth in SEQ ID NO: 27 and a V_{L} comprising CDRs 1, 2 and 3 of a sequence set forth in SEQ ID NO: 5;
(xxxvi) a V_{H} comprising a sequence set forth in SEQ ID NO: 27 and a V_{L} comprising a sequence set forth in SEQ ID NO: 5;
(xxxvii) a V_{H} comprising CDRs 1, 2 and 3 of a sequence set forth in SEQ ID NO: 28 and a V_{L} comprising CDRs 1, 2 and 3 of a sequence set forth in SEQ ID NO: 5;
(xxxviii) a V_{H} comprising a sequence set forth in SEQ ID NO: 28 and a V_{L} comprising a sequence set forth in SEQ ID NO: 5;
(xxxix) a V_{H} comprising CDRs 1, 2 and 3 of a sequence set forth in SEQ ID NO: 29 and a V_{L} comprising CDRs 1, 2 and 3 of a sequence set forth in SEQ ID NO: 5;
(xl) a V_{H} comprising a sequence set forth in SEQ ID NO: 29 and a V_{L} comprising a sequence set forth in SEQ ID NO: 5;
(xli) a V_{H} comprising CDRs 1, 2 and 3 of a sequence set forth in SEQ ID NO: 30 and a V_{L} comprising CDRs 1, 2 and 3 of a sequence set forth in SEQ ID NO: 5;
(xlii) a V_{H} comprising a sequence set forth in SEQ ID NO: 30 and a V_{L} comprising a sequence set forth in SEQ ID NO: 5;
(xliii) a V_{H} comprising CDRs 1, 2 and 3 of a sequence set forth in SEQ ID NO: 31 and a V_{L} comprising CDRs 1, 2 and 3 of a sequence set forth in SEQ ID NO: 5;
(xliv) a V_{H} comprising a sequence set forth in SEQ ID NO: 31 and a V_{L} comprising a sequence set forth in SEQ ID NO: 5;
(xlv) a V_{H} comprising CDRs 1, 2 and 3 of a sequence set forth in SEQ ID NO: 32 and a V_{L} comprising CDRs 1, 2 and 3 of a sequence set forth in SEQ ID NO: 5;
(xlvi) a V_{H} comprising a sequence set forth in SEQ ID NO: 32 and a V_{L} comprising a sequence set forth in SEQ ID NO: 5;
(xlvii) a V_{H} comprising CDRs 1, 2 and 3 of a sequence set forth in SEQ ID NO: 33 and a V_{L} comprising CDRs 1, 2 and 3 of a sequence set forth in SEQ ID NO: 5;
(xlviii) a V_{H} comprising a sequence set forth in SEQ ID NO: 33 and a V_{L} comprising a sequence set forth in SEQ ID NO: 5;
(xlix) a V_{H} comprising CDRs 1, 2 and 3 of a sequence set forth in SEQ ID NO: 34 and a V_{L} comprising CDRs 1, 2 and 3 of a sequence set forth in SEQ ID NO: 5;
(1) a V_{H} comprising a sequence set forth in SEQ ID NO: 34 and a V_{L} comprising a sequence set forth in SEQ ID NO: 5;
(li) a V_{H} comprising CDRs 1, 2 and 3 of a sequence set forth in SEQ ID NO: 35 and a V_{L} comprising CDRs 1, 2 and 3 of a sequence set forth in SEQ ID NO: 5;
(lii) a V_{H} comprising a sequence set forth in SEQ ID NO: 35 and a V_{L} comprising a sequence set forth in SEQ ID NO: 5;
(liii) a V_{H} comprising CDRs 1, 2 and 3 of a sequence set forth in SEQ ID NO: 36 and a V_{L} comprising CDRs 1, 2 and 3 of a sequence set forth in SEQ ID NO: 5;
(liv) a V_{H} comprising a sequence set forth in SEQ ID NO: 36 and a V_{L} comprising a sequence set forth in SEQ ID NO: 5;
(lv) a V_{H} comprising CDRs 1, 2 and 3 of a sequence set forth in SEQ ID NO: 37 and a V_{L} comprising CDRs 1, 2 and 3 of a sequence set forth in SEQ ID NO: 5;
(lvi) a V_{H} comprising a sequence set forth in SEQ ID NO: 37 and a V_{L} comprising a sequence set forth in SEQ ID NO: 5;
(lvii) a V_{H} comprising CDRs 1, 2 and 3 of a sequence set forth in SEQ ID NO: 38 and a V_{L} comprising CDRs 1, 2 and 3 of a sequence set forth in SEQ ID NO: 5;
(lviii) a V_{H} comprising a sequence set forth in SEQ ID NO: 38 and a V_{L} comprising a sequence set forth in SEQ ID NO: 5;
(lix) a V_{H} comprising CDRs 1, 2 and 3 of a sequence set forth in SEQ ID NO: 39 and a V_{L} comprising CDRs 1, 2 and 3 of a sequence set forth in SEQ ID NO: 5;
(lx) a V_{H} comprising a sequence set forth in SEQ ID NO: 39 and a V_{L} comprising a sequence set forth in SEQ ID NO: 5;
(lxi) a V_{H} comprising CDRs 1, 2 and 3 of a sequence set forth in SEQ ID NO: 40 and a V_{L} comprising CDRs 1, 2 and 3 of a sequence set forth in SEQ ID NO: 5;
(lxii) a V_{H} comprising a sequence set forth in SEQ ID NO: 40 and a V_{L} comprising a sequence set forth in SEQ ID NO: 5;
(lxiii) a V_{H} comprising CDRs 1, 2 and 3 of a sequence set forth in SEQ ID NO: 41 and a V_{L} comprising CDRs 1, 2 and 3 of a sequence set forth in SEQ ID NO: 5;
(lxiv) a V_{H} comprising a sequence set forth in SEQ ID NO: 41 and a V_{L} comprising a sequence set forth in SEQ ID NO: 5;
(lxv) a V_{H} comprising CDRs 1, 2 and 3 of a sequence set forth in SEQ ID NO: 42 and a V_{L} comprising CDRs 1, 2 and 3 of a sequence set forth in SEQ ID NO: 5;
(lxvi) a V_{H} comprising a sequence set forth in SEQ ID NO: 42 and a V_{L} comprising a sequence set forth in SEQ ID NO: 5;
(lxvii) a V_{H} comprising CDRs 1, 2 and 3 of a sequence set forth in SEQ ID NO: 43 and a V_{L} comprising CDRs 1, 2 and 3 of a sequence set forth in SEQ ID NO: 5;
(lxviii) a V_{H} comprising a sequence set forth in SEQ ID NO: 43 and a V_{L} comprising a sequence set forth in SEQ ID NO: 5;
(lxix) a V_{H} comprising CDRs 1, 2 and 3 of a sequence set forth in SEQ ID NO: 44 and a V_{L} comprising CDRs 1, 2 and 3 of a sequence set forth in SEQ ID NO: 5;
(lxx) a V_{H} comprising a sequence set forth in SEQ ID NO: 44 and a V_{L} comprising a sequence set forth in SEQ ID NO: 5;
(lxxi) a V_{H} comprising CDRs 1, 2 and 3 of a sequence set forth in SEQ ID NO: 45 and a V_{L} comprising CDRs 1, 2 and 3 of a sequence set forth in SEQ ID NO: 5;
(lxxii) a V_{H} comprising a sequence set forth in SEQ ID NO: 45 and a V_{L} comprising a sequence set forth in SEQ ID NO: 5;
(lxxiii) a V_{H} comprising CDRs 1, 2 and 3 of a sequence set forth in SEQ ID NO: 46 and a V_{L} comprising CDRs 1, 2 and 3 of a sequence set forth in SEQ ID NO: 5;
(lxxiv) a V_{H} comprising a sequence set forth in SEQ ID NO: 46 and a V_{L} comprising a sequence set forth in SEQ ID NO: 5;
(lxxv) a V_{H} comprising CDRs 1, 2 and 3 of a sequence set forth in SEQ ID NO: 47 and a V_{L} comprising CDRs 1, 2 and 3 of a sequence set forth in SEQ ID NO: 5;
(lxxvi) a V_{H} comprising a sequence set forth in SEQ ID NO: 47 and a V_{L} comprising a sequence set forth in SEQ ID NO: 5;
(lxxvii) a V_{H} comprising CDRs 1, 2 and 3 of a sequence set forth in SEQ ID NO: 48 and a V_{L} comprising CDRs 1, 2 and 3 of a sequence set forth in SEQ ID NO: 5;
(lxxviii) a V_{H} comprising a sequence set forth in SEQ ID NO: 48 and a V_{L} comprising a sequence set forth in SEQ ID NO: 5;
(lxxix) a V_{H} comprising CDRs 1, 2 and 3 of a sequence set forth in SEQ ID NO: 49 and a V_{L} comprising CDRs 1, 2 and 3 of a sequence set forth in SEQ ID NO: 5;
(lxxx) a V_{H} comprising a sequence set forth in SEQ ID NO: 49 and a V_{L} comprising a sequence set forth in SEQ ID NO: 5;
(lxxxi) a V_{H} comprising CDRs 1, 2 and 3 of a sequence set forth in SEQ ID NO: 50 and a V_{L} comprising CDRs 1, 2 and 3 of a sequence set forth in SEQ ID NO: 5;
(lxxxii) a V_{H} comprising a sequence set forth in SEQ ID NO: 50 and a V_{L} comprising a sequence set forth in SEQ ID NO: 5;
(lxxxiii) a V_{H} comprising CDRs 1, 2 and 3 of a sequence set forth in SEQ ID NO: 51 and a V_{L} comprising CDRs 1, 2 and 3 of a sequence set forth in SEQ ID NO: 5;
(lxxxiv) a V_{H} comprising a sequence set forth in SEQ ID NO: 51 and a V_{L} comprising a sequence set forth in SEQ ID NO: 5;
(lxxxv) a V_{H} comprising CDRs 1, 2 and 3 of a sequence set forth in SEQ ID NO: 52 and a V_{L} comprising CDRs 1, 2 and 3 of a sequence set forth in SEQ ID NO: 5;
(lxxxvi) a V_{H} comprising a sequence set forth in SEQ ID NO: 52 and a V_{L} comprising a sequence set forth in SEQ ID NO: 5;
(lxxxvii) a V_{H} comprising CDRs 1, 2 and 3 of a sequence set forth in SEQ ID NO: 53 and a V_{L} comprising CDRs 1, 2 and 3 of a sequence set forth in SEQ ID NO: 5;
(lxxxviii) a V_{H} comprising a sequence set forth in SEQ ID NO: 53 and a V_{L} comprising a sequence set forth in SEQ ID NO: 5;
(lxxxix) a V_{H} comprising CDRs 1, 2 and 3 of a sequence set forth in SEQ ID NO: 54 and a V_{L} comprising CDRs 1, 2 and 3 of a sequence set forth in SEQ ID NO: 5;
(xc) a V_{H} comprising a sequence set forth in SEQ ID NO: 54 and a V_{L} comprising a sequence set forth in SEQ ID NO: 5;
(xci) a V_{H} comprising CDRs 1, 2 and 3 of a sequence set forth in SEQ ID NO: 55 and a V_{L} comprising CDRs 1, 2 and 3 of a sequence set forth in SEQ ID NO: 5;
(xcii) a V_{H} comprising a sequence set forth in SEQ ID NO: 55 and a V_{L} comprising a sequence set forth in SEQ ID NO: 5;
(xciii) a V_{H} comprising CDRs 1, 2 and 3 of a sequence set forth in SEQ ID NO: 56 and a V_{L} comprising CDRs 1, 2 and 3 of a sequence set forth in SEQ ID NO: 5;
(xciv) a V_{H} comprising a sequence set forth in SEQ ID NO: 56 and a V_{L} comprising a sequence set forth in SEQ ID NO: 5;
(xcv) a V_{H} comprising CDRs 1, 2 and 3 of a sequence set forth in SEQ ID NO: 57 and a V_{L} comprising CDRs 1, 2 and 3 of a sequence set forth in SEQ ID NO: 5;
(xcvi) a V_{H} comprising a sequence set forth in SEQ ID NO: 57 and a V_{L} comprising a sequence set forth in SEQ ID NO: 5;
(xcvii) a V_{H} comprising CDRs 1, 2 and 3 of a sequence set forth in SEQ ID NO: 58 and a V_{L} comprising CDRs 1, 2 and 3 of a sequence set forth in SEQ ID NO: 5;
(xcviii) a V_{H} comprising a sequence set forth in SEQ ID NO: 58 and a V_{L} comprising a sequence set forth in SEQ ID NO: 5;
(xcix) a V_{H} comprising CDRs 1, 2 and 3 of a sequence set forth in SEQ ID NO: 59 and a V_{L} comprising CDRs 1, 2 and 3 of a sequence set forth in SEQ ID NO: 5;
(c) a V_{H} comprising a sequence set forth in SEQ ID NO: 59 and a V_{L} comprising a sequence set forth in SEQ ID NO: 5;
(ci) a V_{H} comprising CDRs 1, 2 and 3 of a sequence set forth in SEQ ID NO: 60 and a V_{L} comprising CDRs 1, 2 and 3 of a sequence set forth in SEQ ID NO: 5;
(cii) a V_{H} comprising a sequence set forth in SEQ ID NO: 60 and a V_{L} comprising a sequence set forth in SEQ ID NO: 5;
(ciii) a V_{H} comprising CDRs 1, 2 and 3 of a sequence set forth in SEQ ID NO: 61 and a V_{L} comprising CDRs 1, 2 and 3 of a sequence set forth in SEQ ID NO: 5;
(civ) a V_{H} comprising a sequence set forth in SEQ ID NO: 61 and a V_{L} comprising a sequence set forth in SEQ ID NO: 5;
(cv) a V_{H} comprising CDRs 1, 2 and 3 of a sequence set forth in SEQ ID NO: 62 and a V_{L} comprising CDRs 1, 2 and 3 of a sequence set forth in SEQ ID NO: 5;
(cvi) a V_{H} comprising a sequence set forth in SEQ ID NO: 62 and a V_{L} comprising a sequence set forth in SEQ ID NO: 5;
(cvii) a V_{H} comprising CDRs 1, 2 and 3 of a sequence set forth in SEQ ID NO: 63 and a V_{L} comprising CDRs 1, 2 and 3 of a sequence set forth in SEQ ID NO: 5;
(cviii) a V_{H} comprising a sequence set forth in SEQ ID NO: 63 and a V_{L} comprising a sequence set forth in SEQ ID NO: 5;
(cix) a V_{H} comprising CDRs 1, 2 and 3 of a sequence set forth in SEQ ID NO: 64 and a V_{L} comprising CDRs 1, 2 and 3 of a sequence set forth in SEQ ID NO: 5;
(cx) a V_{H} comprising a sequence set forth in SEQ ID NO: 64 and a V_{L} comprising a sequence set forth in SEQ ID NO: 5;
(cxi) a V_{H} comprising CDRs 1, 2 and 3 of a sequence set forth in SEQ ID NO: 65 and a V_{L} comprising CDRs 1, 2 and 3 of a sequence set forth in SEQ ID NO: 5;
(cxii) a V_{H} comprising a sequence set forth in SEQ ID NO: 65 and a V_{L} comprising a sequence set forth in SEQ ID NO: 5;
(cxiii) a V_{H} comprising CDRs 1, 2 and 3 of a sequence set forth in SEQ ID NO: 66 and a V_{L} comprising CDRs 1, 2 and 3 of a sequence set forth in SEQ ID NO: 5;
(cxiv) a V_{H} comprising a sequence set forth in SEQ ID NO: 66 and a V_{L} comprising a sequence set forth in SEQ ID NO: 5;
(cxv) a V_{H} comprising CDRs 1, 2 and 3 of a sequence set forth in SEQ ID NO: 67 and a V_{L} comprising CDRs 1, 2 and 3 of a sequence set forth in SEQ ID NO: 5;
(cxvi) a V_{H} comprising a sequence set forth in SEQ ID NO: 67 and a V_{L} comprising a sequence set forth in SEQ ID NO: 5;
(cxvii) a V_{H} comprising CDRs 1, 2 and 3 of a sequence set forth in SEQ ID NO: 68 and a V_{L} comprising CDRs 1, 2 and 3 of a sequence set forth in SEQ ID NO: 5;
(cxviii) a V_{H} comprising a sequence set forth in SEQ ID NO: 68 and a V_{L} comprising a sequence set forth in SEQ ID NO: 5;
(cxix) a V_{H} comprising CDRs 1, 2 and 3 of a sequence set forth in SEQ ID NO: 69 and a V_{L} comprising CDRs 1, 2 and 3 of a sequence set forth in SEQ ID NO: 5;
(cxx) a V_{H} comprising a sequence set forth in SEQ ID NO: 69 and a V_{L} comprising a sequence set forth in SEQ ID NO: 5;
(cxxi) a V_{H} comprising CDRs 1, 2 and 3 of a sequence set forth in SEQ ID NO: 70 and a V_{L} comprising CDRs 1, 2 and 3 of a sequence set forth in SEQ ID NO: 5;
(cxxii) a V_{H} comprising a sequence set forth in SEQ ID NO: 70 and a V_{L} comprising a sequence set forth in SEQ ID NO: 5;
(cxxiii) a V_{H} comprising CDRs 1, 2 and 3 of a sequence set forth in SEQ ID NO: 71 and a V_{L} comprising CDRs 1, 2 and 3 of a sequence set forth in SEQ ID NO: 5;
(cxxiv) a V_{H} comprising a sequence set forth in SEQ ID NO: 71 and a V_{L} comprising a sequence set forth in SEQ ID NO: 5;
(cxxv) a V_{H} comprising CDRs 1, 2 and 3 of a sequence set forth in SEQ ID NO: 72 and a V_{L} comprising CDRs 1, 2 and 3 of a sequence set forth in SEQ ID NO: 5;
(cxxvi) a V_{H} comprising a sequence set forth in SEQ ID NO: 72 and a V_{L} comprising a sequence set forth in SEQ ID NO: 5;
(cxxvii) a V_{H} comprising CDRs 1, 2 and 3 of a sequence set forth in SEQ ID NO: 73 and a V_{L} comprising CDRs 1, 2 and 3 of a sequence set forth in SEQ ID NO: 5;
(cxxviii) a V_{H} comprising a sequence set forth in SEQ ID NO: 73 and a V_{L} comprising a sequence set forth in SEQ ID NO: 5;
(cxxix) a V_{H} comprising CDRs 1, 2 and 3 of a sequence set forth in SEQ ID NO: 75 and a V_{L} comprising CDRs 1, 2 and 3 of a sequence set forth in SEQ ID NO: 5;
(cxxx) a V_{H} comprising a sequence set forth in SEQ ID NO: 75 and a V_{L} comprising a sequence set forth in SEQ ID NO: 5;
(cxxxi) a V_{H} comprising CDRs 1, 2 and 3 of a sequence set forth in SEQ ID NO: 76 and a V_{L} comprising CDRs 1, 2 and 3 of a sequence set forth in SEQ ID NO: 5;
(cxxxii) a V_{H} comprising a sequence set forth in SEQ ID NO: 76 and a V_{L} comprising a sequence set forth in SEQ ID NO: 5;
(cxxxiii) a V_{H} comprising CDRs 1, 2 and 3 of a sequence set forth in SEQ ID NO: 77 and a V_{L} comprising CDRs 1, 2 and 3 of a sequence set forth in SEQ ID NO: 5;
(cxxxiv) a V_{H} comprising a sequence set forth in SEQ ID NO: 77 and a V_{L} comprising a sequence set forth in SEQ ID NO: 5;
(cxxxv) a V_{H} comprising CDRs 1, 2 and 3 of a sequence set forth in SEQ ID NO: 78 and a V_{L} comprising CDRs 1, 2 and 3 of a sequence set forth in SEQ ID NO: 5;
(cxxxvi) a V_{H} comprising a sequence set forth in SEQ ID NO: 78 and a V_{L} comprising a sequence set forth in SEQ ID NO: 5;
(cxxxvii) a V_{H} comprising CDRs 1, 2 and 3 of a sequence set forth in SEQ ID NO: 79 and a V_{L} comprising CDRs 1, 2 and 3 of a sequence set forth in SEQ ID NO: 5;
(cxxxviii) a V_{H} comprising a sequence set forth in SEQ ID NO: 79 and a V_{L} comprising a sequence set forth in SEQ ID NO: 5;
(cxxxix) a V_{H} comprising CDRs 1, 2 and 3 of a sequence set forth in SEQ ID NO: 80 and a V_{L} comprising CDRs 1, 2 and 3 of a sequence set forth in SEQ ID NO: 5;
(cxl) a V_{H} comprising a sequence set forth in SEQ ID NO: 80 and a V_{L} comprising a sequence set forth in SEQ ID NO: 5;
(cxli) a V_{H} comprising CDRs 1, 2 and 3 of a sequence set forth in SEQ ID NO: 81 and a V_{L} comprising CDRs 1, 2 and 3 of a sequence set forth in SEQ ID NO: 5;
(cxlii) a V_{H} comprising a sequence set forth in SEQ ID NO: 81 and a V_{L} comprising a sequence set forth in SEQ ID NO: 5;
(cxliii) a V_{H} comprising CDRs 1, 2 and 3 of a sequence set forth in SEQ ID NO: 82 and a V_{L} comprising CDRs 1, 2 and 3 of a sequence set forth in SEQ ID NO: 5;
(cxliv) a V_{H} comprising a sequence set forth in SEQ ID NO: 82 and a V_{L} comprising a sequence set forth in SEQ ID NO: 5;
(cxlv) a V_{H} comprising CDRs 1, 2 and 3 of a sequence set forth in SEQ ID NO: 83 and a V_{L} comprising CDRs 1, 2 and 3 of a sequence set forth in SEQ ID NO: 5;
(cxlvi) a V_{H} comprising a sequence set forth in SEQ ID NO: 83 and a V_{L} comprising a sequence set forth in SEQ ID NO: 5;
(cxlvii) a V_{H} comprising CDRs 1, 2 and 3 of a sequence set forth in SEQ ID NO: 84 and a V_{L} comprising CDRs 1, 2 and 3 of a sequence set forth in SEQ ID NO: 5;
(cxlviii) a V_{H} comprising a sequence set forth in SEQ ID NO: 84 and a V_{L} comprising a sequence set forth in SEQ ID NO: 5;
(cxlix) a V_{H} comprising CDRs 1, 2 and 3 of a sequence set forth in SEQ ID NO: 85 and a V_{L} comprising CDRs 1, 2 and 3 of a sequence set forth in SEQ ID NO: 5;
(cl) a V_{H} comprising a sequence set forth in SEQ ID NO: 85 and a V_{L} comprising a sequence set forth in SEQ ID NO: 5;
(cli) a V_{H} comprising CDRs 1, 2 and 3 of a sequence set forth in SEQ ID NO: 86 and a V_{L} comprising CDRs 1, 2 and 3 of a sequence set forth in SEQ ID NO: 5;
(clii) a V_{H} comprising a sequence set forth in SEQ ID NO: 86 and a V_{L} comprising a sequence set forth in SEQ ID NO: 5;
(cliii) a V_{H} comprising CDRs 1, 2 and 3 of a sequence set forth in SEQ ID NO: 87 and a V_{L} comprising CDRs 1, 2 and 3 of a sequence set forth in SEQ ID NO: 5;
(cliv) a V_{H} comprising a sequence set forth in SEQ ID NO: 87 and a V_{L} comprising a sequence set forth in SEQ ID NO: 5;
(clv) a V_{H} comprising CDRs 1, 2 and 3 of a sequence set forth in SEQ ID NO: 88 and a V_{L} comprising CDRs 1, 2 and 3 of a sequence set forth in SEQ ID NO: 5;
(clvi) a V_{H} comprising a sequence set forth in SEQ ID NO: 88 and a V_{L} comprising a sequence set forth in SEQ ID NO: 5;
(clvii) a V_{H} comprising CDRs 1, 2 and 3 of a sequence set forth in SEQ ID NO: 89 and a V_{L} comprising CDRs 1, 2 and 3 of a sequence set forth in SEQ ID NO: 5;
(clviii) a V_{H} comprising a sequence set forth in SEQ ID NO: 89 and a V_{L} comprising a sequence set forth in SEQ ID NO: 5;
(clix) a V_{H} comprising CDRs 1, 2 and 3 of a sequence set forth in SEQ ID NO: 90 and a V_{L} comprising CDRs 1, 2 and 3 of a sequence set forth in SEQ ID NO: 5;
(clx) a V_{H} comprising a sequence set forth in SEQ ID NO: 90 and a V_{L} comprising a sequence set forth in SEQ ID NO: 5;
(clxi) a V_{H} comprising CDRs 1, 2 and 3 of a sequence set forth in SEQ ID NO: 91 and a V_{L} comprising CDRs 1, 2 and 3 of a sequence set forth in SEQ ID NO: 5;
(clxii) a V_{H} comprising a sequence set forth in SEQ ID NO: 91 and a V_{L} comprising a sequence set forth in SEQ ID NO: 5;
(clxiii) a V_{H} comprising CDRs 1, 2 and 3 of a sequence set forth in SEQ ID NO: 92 and a V_{L} comprising CDRs 1, 2 and 3 of a sequence set forth in SEQ ID NO: 5;
(clxiv) a V_{H} comprising a sequence set forth in SEQ ID NO: 92 and a V_{L} comprising a sequence set forth in SEQ ID NO: 5;
(clxv) a V_{H} comprising CDRs 1, 2 and 3 of a sequence set forth in SEQ ID NO: 93 and a V_{L} comprising CDRs 1, 2 and 3 of a sequence set forth in SEQ ID NO: 5;
(clxvi) a V_{H} comprising a sequence set forth in SEQ ID NO: 93 and a V_{L} comprising a sequence set forth in SEQ ID NO: 5;
(clxvii) a V_{H} comprising CDRs 1, 2 and 3 of a sequence set forth in SEQ ID NO: 94 and a V_{L} comprising CDRs 1, 2 and 3 of a sequence set forth in SEQ ID NO: 5;
(clxviii) a V_{H} comprising a sequence set forth in SEQ ID NO: 94 and a V_{L} comprising a sequence set forth in SEQ ID NO: 5;
(clxix) a V_{H} comprising CDRs 1, 2 and 3 of a sequence set forth in SEQ ID NO: 95 and a V_{L} comprising CDRs 1, 2 and 3 of a sequence set forth in SEQ ID NO: 5;
(clxx) a V_{H} comprising a sequence set forth in SEQ ID NO: 95 and a V_{L} comprising a sequence set forth in SEQ ID NO: 5;
(clxxi) a V_{H} comprising CDRs 1, 2 and 3 of a sequence set forth in SEQ ID NO: 96 and a V_{L} comprising CDRs 1, 2 and 3 of a sequence set forth in SEQ ID NO: 5;
(clxxii) a V_{H} comprising a sequence set forth in SEQ ID NO: 96 and a V_{L} comprising a sequence set forth in SEQ ID NO: 5;
(clxxiii) a V_{H} comprising CDRs 1, 2 and 3 of a sequence set forth in SEQ ID NO: 97 and a V_{L} comprising CDRs 1, 2 and 3 of a sequence set forth in SEQ ID NO: 5;
(clxxiv) a V_{H} comprising a sequence set forth in SEQ ID NO: 97 and a V_{L} comprising a sequence set forth in SEQ ID NO: 5;
(clxxv) a V_{H} comprising CDRs 1, 2 and 3 of a sequence set forth in SEQ ID NO: 98 and a V_{L} comprising CDRs 1, 2 and 3 of a sequence set forth in SEQ ID NO: 5;
(clxxvi) a V_{H} comprising a sequence set forth in SEQ ID NO: 98 and a V_{L} comprising a sequence set forth in SEQ ID NO: 5;
(clxxvii) a V_{H} comprising CDRs 1, 2 and 3 of a sequence set forth in SEQ ID NO: 99 and a V_{L} comprising CDRs 1, 2 and 3 of a sequence set forth in SEQ ID NO: 5;
(clxxviii) a V_{H} comprising a sequence set forth in SEQ ID NO: 99 and a V_{L} comprising a sequence set forth in SEQ ID NO: 5;
(clxxix) a V_{H} comprising CDRs 1, 2 and 3 of a sequence set forth in SEQ ID NO: 100 and a V_{L} comprising CDRs 1, 2 and 3 of a sequence set forth in SEQ ID NO: 5;
(clxxx) a V_{H} comprising a sequence set forth in SEQ ID NO: 100 and a V_{L} comprising a sequence set forth in SEQ ID NO: 5;
(clxxxi) a V_{H} comprising CDRs 1, 2 and 3 of a sequence set forth in SEQ ID NO: 101 and a V_{L} comprising CDRs 1, 2 and 3 of a sequence set forth in SEQ ID NO: 5;
(clxxxii) a V_{H} comprising a sequence set forth in SEQ ID NO: 101 and a V_{L} comprising a sequence set forth in SEQ ID NO: 5;
(clxxxiii) a V_{H} comprising CDRs 1, 2 and 3 of a sequence set forth in SEQ ID NO: 102 and a V_{L} comprising CDRs 1, 2 and 3 of a sequence set forth in SEQ ID NO: 5;
(clxxxiv) a V_{H} comprising a sequence set forth in SEQ ID NO: 102 and a V_{L} comprising a sequence set forth in SEQ ID NO: 5;
(clxxxv) a V_{H} comprising CDRs 1, 2 and 3 of a sequence set forth in SEQ ID NO: 103 and a V_{L} comprising CDRs 1, 2 and 3 of a sequence set forth in SEQ ID NO: 5;
(clxxxvi) a V_{H} comprising a sequence set forth in SEQ ID NO: 103 and a V_{L} comprising a sequence set forth in SEQ ID NO: 5;
(clxxxvii) a V_{H} comprising CDRs 1, 2 and 3 of a sequence set forth in SEQ ID NO: 104 and a V_{L} comprising CDRs 1, 2 and 3 of a sequence set forth in SEQ ID NO: 5;
(clxxxviii) a V_{H} comprising a sequence set forth in SEQ ID NO: 104 and a V_{L} comprising a sequence set forth in SEQ ID NO: 5;
(clxxxix) a V_{H} comprising CDRs 1, 2 and 3 of a sequence set forth in SEQ ID NO: 105 and a V_{L} comprising CDRs 1, 2 and 3 of a sequence set forth in SEQ ID NO: 5;
(cxc) a V_{H} comprising a sequence set forth in SEQ ID NO: 105 and a V_{L} comprising a sequence set forth in SEQ ID NO: 5;
(cxci) a V_{H} comprising CDRs 1, 2 and 3 of a sequence set forth in SEQ ID NO: 106 and a V_{L} comprising CDRs 1, 2 and 3 of a sequence set forth in SEQ ID NO: 5;
(cxcii) a V_{H} comprising a sequence set forth in SEQ ID NO: 106 and a V_{L} comprising a sequence set forth in SEQ ID NO: 5;
(cxciii) a V_{H} comprising CDRs 1, 2 and 3 of a sequence set forth in SEQ ID NO: 107 and a V_{L} comprising CDRs 1, 2 and 3 of a sequence set forth in SEQ ID NO: 5;
(cxciv) a V_{H} comprising a sequence set forth in SEQ ID NO: 107 and a V_{L} comprising a sequence set forth in SEQ ID NO: 5;
(cxcv) a V_{H} comprising CDRs 1, 2 and 3 of a sequence set forth in SEQ ID NO: 108 and a V_{L} comprising CDRs 1, 2 and 3 of a sequence set forth in SEQ ID NO: 5;
(cxcvi) a V_{H} comprising a sequence set forth in SEQ ID NO: 108 and a V_{L} comprising a sequence set forth in SEQ ID NO: 5;
(cxcvii) a V_{H} comprising CDRs 1, 2 and 3 of a sequence set forth in SEQ ID NO: 109 and a V_{L} comprising CDRs 1, 2 and 3 of a sequence set forth in SEQ ID NO: 5;
(cxcviii) a V_{H} comprising a sequence set forth in SEQ ID NO: 109 and a V_{L} comprising a sequence set forth in SEQ ID NO: 5;
(cxcix) a V_{H} comprising CDRs 1, 2 and 3 of a sequence set forth in SEQ ID NO: 110 and a V_{L} comprising CDRs 1, 2 and 3 of a sequence set forth in SEQ ID NO: 5;
(cc) a V_{H} comprising a sequence set forth in SEQ ID NO: 110 and a V_{L} comprising a sequence set forth in SEQ ID NO: 5;
(cci) a V_{H} comprising CDRs 1, 2 and 3 of a sequence set forth in SEQ ID NO: 111 and a V_{L} comprising CDRs 1, 2 and 3 of a sequence set forth in SEQ ID NO: 5;
(ccii) a V_{H} comprising a sequence set forth in SEQ ID NO: 111 and a V_{L} comprising a sequence set forth in SEQ ID NO: 5;
(cciii) a V_{H} comprising CDRs 1, 2 and 3 of a sequence set forth in SEQ ID NO: 112 and a V_{L} comprising CDRs 1, 2 and 3 of a sequence set forth in SEQ ID NO: 5;
(cciv) a V_{H} comprising a sequence set forth in SEQ ID NO: 112 and a V_{L} comprising a sequence set forth in SEQ ID NO: 5;
(ccv) a V_{H} comprising CDRs 1, 2 and 3 of a sequence set forth in SEQ ID NO: 113 and a V_{L} comprising CDRs 1, 2 and 3 of a sequence set forth in SEQ ID NO: 5;
(ccvi) a V_{H} comprising a sequence set forth in SEQ ID NO: 113 and a V_{L} comprising a sequence set forth in SEQ ID NO: 5;
(ccvii) a V_{H} comprising CDRs 1, 2 and 3 of a sequence set forth in SEQ ID NO: 114 and a V_{L} comprising CDRs 1, 2 and 3 of a sequence set forth in SEQ ID NO: 5;
(ccviii) a V_{H} comprising a sequence set forth in SEQ ID NO: 114 and a V_{L} comprising a sequence set forth in SEQ ID NO: 5;
(ccix) a V_{H} comprising CDRs 1, 2 and 3 of a sequence set forth in SEQ ID NO: 115 and a V_{L} comprising CDRs 1, 2 and 3 of a sequence set forth in SEQ ID NO: 5;
(ccx) a V_{H} comprising a sequence set forth in SEQ ID NO: 115 and a V_{L} comprising a sequence set forth in SEQ ID NO: 5;
(ccxi) a V_{H} comprising CDRs 1, 2 and 3 of a sequence set forth in SEQ ID NO: 116 and a V_{L} comprising CDRs 1, 2 and 3 of a sequence set forth in SEQ ID NO: 5; or
(ccxii) a V_{H} comprising a sequence set forth in SEQ ID NO: 116 and a V_{L} comprising a sequence set forth in SEQ ID NO: 5.

20. The method of any one of claims 1 to 19, wherein the compound is an antibody comprising:
(i) a V_{H} comprising CDRs 1, 2 and 3 of a sequence set forth in SEQ ID NO: 64 and a V_{L} comprising CDRs 1, 2 and 3 of a sequence set forth in SEQ ID NO: 5; or
(ii) a V_{H} comprising a sequence set forth in SEQ ID NO: 64 and a V_{L} comprising a sequence set forth in SEQ ID NO: 5.

21. The method of any one of claims 1 to 20, wherein the method further comprises determining a peripheral blood eosinophil count and/or a peripheral-blood differential eosinophil count in the subject and/or proportion of eosinophils in the subject's sputum.

22. The method of claim 21, wherein the subject has an absolute eosinophil count of at least 200 cells per microliter of blood and/or the proportion of eosinophils in the subject's sputum is at least 1.25%.
